(19) United States
(12) Patent Application Publication
(10) Pub. No.: US 2025/0334591 A1
(43) Pub. Date: Oct. 30, 2025

(54) IDENTIFICATION OF CERVICAL BIOMARKERS

(71) Applicant: Akna Health Inc., Grand Rapids, MI (US)

(72) Inventor: Sascha DREWLO, Grand Rapids, MI (US)

(21) Appl. No.: 19/169,837

(22) Filed: Apr. 3, 2025

Related U.S. Application Data
(63) Continuation of application No. PCT/US23/81437, filed on Nov. 28, 2023.
(60) Provisional application No. 63/385,257, filed on Nov. 29, 2022.
(60) Provisional application No. 63/434,032, filed on Dec. 20, 2022.
(60) Provisional application No. 63/527,779, filed on Jul. 19, 2023.

(51) Int. Cl. G01N 33/68; G01N 33/50

(52) U.S. Cl. CPC G01N 33/689; G01N 33/5091; G01N 2560/00

(57) ABSTRACT

Provided herein are methods and kits for identifying a pregnancy-associated risk or condition in a subject. In embodiments, the methods include detecting levels of at least one biomarker in a substantially cell-free cervical fluid sample obtained from the subject. In embodiments, the methods include detecting levels of biomarkers in a cervical fluid sample including extravillous trophoblasts (EVT) obtained from the subject.

### CROSS REFERENCE TO RELATED APPLICATIONS

This application claims priority to U.S. Provisional Application No. 63/385,257 filed Nov. 29, 2022, U.S. Provisional Application No. 63/434,032 filed Dec. 20, 2022, and U.S. Provisional Application No. 63/527,779 filed Jul. 19, 2023, which are hereby incorporated by reference in their entirety and for all purposes.

### STATEMENT AS TO RIGHTS TO INVENTIONS MADE UNDER FEDERALLY SPONSORED RESEARCH AND DEVELOPMENT

This invention was made with government support under Grant No. 1-R43-EB033715-01 awarded by the National Institute of Health (NIH). The government has certain rights in the invention.

### BACKGROUND

There is a lack of clinical information available on early human placentation, for example from the beginning of pregnancy through the 24^{th} week of the gestational period, a duration of pregnancy when a variety of pathologies originate. Current methods for assessing pregnancy risk often involve the analysis of maternal blood samples. Analysis of blood-based samples is not ideal for early gestation and can be influenced by various extraneous factors. The factors include blood volume, gestational age, and placental size dependent secretion effects which can result in inaccurate detection of biomarkers, leading to failure to diagnose or misdiagnosis of pregnancy-associated risks or conditions. Thus, there is a need for alternative pregnancy risk assessments that are more accurate, reliable, and minimally invasive.

### BRIEF SUMMARY

Provided herein, inter alia, are methods and kits for detecting an altered (e.g. elevated or decreased) level of at least one biomarker relative to a standard control, wherein the altered level of the biomarker is indicative of a pregnancy-associated risk or condition. In embodiments, the one or more biomarker is detectable in a substantially cell free cervical fluid sample obtained from a pregnant subject. Thus, in an aspect is provided a method of identifying a pregnancy-associated risk or condition in a subject, the method including: a) obtaining a cervical fluid sample from a subject; and b) detecting an elevated level or a decreased level of at least one biomarker in the cervical fluid sample relative to a standard control, thereby identifying the pregnancy associated risk or condition, wherein the cervical fluid sample includes no more than 0 to 2 cells per 2 ml volume. In embodiments, the cervical fluid sample includes no more than 1 cell per 1 mL volume.

Provided herein, inter alia, are compositions and methods for detecting cervical biomarkers. In embodiments, the methods include identifying elevated or decreased levels of biomarkers expressed by extravillous trophoblast (EVT) cells relative to a standard control, wherein the elevated or decreased level of the biomarker is indicative of a pregnancy-associated risk or condition. Thus, in an aspect is provided a method for identifying one or more pregnancy-associated risks or conditions in a subject, the method including: a) obtaining a biological sample from the cervix of the subject, the biological sample including extravillous trophoblast (EVT) cells and biological materials derived from the cervix of the subject, wherein the biological materials derived from the cervix of the subject include at least 90% weight by volume (w/v) of the biological sample; b) performing single-cell time-of-flight mass spectrometry (CyTOF-MS) on the biological sample to generate an output; and c) determining the presence or absence of at least one biomarker in the biological sample based on the output, wherein the presence or absence of the at least one biomarker is indicative of an early gestational complication.

In another aspect, a method of identifying one or more pregnancy-associated risks or conditions of a subject is provided, the method including: a) obtaining a biological sample from the cervix of the subject, wherein the biological sample includes extravillous trophoblast (EVT) cells; b) performing single-cell time-of-flight mass spectrometry (CyTOF-MS) on the biological sample to generate an output; and c) determining the presence or absence of at least one biomarker in the biological sample based on the output.

In embodiments, the biological sample further includes biological materials derived from the cervix of the subject. In embodiments, the biological materials derived from the cervix are naturally present in the cervix during pregnancy. In embodiments, the biological materials derived from the cervix comprise mucous, maternal cells, a biological fluid, or any combination thereof. In embodiments, the biological materials derived from the cervix comprise at least 90% of the biological sample.

In embodiments, the methods further comprise washing the biological sample to achieve a single-cell solution. In embodiments, the methods further comprise filtering the biological samples to achieve a single-cell solution.

In embodiments, the at least one biomarker includes a biomarker expressed by an EVT cell. In embodiments, the at least one biomarker is a placental protein. In embodiments, the at least one biomarker includes a biomarker indicative of cervical health. In embodiments, the biomarker is indicative of cervical infection; bleeding; inflammation, and cervical cancer. In embodiments, the at least one biomarker includes a biomarker indicative of a gynecological cancer. In embodiments, the elevated or decreased level of at least one biomarker is indicative of an early gestational complication. In embodiments, the presence or absence of the at least one biomarker is indicative of an early gestational complication.

In embodiments, the early gestational complication is placental dysfunction or insufficiency. In embodiments, the early gestational complication is a risk of early pregnancy loss. In embodiments, the early gestational complication is a risk of preeclampsia. In embodiments, the early gestational complication is a risk of preterm birth. In embodiments, the early gestational complication is a risk of gestational diabetes.

In embodiments, the subject is a pregnant subject. In embodiments, the sample is taken from a pregnant subject that is at least four weeks pregnant. In embodiments, the sample is taken from a pregnant subject that is at least five weeks pregnant.

In embodiments, the EVT purity of the biological sample (e.g. cervical fluid sample) is less than 0.1% (w/v). In embodiments, the biological sample (e.g. cervical fluid sample) includes at least about 25 cells per 1 mL volume. In embodiments, the biological sample (e.g. cervical fluid sample) includes no more than about 25 cells per 1 mL volume. In embodiments, the method is completed in less than 24 hours. In embodiments, the EVT cells comprise extravillous cells residing in or passing through the cervix. In embodiments, the methods further comprise performing flow cytometry analysis on the biological sample.

In embodiments, a kit is used to obtain the biological sample, wherein the kit comprises a) a biohazard spill-proof bag, b) a scraper, c) a cyto-brush, and d) a container including a stabilizing solution.

In another aspect, a non-transitory computer readable medium including machine executable code that, upon execution by one or more computer processors, implements any of the methods above or elsewhere herein.

In another aspect is provided a system including one or more computer processors and computer memory coupled thereto. The computer memory comprises machine executable code that, upon execution by the one or more computer processors, implements any of the methods above or elsewhere herein.

In an aspect a kit for obtaining a cervical fluid sample from a subject is provided, including: a) a collection device; b) a collection container including a stabilizing solution; and a cell lysis solution and/or a cell removal device.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1 shows a diagram of an exemplary workflow of methods for detecting pregnancy-associated biomarkers.

FIG. 2 shows a computer system that is programmed or otherwise configured to implement methods provided herein.

FIG. 3 depicts an exemplary sample collection schedule. The figure illustrates a sample collection schedule including collection of biological sample (e.g. cervical fluid sample) in the early gestational period of pregnancy (e.g. prior to 24 weeks). The collection schedule for obtaining cervical fluid sample is compared to the collection schedule for obtaining blood sample.

FIG. 4 shows identification of 200-25 HLA-G+ trophoblast Jeg3 cells (left peak) spiked into a pool of 500,000 PBMC cells (right peak) using CyTOF.

FIG. 5 depicts a table of a protein marker panel to identify and characterize cEVT and other cell types in a cervical fluid sample and a subset in the maternal blood at matching time points.

FIGS. 6A-6B show identification of the EVT-like cell populations in cervical specimens by CyTOF. FIG. 6A shows a tSNE plot of HLA-G+cEVT cells (circled).

FIG. 6B shows that median expressions of trophoblast lineage markers are identified by HLA-G+ (left) in the trophoblast cells as compared to other cells in the clinical specimen HLA-G-(right).

FIGS. 7A-7B show CyTOF identification of HLA-G cEVT cells in control and FGR pregnancy. FIG. 7A shows an example for gating of HLA-G (EVT) and DNA (cell marker) in FGR pregnancy to identify cEVT cells. FIG. 7B shows cEVT cell frequency.

FIG. 8 shows cEVT cell protein expression profiles of aberrant (intrauterine growth restriction IUGR) vs. healthy (control) pregnancies using CyTOF. p<0.05. Graphs show 25th to 75th percentiles, with medians (horizontal lines in the blocks). The whiskers indicate 1.5×Inter Quartile Range (3rd quartile−1st quartile).

FIG. 9 shows levels of PLGF as measured in cervical fluid samples obtained from eight pregnant patients.

FIG. 10 shows levels of alpha-fetoprotein (AFP), a biomarker associated with abnormal placentation, as detected in cervical fluid samples obtained from eight patients. The cell-free detection method shows that one patient had elevated levels of AFP.

FIG. 11 shows a graph displaying sFlt-1/PLGF (PGF) ratio as detected in cervical fluid samples obtained from eight patients during the first trimester. The ratio is predictive of risk or presence of preeclampsia in pregnant patients.

FIG. 12 shows levels of PAPPA as measured in cervical fluid samples obtained from eight patients. PAPA was found in all 8 pregnant patient samples tested. Patient 6's data showed a marked decrease in PAPP-A levels compared to healthy controls.

FIG. 13 illustrates detection of soluble FMS-like tyrosine kinase-1 (sFlt-1) in cervical fluid samples in pregnant patients.

FIGS. 14A-14B illustrate that cell free placental/fetal DNA is present in cervical fluid samples. Using PCR, a sequence of the male-specific SRY gene was detected, thereby identifying three male fetuses (FIG. 14A). The control XIST gene was further identified by PCR, thereby validating successful PCR detection of the target gene (FIG. 14B).

### DETAILED DESCRIPTION

The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Examples of such techniques are available in the literature.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when. The cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

For specific proteins described herein, the named protein includes any of the protein's naturally occurring forms, variants or homologs that maintain the protein activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. In embodiments, the term gene includes a fragment or a portion of a gene. For example, the term gene may refer to a portion or fragment of a gene encoding a protein provided herein. Further, a “protein gene product” is a protein expressed from a particular gene.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

A “detectable agent” or “detectable moiety” is a composition, substance, element, or compound; or moiety thereof; detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fc, ⁵⁹Fc, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Rc, ¹⁸⁸Rc, ¹⁸⁹Rc, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Cc, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

“Contacting” is used in accordance with its plain ordinary meaning and refers to the process of allowing at least two distinct species (e.g. antibodies and antigens, biomarker and detection agent) to become sufficiently proximal to react, interact, or physically touch. It should be appreciated; however, that the resulting reaction product can be produced directly from a reaction between the added reagents or from an intermediate from one or more of the added reagents which can be produced in the reaction mixture. In embodiments, contacting refers to allowing an antibody (e.g. detectable-moiety conjugated antibody) contact an EVT cell. In embodiments, contacting refers to allowing an antibody (e.g. detectable-moiety conjugated antibody) contact a biomarker.

The term “contacting” may include allowing two species to react, interact, or physically touch, wherein the two species may be, for example, a cervical fluid sample and a reagent (e.g. a cell lysis solution, a nucleic acid stabilizing solution, an antibody, etc.) from a kit as provided herein.

A “cell” as used herein, may refer to a living cell, a dead cell, or a cell fragment. When the term “cell” refers to a living cell, the cell carries out metabolic or other function sufficient to preserve or replicate its genomic DNA. In embodiments, a living cell can be identified by well-known methods in the art including, for example, presence of an intact membrane, staining by a particular dye, ability to produce progeny or, in the case of a gamete, ability to combine with a second gamete to produce a viable offspring. When the term “cell” refers to a dead cell, the cell may be identified by loss of membrane integrity. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “isolated”, when applied to a cell denotes that the cell has been removed from other components with which it is associated with in the natural state. For example, when a cell is isolated from a sample (e.g. a cervical fluid sample, a biological sample), the cell is removed from other components naturally occurring in the sample. In embodiments, isolating the cell does not denote contacting a cell with an antibody. Specifically, when a cell is bound to an antibody, the cell is not considered to be isolated if the cell-antibody complex is not removed from other components naturally occurring in the sample. In embodiments, contacting a cell with a detectable moiety does not denote isolating the cell. For example, in embodiments, isolating an EVT cell does not denote contacting an EVT cell with a detectable moiety conjugated antibody. When applied to a nucleic acid or protein, isolated denotes that the nucleic acid or protein is essentially free of other cellular components with which it is associated in the natural state. It can be, for example, in a homogeneous state and may be in either a dry or aqueous solution. Purity and homogenicity are typically determined using analytical chemistry techniques such as polyacrylamide gel electrophoresis or high performance liquid chromatography. A protein that is the predominant species present in a preparation is substantially purified.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. In embodiments, a biological sample includes sections of tissues such as biopsy and autopsy samples, and frozen sections taken for histological purposes. In embodiments, the tissue is obtained from the cervix of the subject. In embodiments, a biological sample is cell-free or substantially cell free. For example, in embodiments, a biological sample includes a cervical fluid sample that is substantially cell free. In embodiments, the biological sample is a cervical fluid sample. In embodiments, a biological sample includes bodily fluids such as blood and blood fractions or products (e.g., serum, plasma, platelets, red blood cells, and the like), sputum, or tissue. In embodiments, the biological tissue includes cultured cells (e.g., primary cultures, explants, and transformed cells) derived from cells obtained from a subject. For example, cells obtained from the cervix of a sample may be cultured in media thereby producing cultured cells. In embodiments, a biological sample includes material obtained from or derived from the uterus, cervix, or vagina of a subject. A biological sample may generally include organic compounds, tissue, cellular components, body compatible fluids, biomass, bio-composites, biocompatible materials, antibodies, DNA, RNA, proteins, molecules for therapeutic purposes, and other organic substances or substances native to a living organism. Typically, a biological sample is obtained from a eukaryotic organism, such as a mammal such as a primate e.g., chimpanzee or human; cow; dog; cat; a rodent, e.g., guinea pig, rat, mouse; rabbit; or a bird; reptile; or fish.

The compositions of a biological sample can depend on the origin of the sample. Biological samples naturally present in the cervical canal (e.g. cervical fluid sample) can include, but are not limited to, DNA, RNA, peptides, proteins, polypeptides, mucous, blood, and cells (e.g., EVT trophoblast cells, epithelial cells, glandular cells). Biological samples naturally present in the cervical canal of a pregnant subject can include maternal-derived biological materials and/or fetal-derived biological materials. In embodiments, a biological sample may be cell free or substantially cell free.

“Cervical fluid sample” refers to bodily fluid obtained from the cervix of an organism. The cervical fluid sample may be obtained from any portion of the cervix. For example, the cervical fluid sample may be obtained from the endo cervix, the endocervical canal, or the exo cervix. In embodiments, the cervical fluid sample may be obtained from the internal OS or the external OS. In embodiments, the cervical fluid sample may be obtained as a cervical fluid secretion or a cervical fluid emission that is collected outside of the cervix (e.g. in a menstrual cup or a collection disc). In embodiments, the cervical fluid sample includes bodily fluids that do not originate from the cervix and have accumulated in the cervix. For example, in embodiments, the cervical fluid sample may include other body fluids or biological materials such as mucous, blood, or fetal DNA. In embodiments, cervical fluid sample can include DNA, RNA, peptides, proteins, polypeptides, mucous, blood, and cells (e.g., EVT cells, epithelial cells, glandular cells). Levels of biomakers in the cervical fluid sample may be indicative of a particular condition or disease (e.g. pregnancy-associated risk).

The terms “biomarker” and “marker” as used interchangeably herein, generally refer to a biomolecule or fragment of a biomolecule, the change and/or detection of which may be associated with a particular physical condition or state. These biomarkers can include any suitable analyte, but are not limited to biomolecules, including nucleotides, nucleic acids, nucleosides, amino acids, sugars, fatty acids, steroids, metabolites, peptides, polypeptides, proteins, carbohydrates, fats, hormones, antibodies, regions of interest that serve as surrogates for biological macromolecules, and combinations thereof (e.g., glycoproteins, ribonucleoproteins, lipoproteins). In embodiments, the biomarker is a cell fragment, microvesicle, ectosome, microparticle, extracellular vesicle, or micelle. The terms also include portions or fragments of biomolecules.

The presence of a biomarker can refer to a biomarker being present, as opposed to a control sample where the biomarker is absent. Alternatively, the presence of a biomarker can refer to a biomarker being upregulated when compared to a control. Similarly, the absence of a biomarker can refer to a biomarker being absent, as opposed to the control sample where the biomarker is present. Alternatively, the absence of a biomarker can refer to a biomarker being downregulated when compared to a control.

A “control” or “standard control” refers to a sample, measurement, or value that serves as a reference, usually a known reference, for comparison to a test sample, measurement, or value. For example, a test sample can be taken from a patient suspected of having a given disease or condition (e.g. pregnancy-induced hypertension, placental insufficiency, etc.) and compared to a known normal (e.g. subject without the disease or condition, a non-pregnant subject) individual (e.g. a standard control subject). A standard control can also represent an average measurement or value gathered from a population of similar individuals (e.g. standard control subjects) that do not have a given disease or condition (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient”, “subject” or “subject in need thereof” refers to a living organism that can be diagnosed by a method, or kit or composition as provided herein. In embodiments, the subject is suffering from or prone to a disease or condition (e.g. pregnancy, pregnancy-induced hypertension, placental insufficiency, etc.). Non-limiting examples include humans, other mammals, bovines, rats, mice, dogs, monkeys, goat, sheep, cows, deer, and other non-mammalian animals. In some embodiments, a patient is human. In embodiments, the subject is pregnant. In embodiments, the subject is at least 4 weeks pregnant, as measured by gestational age. In embodiments, the subject is at least 5 weeks pregnant, as measured by gestational age. In embodiments, the subject is at least 6 weeks pregnant, as measured by gestational age. In embodiments, the subject is at least 7 weeks pregnant, as measured by gestational age. In embodiments, the subject is at least 8 weeks pregnant, as measured by gestational age. In embodiments, the subject is at least 9 weeks pregnant, as measured by gestational age. In embodiments, the subject is at least 10 weeks pregnant, as measured by gestational age.

The terms “disease” or “condition” refer to a state of being or health status of a patient or subject capable of being diagnosed with the methods or kits or compositions provided herein. The disease or condition may be placental insufficiency, pregnancy-induced hypertension, placental abruption, pregnancy loss, miscarriage, preeclampsia, eclampsia, hemolysis, elevated liver enzymes and low platelet count (HELLP) syndrome, fetal growth restriction, intrauterine growth restriction, preterm birth, low birthweight, placenta percreta, placenta increta, placenta previa, gestational hypertension, gestational diabetes, gestational thrombosis, stillbirth, or placental infarction. The disease or condition may be a disease or condition typically associated with pregnancy. In embodiments, the disease or condition is an early gestational complication (e.g. a state, disease or condition that occurs prior to the 24^{th} week of pregnancy as measured by gestational age).

The term “associated” or “associated with” in the context of a substance or substance activity or function associated with a disease or condition (e.g. a protein associated disease, a condition associated with a protein activity) means that the disease or condition (e.g. placental insufficiency, gestational diabetes, pregnancy-induced hypertension, placental abruption, pregnancy loss, miscarriage, preeclampsia etc.) is caused by (in whole or in part), or a symptom of the disease or condition is caused by (in whole or in part) the substance or substance activity or function. As used herein, what is described as being associated with a disease or condition, if a causative agent, could be a target for treatment of the disease or condition. In embodiments, the state or condition of the subject may be associated with the condition of pregnancy. In embodiments, “associated” or “associated with” in the context of a disease or condition means that the disease or conditions associated with the state of a subject. In embodiments, the state of the subject is the state of being pregnant. For example, in embodiments, pre-eclampsia may be referred to as a pregnancy-associated risk or condition. In embodiments, the pregnancy-associated risk or condition is placental insufficiency, pregnancy-induced hypertension, placental abruption, pregnancy loss, miscarriage, preeclampsia, eclampsia, hemolysis, elevated liver enzymes and low platelet count (HELLP) syndrome, fetal growth restriction, intrauterine growth restriction, preterm birth, low birthweight, placenta percreta, placenta increta, placenta previa, gestational hypertension, gestational thrombosis, stillbirth, or placental infarction.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms. In embodiments, aberrant may refer to the level of a biomarker provided herein that is higher or lower than the level of a biomarker in a standard control.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Methods for Identifying Pregnancy-Associated Risks and Conditions

Provided herein, inter alia, are methods for identifying a pregnancy-associated risk or condition in a subject including detecting a level of at least one biomarker in a cervical fluid sample obtained from the subject, wherein the level of the biomarker is indicative of the pregnancy risk or condition. “Marker” or “biomarker” is used in accordance with its plain ordinary meaning and refers to a measurable substance or compound in a biological sample that is indicative of a process or of a condition or a disease. In the case of a pregnant subject a biomarker may be indicative of a process or of a condition or a disease of the embryo or fetus. For example, a biomarker (e.g. a gene or fragment thereof) may be indicative of the sex of a fetus. In embodiments, the biomarker may be released from a cell (e.g. placenta-specific cell) and accumulate in the cervical fluid of a pregnant subject. For example, the biomarker may be a protein expressed by a cell and subsequently released from the cell. In one example, the biomarker may be expressed and released by a cervical cell. In another example, the biomarker may be expressed and secreted by a non-cervical cell, and subsequently accumulate in the cervix of a pregnant subject. In embodiments, the biomarker is expressed by a placenta-specific cell (e.g. an EVT). Thus, in an aspect is provided a method of identifying a pregnancy-associated risk or condition in a subject, the method including: a) obtaining a cervical fluid sample from a subject; and b) detecting an elevated level or a decreased level of at least one biomarker in the cervical fluid sample relative to a standard control, thereby identifying the pregnancy associated risk or condition, wherein the cervical fluid sample includes no greater than about 1 cell per 1 milliliter (mL volume).

The term “cervical fluid sample” refers to a biological sample obtained from the cervix, including from the cervical canal, endocervix and exocervix. In embodiments, the terms “cervical fluid sample” and “biological sample” are interchangeable. The cervical fluid sample may be obtained directly from the cervix, or may be obtained outside of the cervix using a collection device (e.g. via a menstrual cup, etc.). The cervical fluid sample may include components originating from the cervix. The cervical fluid sample may include components derived from other tissue or cells originating outside the cervix (e.g. placenta, amniotic fluid, etc.) which have subsequently accumulated in the cervix. In embodiments, a cervical fluid sample includes material (e.g. mucous, proteins, DNA) which has passed through the cervix. In embodiments, the cervical fluid sample may be collected via a cytobrush, a cytological brush, a catheter, a lavage device, a menstrual cup, or a collection disc. For example, the cervical fluid sample may be obtained from outside of the cervix using a collection device (e.g. cytobrush, a cytological brush, a catheter, a lavage device, a menstrual cup, or a collection disc, etc.) as described herein.

In embodiments, the cervical fluid sample may include components that originated from the uterus, placenta or fetus. In embodiments, the cervical fluid sample may include body fluids or biological materials originating from the fetus, amniotic sac, placenta, or fetus. For example, the cervical fluid sample may include mucous, blood, protein, maternal DNA, or fetal DNA. In embodiments, the cervical fluid sample includes mucous. In embodiments, the cervical fluid sample includes blood. In embodiments, the cervical fluid sample includes protein. In embodiments, the cervical fluid sample includes maternal DNA. In embodiments, the cervical fluid sample includes fetal DNA. In embodiments, the cervical fluid sample includes biological material derived from the subject (e.g., maternal cells). In embodiments, the cervical fluid sample includes biological material derived from the fetus/placenta (e.g., fetal cells or placenta-specific cell). In embodiments, the biological materials are derived from the cervix of the subject. In embodiments, the biological materials derived from the cervix are naturally present in the cervix during pregnancy. For example, the biological materials be derived from cervical tissue or cervical cells during pregnancy. Alternatively, in embodiments, the biological materials may be derived from the uterus, placenta, or amniotic fluid and have accumulated in the cervix during pregnancy. In embodiments, the biological materials derived from the cervix comprise mucous, maternal cells, a biological fluid, or any combination thereof.

Thus, the cervical fluid sample may include, in embodiments, biological material that has passed through the cervix. For example, in embodiments, the cervical fluid may include a discharge that has passed through the cervix and is obtained outside the cervix. Thus, in embodiments, the cervical fluid sample may be derived from a cervical secretion, a cervical mucous, a cervical emission, a cervical excretion, or a cervical discharge obtained from the exo-cervix, vagina, or vaginal opening. In embodiments, the cervical fluid sample is derived from a cervical secretion, a cervical mucous, a cervical emission, a cervical excretion, or a cervical discharge. In embodiments, the cervical fluid sample is derived from a cervical secretion. In embodiments, the cervical fluid sample is derived from a cervical mucous. In embodiments, the cervical fluid sample is derived from a cervical emission. In embodiments, the cervical fluid sample is derived from a cervical excretion. In embodiments, the cervical fluid sample is derived from a cervical discharge.

In embodiments, the cervical fluid sample is obtained from the subject during the early gestational period. As used herein, “early gestational age” or “early gestational period” and the like refer to the duration of pregnancy spanning from the first day of the pregnant subject's last menstrual cycle to the 24^{th} week of pregnancy. In embodiments, the cervical fluid sample is obtained from the subject prior to a gestational age of 24 weeks, wherein the gestational age is measured from the number of weeks that have elapsed since the first day of the subject's last menstrual period. For example, the 6^{th} week of pregnancy in terms of gestational age means that 6 weeks have passed since the first day of the last menstrual period of the pregnant subject. In embodiments, the cervical fluid sample is obtained from the subject between 0 weeks and 24 weeks of pregnancy. In embodiments, the cervical fluid sample is obtained from the subject between 1 week and 24 weeks of pregnancy. In embodiments, the cervical fluid sample is obtained from the subject between 2 weeks and 24 weeks of pregnancy. In embodiments, the cervical fluid sample is obtained from the subject between 3 weeks and 24 weeks of pregnancy. In embodiments, the cervical fluid sample is obtained from the subject between 4 weeks and 24 weeks of pregnancy. In embodiments, the cervical fluid sample is obtained from the subject between 5 weeks and 24 weeks of pregnancy. In embodiments, the cervical fluid sample is obtained from the subject between 6 weeks and 24 weeks of pregnancy. In embodiments, the cervical fluid sample is obtained from the subject between 7 weeks and 24 weeks of pregnancy. In embodiments, the cervical fluid sample is obtained from the subject between 8 weeks and 24 weeks of pregnancy. In embodiments, the cervical fluid sample is obtained from the subject between 9 weeks and 24 weeks of pregnancy. In embodiments, the cervical fluid sample is obtained from the subject between 10 weeks and 24 weeks of pregnancy. In embodiments, the cervical fluid sample is obtained from the subject between 11 weeks and 24 weeks of pregnancy. In embodiments, the cervical fluid sample is obtained from the subject between 12 weeks and 24 weeks of pregnancy. In embodiments, the cervical fluid sample is obtained from the subject between 13 weeks and 24 weeks of pregnancy. In embodiments, the cervical fluid sample is obtained from the subject between 14 weeks and 24 weeks of pregnancy. In embodiments, the cervical fluid sample is obtained from the subject between 15 weeks and 24 weeks of pregnancy. In embodiments, the cervical fluid sample is obtained from the subject between 16 weeks and 24 weeks of pregnancy. In embodiments, the cervical fluid sample is obtained from the subject between 17 weeks and 24 weeks of pregnancy. In embodiments, the cervical fluid sample is obtained from the subject between 18 weeks and 24 weeks of pregnancy. In embodiments, the cervical fluid sample is obtained from the subject between 19 weeks and 24 weeks of pregnancy. In embodiments, the cervical fluid sample is obtained from the subject between 20 weeks and 24 weeks of pregnancy. In embodiments, the cervical fluid sample is obtained from the subject between 21 weeks and 24 weeks of pregnancy. In embodiments, the cervical fluid sample is obtained from the subject between 22 weeks and 24 weeks of pregnancy. In embodiments, the cervical fluid sample is obtained from the subject between 23 weeks and 24 weeks of pregnancy.

In embodiments, the cervical fluid sample is obtained from the subject between 0 weeks and 23 weeks of pregnancy. In embodiments, the cervical fluid sample is obtained from the subject between 0 weeks and 22 weeks of pregnancy. In embodiments, the cervical fluid sample is obtained from the subject between 0 weeks and 21 weeks of pregnancy. In embodiments, the cervical fluid sample is obtained from the subject between 0 weeks and 20 weeks of pregnancy. In embodiments, the cervical fluid sample is obtained from the subject between 0 weeks and 19 weeks of pregnancy. In embodiments, the cervical fluid sample is obtained from the subject between 0 weeks and 18 weeks of pregnancy. In embodiments, the cervical fluid sample is obtained from the subject between 0 weeks and 17 weeks of pregnancy. In embodiments, the cervical fluid sample is obtained from the subject between 0 weeks and 16 weeks of pregnancy. In embodiments, the cervical fluid sample is obtained from the subject between 0 weeks and 15 weeks of pregnancy. In embodiments, the cervical fluid sample is obtained from the subject between 0 weeks and 14 weeks of pregnancy. In embodiments, the cervical fluid sample is obtained from the subject between 0 weeks and 13 weeks of pregnancy. In embodiments, the cervical fluid sample is obtained from the subject between 0 weeks and 12 weeks of pregnancy. In embodiments, the cervical fluid sample is obtained from the subject between 0 weeks and 11 weeks of pregnancy. In embodiments, the cervical fluid sample is obtained from the subject between 0 weeks and 10 weeks of pregnancy. In embodiments, the cervical fluid sample is obtained from the subject between 0 weeks and 9 weeks of pregnancy. In embodiments, the cervical fluid sample is obtained from the subject between 0 weeks and 8 weeks of pregnancy. In embodiments, the cervical fluid sample is obtained from the subject between 0 weeks and 7 weeks of pregnancy. In embodiments, the cervical fluid sample is obtained from the subject between 0 weeks and 6 weeks of pregnancy. In embodiments, the cervical fluid sample is obtained from the subject between 0 weeks and 5 weeks of pregnancy. In embodiments, the cervical fluid sample is obtained from the subject between 0 weeks and 4 weeks of pregnancy. In embodiments, the cervical fluid sample is obtained from the subject between 0 weeks and 3 weeks of pregnancy. In embodiments, the cervical fluid sample is obtained from the subject between 0 weeks and 2 weeks of pregnancy. In embodiments, the cervical fluid sample is obtained from the subject between 0 weeks and 1 weeks of pregnancy. In embodiments, the cervical fluid sample is obtained from the subject at 0 weeks, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of pregnancy.

For the methods provided herein including embodiments thereof, the cervical fluid sample may be obtained from the subject at any time in pregnancy. When measured by conceptional age (e.g. the length of pregnancy from the time of conception), the cervical fluid sample may be obtained from the subject at 1 day pregnant, 2 days pregnant, 3 days pregnant, 4 days pregnant, 5 days pregnant, 6 days pregnant, 1 week pregnant, 2 weeks pregnant, 3 weeks pregnant, 4 weeks pregnant, 5 weeks pregnant, 6 weeks pregnant, 7 weeks pregnant, 8 weeks pregnant, 9 weeks pregnant, 10 weeks pregnant, 11 weeks pregnant, 12 weeks pregnant, 13 weeks pregnant, 14 weeks pregnant, 15 weeks pregnant, 16 weeks pregnant, 17 weeks pregnant, 18 weeks pregnant, 19 weeks pregnant, 20 weeks pregnant, 21 weeks pregnant, 22 weeks pregnant, 23 weeks pregnant, 24 weeks pregnant, 25 weeks pregnant, 26 weeks pregnant, 27 weeks pregnant, 28 weeks pregnant, 29 weeks pregnant, 30 weeks pregnant, 31 weeks pregnant, 32 weeks pregnant, 33 weeks pregnant, 34 weeks pregnant, 35 weeks pregnant, 36 weeks pregnant, 37 weeks pregnant, 38 weeks pregnant, 39 weeks pregnant, 40 weeks pregnant, or more than 40 weeks pregnant. Thus, for the method provided herein, in embodiments, the subject is 1 day pregnant, 2 days pregnant, 3 days pregnant, 4 days pregnant, 5 days pregnant, 6 days pregnant, 1 week pregnant, 2 weeks pregnant, 3 weeks pregnant, 4 weeks pregnant, 5 weeks pregnant, 6 weeks pregnant, 7 weeks pregnant, 8 weeks pregnant, 9 weeks pregnant, 10 weeks pregnant, 11 weeks pregnant, 12 weeks pregnant, 13 weeks pregnant, 14 weeks pregnant, 15 weeks pregnant, 16 weeks pregnant, 17 weeks pregnant, 18 weeks pregnant, 19 weeks pregnant, 20 weeks pregnant, 21 weeks pregnant, 22 weeks pregnant, 23 weeks pregnant, 24 weeks pregnant, 25 weeks pregnant, 26 weeks pregnant, 27 weeks pregnant, 28 weeks pregnant, 29 weeks pregnant, 30 weeks pregnant, 31 weeks pregnant, 32 weeks pregnant, 33 weeks pregnant, 34 weeks pregnant, 35 weeks pregnant, 36 weeks pregnant, 37 weeks pregnant, 38 weeks pregnant, 39 weeks pregnant, 40 weeks pregnant, or more than 40 weeks pregnant when measured by conceptional age (e.g. the length from pregnancy from the time of conception).

In embodiments, the cervical fluid sample is cell free or substantially cell free. In embodiments, the term substantially cell free refers to no more than about 5 cells per 1 mL volume. In embodiments, the term substantially cell free refers to no more than about 4 cells per 1 mL volume. In embodiments, the term substantially cell free refers to no more than about 3 cells per 1 mL volume. In embodiments, the term substantially cell free refers to no more than about 2 cells per 1 mL volume. In embodiments, the term substantially cell free refers to no more than about 1 cell per 1 mL volume. Thus, in an aspect is provided a method of identifying a pregnancy-associated risk or condition in a subject, the method including: a) obtaining a cervical fluid sample from a subject; and b) detecting an elevated level or a decreased level of at least one biomarker in the cervical fluid sample relative to a standard control, thereby identifying the pregnancy associated risk or condition, wherein the cervical fluid sample is substantially cell free. Thus, for the methods provided herein, in embodiments, the cervical fluid sample includes no more than 5 cells per 1 mL volume. In embodiments, the cervical fluid sample includes no more than 4 cells per 1 mL volume. In embodiments, the cervical fluid sample includes no more than 3 cells per 1 mL volume. In embodiments, the cervical fluid sample includes no more than 2 cells per 1 mL volume. In embodiments, the cervical fluid sample includes no more than 1 cells per 1 mL volume. In embodiments, the cervical fluid sample does not include any cells.

In embodiments, substantially cell free refers to a sample including no more than about 20 ng cells per 1 mL volume. Thus, for the method provided herein, in embodiments, the cervical fluid sample includes no more than about 20 ng cells per 1 mL volume, no more than about 18 ng cells per 1 mL volume, no more than about 16 ng cells per 1 mL volume, no more than about 14 ng cells per 1 mL volume, no more than about 12 ng cells per 1 mL volume, no more than about 10 ng cells per 1 mL volume, no more than about 8 ng cells per 1 mL volume, no more than about 6 ng cells per 1 mL volume, no more than about 4 ng cells per 1 mL volume, no more than about 2 ng cells per 1 mL volume, or no more than about 1 ng cells per 1 mL volume. In embodiments, the cervical fluid sample includes no more than about 20 ng cells per 1 mL volume. In embodiments, the cervical fluid sample includes no more than about 18 ng cells per 1 mL volume. In embodiments, the cervical fluid sample includes no more than about 16 ng cells per 1 mL volume. In embodiments, the cervical fluid sample includes no more than about 14 ng cells per 1 mL volume. In embodiments, the cervical fluid sample includes no more than about 12 ng cells per 1 mL volume. In embodiments, the cervical fluid sample includes no more than about 10 ng cells per 1 mL volume. In embodiments, the cervical fluid sample includes no more than about 8 ng cells per 1 mL volume. In embodiments, the cervical fluid sample includes no more than about 6 ng cells per 1 mL volume. In embodiments, the cervical fluid sample includes no more than about 4 ng cells per 1 mL volume. In embodiments, the cervical fluid sample includes no more than about 2 ng cells per 1 mL volume. In embodiments, the cervical fluid sample includes 0 ng cells per 1 mL volume. The cells may be living cells, dead cells, fragments of cells, or cell particles. Therefore, in embodiments, the cervical fluid sample may include partial fragments of cells. In embodiments, the cervical fluid sample does not include dead cells, fragments of cells or cell particles.

In embodiments, the cervical fluid sample includes about 0.025 ng to about 5 ng cells per 1 ml volume. In embodiments, the cervical fluid sample includes about 0.05 ng to about 5 ng of cells. In embodiments, the cervical fluid sample includes about 0.1 ng to about 5 ng of cells per 1 ml volume. In embodiments, the cervical fluid sample includes about 0.15 ng to about 5 ng of cells per 1 ml volume. In embodiments, the cervical fluid sample includes about 0.2 ng to about 5 ng of cells per 1 ml volume. In embodiments, the cervical fluid sample includes about 0.25 ng to about 5 ng of cells per 1 ml volume. In embodiments, the cervical fluid sample includes about 0.3 ng to about 5 ng of cells per 1 ml volume. In embodiments, the cervical fluid sample includes about 0.35 ng to about 5 ng of cells per 1 ml volume. In embodiments, the cervical fluid sample includes about 0.4 ng to about 5 ng of cells per 1 ml volume. In embodiments, the cervical fluid sample includes about 0.45 ng to about 5 ng of cells per 1 ml volume.

In embodiments, the cervical fluid sample includes about 0.025 ng to about 0.45 ng cells per 1 ml volume. In embodiments, the cervical fluid sample includes about 0.025 ng to about 0.4 ng cells per 1 ml volume. In embodiments, the cervical fluid sample includes about 0.025 ng to about 0.35 ng cells per 1 ml volume. In embodiments, the cervical fluid sample includes about 0.025 ng to about 0.3 ng cells per 1 ml volume. In embodiments, the cervical fluid sample includes about 0.025 ng to about 0.25 ng cells per 1 ml volume. In embodiments, the cervical fluid sample includes about 0.025 ng to about 0.2 ng cells per 1 ml volume. In embodiments, the cervical fluid sample includes about 0.025 ng to about 0.15 ng cells per 1 ml volume. In embodiments, the cervical fluid sample includes about 0.025 ng to about 0.1 ng cells per 1 ml volume. In embodiments, the cervical fluid sample includes about 0.025 ng to about 0.05 ng cells per 1 ml volume. In embodiments, the cervical fluid sample includes about 0.025 ng, 0.05 ng, 0.1 ng, 0.15 ng, 0.2 ng, 0.25 ng, 0.3 ng, 0.35 ng, 0.4 ng, 0.45 ng, or 0.5 ng cells per 1 mL volume.

In embodiments, a cervical fluid sample includes cervical cells (e.g. no greater than about 2 cervical cells per 2 mL volume, or no greater than about 1 cervical cell per 2 mL volume). Cervical cells include cells that line the surface of the cervix. In embodiments, cervical cells include glandular cells, which have a column-shaped appearance, and squamous cells, which are thin and flat. In embodiments, the cervical cells comprise cells that have accumulated in the cervix (e.g., EVT cells or other placental cells).

In embodiments, the cervical fluid sample can be collected through a pap smear or other procedure that allows for the collection of cervical material (e.g., a diva cup). In embodiments, obtaining a cervical fluid sample includes use of a cytobrush, a cytological brush, a catheter, a lavage device, a menstrual cup, or a collection disc. In embodiments, the cervical fluid sample can be obtained from a swab that has contacted the subject. In embodiments, the cervical fluid sample can be obtained from the vaginal canal (e.g., using a cytobrush). In embodiments, the cervical fluid sample can be obtained from or derived from a body fluid sample, a tissue biopsy sample, a necropsy sample, or a swab or cytobrush sample. In embodiments, the cervical fluid sample can be obtained from the endo cervix or the exo cervix. The endocervix refers to the opening of the cervix that leads to the uterus. The exocervix (e.g. ectocervix) refers to the outer region of the cervix that leads to the vagina. Thus, in embodiments, obtaining a cervical fluid sample may include use of a cytobrush, a cytological brush, a catheter, a lavage device, a menstrual cup, or a collection disc.

In embodiments, when a biomarker is detectable in the cervical fluid sample, the biomarker is identified as being an elevated level compared to a control. In embodiments, when a biomarker undetectable in the cervical fluid sample, the biomarker is identified as being a decreased level compared to a control.

In embodiments, a control (e.g., standard control) is the level of a biomarker in a cervical fluid sample obtained from a subject who does not have a pregnancy-associated risk or condition (e.g. eclampsia, preeclampsia, risk of miscarriage, risk of pre-term birth, etc.). In embodiments, a control (e.g., standard control) is the average level of a biomarker in cervical fluid samples taken from a group of subjects who do not have a pregnancy-associated risk or condition. In embodiments, the control is the level of a biomarker in a cervical fluid sample obtained from a pregnant subject or the average level of a biomarker in the cervical fluid samples obtained from a group of pregnant subjects. In embodiments, the control is the level of a biomarker in a cervical fluid sample obtained from a non-pregnant subject or the average level of a biomarker in the cervical fluid samples obtained from a group of non-pregnant subjects.

For the methods provided herein, in embodiments, the elevated level of a biomarker is a level that is at least about 5%, at least about 10%, at least about 15%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 1,000%, at least about 5,000%, or at least about 10,000% greater than a control (e.g. the level of the biomarker from a cervical fluid sample of a subject who does not have a pregnancy-associated risk or condition, the level of the biomarker from a cervical fluid sample of a subject who is not pregnant).

In embodiments, the elevated level of a biomarker is a level that is increased at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, or at most about 5% of a control (e.g. the level of the biomarker from a cervical fluid sample of a subject who does not have a pregnancy-associated risk or condition, the level of the biomarker from a cervical fluid sample of a subject who is not pregnant).

In embodiments, the elevated level of a biomarker is increased by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold of a control (e.g. the level of the biomarker from a cervical fluid sample of a subject who does not have a pregnancy-associated risk or condition, the level of the biomarker from a cervical fluid sample of a subject who is not pregnant).

In embodiments, the level of an elevated biomarker is increased by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold of a control (e.g. the level of the biomarker from a cervical fluid sample of a subject without the pregnancy-associated risk or condition, the level of the biomarker from a cervical fluid sample of a subject who is not pregnant).

In embodiments, the level of the biomarker is elevated by at least 5% relative to the control. In embodiments, the level of the biomarker is elevated by at least 10% relative to the control. In embodiments, the level of the biomarker is elevated by at least 15% relative to the control. In embodiments, the level of the biomarker is elevated by at least 20% relative to the control. In embodiments, the level of the biomarker is elevated by at least 25% relative to the control. In embodiments, the level of the biomarker is elevated by at least 30% relative to the control. In embodiments, the level of the biomarker is elevated by at least 35% relative to the control. In embodiments, the level of the biomarker is elevated by at least 40% relative to the control. In embodiments, the level of the biomarker is elevated by at least 45% relative to the control. In embodiments, the level of the biomarker is elevated by at least 50% relative to the control. In embodiments, the level of the biomarker is elevated by at least 55% relative to the control. In embodiments, the level of the biomarker is elevated by at least 60% relative to the control. In embodiments, the level of the biomarker is elevated by at least 65% relative to the control. In embodiments, the level of the biomarker is elevated by at least 70% relative to the control. In embodiments, the level of the biomarker is elevated by at least 75% relative to the control. In embodiments, the level of the biomarker is elevated by at least 80% relative to the control. In embodiments, the level of the biomarker is elevated by at least 85% relative to the control. In embodiments, the level of the biomarker is elevated by at least 90% relative to the control. In embodiments, the level of the biomarker is elevated by at least 95% relative to the control. In embodiments, the level of the biomarker is elevated by at least 100% relative to the control.

In embodiments, the level of the biomarker is elevated by at least 0.1× relative to the control. In embodiments, the level of the biomarker is elevated by at least 0.5× relative to the control. In embodiments, the level of the biomarker is elevated by at least 1× relative to the control. In embodiments, the level of the biomarker is elevated by at least 2× relative to the control. In embodiments, the level of the biomarker is elevated by at least 3× relative to the control. In embodiments, the level of the biomarker is elevated by at least 4× relative to the control. In embodiments, the level of the biomarker is elevated by at least 5× relative to the control. In embodiments, the level of the biomarker is elevated by at least 6× relative to the control. In embodiments, the level of the biomarker is elevated by at least 7× relative to the control. In embodiments, the level of the biomarker is elevated by at least 8× relative to the control. In embodiments, the level of the biomarker is elevated by at least 9× relative to the control. In embodiments, the level of the biomarker is elevated by at least 10× relative to the control. In embodiments, the level of the biomarker is elevated by at least 20× relative to the control. In embodiments, the level of the biomarker is elevated by at least 30× relative to the control. In embodiments, the level of the biomarker is elevated by at least 40× relative to the control. In embodiments, the level of the biomarker is elevated by at least 50× relative to the control. In embodiments, the level of the biomarker is elevated by at least 60× relative to the control. In embodiments, the level of the biomarker is elevated by at least 70× relative to the control. In embodiments, the level of the biomarker is elevated by at least 80× relative to the control. In embodiments, the level of the biomarker is elevated by at least 90× relative to the control. In embodiments, the level of the biomarker is elevated by at least 100× relative to the control. In embodiments, the level of the biomarker is elevated by at least 150× relative to the control. In embodiments, the level of the biomarker is elevated by at least 200× relative to the control.

In embodiments, the decreased level of a biomarker is a level that is lower than at least about 5%, at least about 10%, at least about 15%, at least about 20%, at least about 30%, at least about 40%, at least about 50%, at least about 60%, at least about 70%, at least about 80%, at least about 90%, or at least about 100% of the level of the biomarker in a control (e.g. the level of the biomarker from a cervical fluid sample of a subject without the pregnancy-associated risk or condition, the level of the biomarker from a cervical fluid sample of a subject who is not pregnant). In embodiments, the decreased level of a biomarker is a level that is lower than at least about 500%, at least about 1,000%, at least about 5,000%, or at least about 10,000% of a control (e.g. the level of the biomarker from a cervical fluid sample of a subject without the pregnancy-associated risk or condition, the level of the biomarker from a cervical fluid sample of a subject who is not pregnant).

In embodiments, the biomarker is decreased by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, or at most about 5% of a control (e.g. the level of the biomarker from a cervical fluid sample of a subject without the pregnancy-associated risk or condition, the level of the biomarker from a cervical fluid sample of a subject who is not pregnant).

In embodiments, the decreased level of a biomarker is lower than at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold of a control (e.g. the level of the biomarker from a cervical fluid sample of a subject without the pregnancy-associated risk or condition, the level of the biomarker from a cervical fluid sample of a subject who is not pregnant).

In embodiments, the biomarker is decreased by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold of a control (e.g. the level of the biomarker from a cervical fluid sample of a subject without the pregnancy-associated risk or condition, the level of the biomarker from a cervical fluid sample of a subject who is not pregnant).

In embodiments, the level of the biomarker is decreased by at least 5% relative to the control. In embodiments, the level of the biomarker is decreased by at least 10% relative to the control. In embodiments, the level of the biomarker is decreased by at least 15% relative to the control. In embodiments, the level of the biomarker is decreased by at least 20% relative to the control. In embodiments, the level of the biomarker is decreased by at least 25% relative to the control. In embodiments, the level of the biomarker is decreased by at least 30% relative to the control. In embodiments, the level of the biomarker is decreased by at least 35% relative to the control. In embodiments, the level of the biomarker is decreased by at least 40% relative to the control. In embodiments, the level of the biomarker is decreased by at least 45% relative to the control. In embodiments, the level of the biomarker is decreased by at least 50% relative to the control. In embodiments, the level of the biomarker is decreased by at least 55% relative to the control. In embodiments, the level of the biomarker is decreased by at least 60% relative to the control. In embodiments, the level of the biomarker is decreased by at least 65% relative to the control. In embodiments, the level of the biomarker is decreased by at least 70% relative to the control. In embodiments, the level of the biomarker is decreased by at least 75% relative to the control. In embodiments, the level of the biomarker is decreased by at least 80% relative to the control. In embodiments, the level of the biomarker is decreased by at least 85% relative to the control. In embodiments, the level of the biomarker is decreased by at least 90% relative to the control. In embodiments, the level of the biomarker is decreased by at least 95% relative to the control. In embodiments, the level of the biomarker is decreased by at least 100% relative to the control.

In embodiments, the level of the biomarker is decreased by at least 0.1× relative to the control. In embodiments, the level of the biomarker is decreased by at least 0.5× relative to the control. In embodiments, the level of the biomarker is decreased by at least 1× relative to the control. In embodiments, the level of the biomarker is decreased by at least 2× relative to the control. In embodiments, the level of the biomarker is decreased by at least 3× relative to the control. In embodiments, the level of the biomarker is decreased by at least 4× relative to the control. In embodiments, the level of the biomarker is decreased by at least 5× relative to the control. In embodiments, the level of the biomarker is decreased by at least 6× relative to the control. In embodiments, the level of the biomarker is decreased by at least 7× relative to the control. In embodiments, the level of the biomarker is decreased by at least 8× relative to the control. In embodiments, the level of the biomarker is decreased by at least 9× relative to the control. In embodiments, the level of the biomarker is decreased by at least 10× relative to the control. In embodiments, the level of the biomarker is decreased by at least 20× relative to the control. In embodiments, the level of the biomarker is decreased by at least 30× relative to the control. In embodiments, the level of the biomarker is decreased by at least 40× relative to the control. In embodiments, the level of the biomarker is decreased by at least 50× relative to the control. In embodiments, the level of the biomarker is decreased by at least 60× relative to the control. In embodiments, the level of the biomarker is decreased by at least 70× relative to the control. In embodiments, the level of the biomarker is decreased by at least 80× relative to the control. In embodiments, the level of the biomarker is decreased by at least 90× relative to the control. In embodiments, the level of the biomarker is decreased by at least 100× relative to the control. In embodiments, the level of the biomarker is decreased by at least 150× relative to the control. In embodiments, the level of the biomarker is decreased by at least 200× relative to the control.

In embodiments, detection of elevated levels or decreased levels of multiple biomarkers indicate an early gestational complication. Thus, for the method provided herein, in embodiments, the elevated level or decreased level of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, or at least 100 biomarkers can be detected. In embodiments, the elevated level or decreased level of at most 100, at most 90, at most 80, at most 70, at most 60, at most 50, at most 40, at most 30, at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, or at most 2 biomarkers can be detected.

In embodiments, the level of at least one biomarker is detected after isolating (e.g. removing) cells from the cervical fluid sample obtained from the subject. Thus, in embodiments, the method further includes isolating cells (e.g. living cells, dead cells, fragments of cells) from the cervical fluid sample between step a) and step b). In embodiments, isolating cells includes separating cells from the non-cellular components of the cervical fluid sample. Thus, in embodiments, isolating or removing the cells from the cervical fluid sample results in a cervical fluid sample including no greater than 1 cell per 1 mL volume. In embodiments, isolating or removing the cells from the cervical fluid sample results in a substantially cell free cervical fluid sample. Thus, in embodiments, the method of identifying a pregnancy-associated risk or condition includes a) obtaining a cervical fluid sample from a subject; b) isolating cells from the cervical fluid sample, thereby resulting in a cervical fluid sample including no greater than 1 cell per 1 mL volume, and c) detecting an elevated level or a decreased level of at least one biomarker in the cervical fluid sample including no greater than 1 cell per 1 mL volume relative to a standard control, thereby identifying the pregnancy associated risk or condition. In embodiments, the method of identifying a pregnancy-associated risk or condition includes a) obtaining a cervical fluid sample from a subject; b) isolating cells from the cervical fluid sample, thereby resulting in a substantially cell free cervical fluid sample, and c) detecting an elevated level or a decreased level of at least one biomarker in the substantially cell free cervical fluid sample relative to a standard control, thereby identifying the pregnancy associated risk or condition.

In embodiments, the method includes removing a cellular fraction from a non-cellular fraction of the cervical fluid sample obtained from the subject. Removal of cells from the cervical fluid sample can include any method well-known to those skilled in the art. For example, cells may be removed from the cervical fluid sample using methods including enzymatic methods, filtration, microfiltration, centrifugation, density-gradient centrifugation, fluorescence activated cell separation, or FAC, and magnetic activated cell sorting (MACS).

In embodiments, the method does not include isolating cells from the cervical fluid sample obtained from the subject. For example, in embodiments, detection of the elevated or decreased level of at least one biomarker in the cervical fluid sample relative to a standard control may occur without isolating (e.g. removing) cells from the cervical fluid sample. For example, the cervical fluid sample as originally obtained from the subject may include no greater than 1 cell per 1 mL volume. In another example, the cervical fluid sample as originally obtained from the subject may be cell free or substantially cell free.

In embodiments, the cervical fluid sample can be collected in a solution that contains a fixative. Alternatively, a sample can be collected in a solution that does not contain a fixative. As used herein, “fixative” refers to a composition used to prevent, slow, or inhibit the degradation of biomolecules (e.g. protein, DNA etc.) or cells, or to stabilize the biomolecules or cells.

In embodiments, the cervical fluid sample can undergo processing prior to analysis. In embodiments, and as described above, cells may be isolated (e.g. removed) from the cervical fluid sample. In embodiments, isolation of the cells from the cervical fluid sample results in a cervical fluid sample including no more than 1 cell per 1 mL volume (e.g. no more than 2 cells per 2 mL volume).

In embodiments, the cervical fluid sample can undergo separation and isolation of its constituent components. In embodiments, the cervical fluid sample can be washed or filtered to remove non-biological materials or contaminants. In embodiments, the cervical fluid sample can be diluted or concentrated. In embodiments, the cervical fluid sample can undergo one processing step, multiple processing steps, or no processing steps. In embodiments, the cervical fluid sample can explicitly avoid certain processing steps (e.g., isolation of components). In embodiments, the analysis is carried out on the cervical fluid sample without any intermediate processing steps.

Analysis of biomarkers produced by placenta-specific cells (e.g. EVT cells) are contemplated to be useful for assessing pregnancy status and assessing risk of pregnancy-related risks and conditions. Applicant has discovered that altered levels of proteins expressed by placenta-specific cells, fetal cells, or maternal cells can be detected in a cervical fluid sample (e.g. substantially cell-free cervical fluid sample) obtained from a subject (e.g. a pregnant subject). Placenta-specific cells may, for example, express extra-villous trophoblast (EVT) lineage markers (e.g., human chorionic gonadotropin (hCG) or human leukocyte antigen G (HLA-G)) and have molecular profiles associated with pathology. For example, altered levels of biomarkers including Alpha-fetoprotein (AFP) and placental growth factor (PGF) expressed by EVT cells are detectable in cervical fluid samples. In embodiments, altered levels of these biomarkers relative to a control are associated with pregnancy risk factors (e.g., FGR) and can be a key indicator of placenta-based perinatal disorders. In another example, altered levels of biomarkers expressed by fetal cells and found in cervical fluid samples are associated with fetal growth rates. In embodiments, detection of an elevated level or a decreased level of at least one biomarker expressed by EVT cells relative to a standard control is indicative of a pregnancy-associated risk or condition, for example, miscarriage, fetal growth restriction (FGR), preeclampsia.

“Placenta-specific cell” refers to a type of cell derived from the extra-embryonic tissues that creates the placenta of the fetus' or newborn's placental blood or tissue. Placenta-specific cells may include one of the cells that compose the three layers of the placenta. The first layer comprises trophoblast cells (e.g. extra-villous trophoblast cells), which are formed during the first stage of pregnancy and are the first cells to differentiate from the fertilized egg. The second layer comprises mesenchymal cells, mesenchymal derived macrophages, and fibroblasts. The third layer comprises fetal vascular cells, perivascular cells, and endothelial cells. Thus, for the method provided herein, in embodiments, the biomarker is derived from an EVT, a placental-derived mesenchymal cell, a placental-derived mesenchymal derived macrophage, or a placental-derived fibroblast. In embodiments, the biomarker is derived from a fetal vascular cell, a perivascular cell, or an endothelial cell.

For the methods provided herein, in embodiments, the placenta-specific cell is an extravillous trophoblast (EVT), villous trophoblast or syncytiotrophoblast cell. In embodiments, the placenta-specific cell is an EVT. Extra-villous trophoblast cells, also known as cervical EVTs (cEVTs), are cells which originate from the distal side of trophoblast cell columns derived from cytotrophoblasts of anchoring placental villi. EVTs enable the placenta to attach to the uterus and allow for a fetus to obtain maternal nutrients. EVTs can reside in and/or pass through the cervical canal. In embodiments, altered levels of biomarkers derived from EVTs and detectable in cervical fluid samples are indicative of one or more pregnancy associated risks. Thus, in embodiments, the placenta-specific cell is a villous trophoblast. In embodiments, the placenta-specific cell is a syncytiotrophoblast cell.

In embodiments, the at least one biomarker is derived from a placenta-specific cell, a maternal cell, or a fetal cell. In embodiments, the at least one biomarker is derived from a placenta-specific cell. In embodiments, the at least one biomarker is derived from a maternal cell. In embodiments, the at least one biomarker is derived from a fetal cell.

Maternal cell refers to a cell derived from the subject (e.g. pregnant subject). In embodiments, maternal cells may circulate through and/or contact the placenta during pregnancy. Fetal cell refers to cell derived from an embryo or a fetus. In embodiments, fetal cells may circulate in maternal blood (e.g. fetalmaternal transfer). In embodiments, fetal cells (e.g. fetal mesenchymal cells) circulate throughout the placenta. Thus, in embodiments, a fetal cell may be derived from the placenta. Maternal cells, fetal cells, and biomarkers derived thereof may accumulate in the cervix, and thereby be detectable using the methods provided herein including embodiments thereof.

In embodiments, the biomarker is a pregnancy-associated biomarker, placental-related biomarker, EVT cell biomarker, or a cervical health related biomarker. A “pregnancy associated biomarker” is a biomolecule wherein the change or detection of an increased or decreased level of the biomolecule relative to a control is indicative of a pregnancy-associated risk, condition, or state. In embodiments, the change of the level of a pregnancy associated biomarker is indicative of a condition or state of the embryo or fetus. Biomarkers (e.g. pregnancy-associated biomarkers) include human chorionic gonadotropin (hCG) and progesterone. Other pregnancy-associated biomarkers include alpha fetoprotein (AFP), placental growth factor (PGF or PLGF), pregnancy-associated plasma protein A (PAPPA), and major histocompatibility complex, class I, G (HLA-G).

For the methods provided herein, in embodiments, the at least one biomarker is a protein, nucleic acid, cell fragment, microvesicle, ectosome, microparticle, extracellular vesicle, micelle, or combination thereof. In embodiments, the at least one biomarker is a protein. In embodiments, the at least one biomarker is a nucleic acid. The nucleic acid may encode any one of the proteins provided herein including embodiments thereof. In embodiments, the nucleic acid includes a gene or a fragment of a gene encoding any one of the proteins provided herein including embodiments thereof. In one example, nucleic acids encoding genes associated with pregnancy-associated risks may be detected in the cervical fluid sample using methods including PCR. In another example, nucleic acids associated with fetal characteristics (e.g. sex, chromosomal abnormalities, etc.) may be detected in the cervical fluid sample. In embodiments, the at least one biomarker is a cell fragment. In embodiments, the at least one biomarker is a microvesicle. In embodiments, the at least one biomarker is an ectosome. In embodiments, the at least one biomarker is a microparticle. In embodiments, the at least one biomarker is an extracellular vesicle. In embodiments, the at least one biomarker is a micelle. In embodiments, the one or more biomarkers may include a metabolite associated with a pregnancy-associated risk or condition, including glucose, cholesterol, or a saturated fatty acid. Micelles, ectosomes and extracellular vesicles may be found in the soluble fraction or non-cellular fraction of the cervical fluid sample provided herein. In one example, micelles, ectosomes and extracellular vesicles may encapsulate biomarkers (e.g. DNA, proteins, etc.) associated with pregnancy-related risks.

For the method provided herein, in embodiments, the biomarker is a protein. In embodiments, the protein is placental growth factor (PGF), pregnancy-associated plasma protein-A (PAPP-A), galectin 13 (LGALS13), galectin 14 (LGALS14), alpha fetoprotein (AFP), endoglin (ENG), fms-like tyrosine kinase 1 (FLT), CGB, ADAM12, ADAM17, BDNF, CCL5, CRP, CXCL8, EGF, SEGFR, EPO, HBEGF, IFNG, IGF, IGFBP1, ILIB, IL6, INHA, MMP2, MMP7, MMP9, MMP12, NGF, TGA1, TGFA, TGFB2, TIMP3, TNFA, TSH, VEGF, or fragments or combinations thereof. In embodiments, the protein is PGF, PAPP-A, LGALS13, LGALS14, AFP, ENG, FLT, or a fragment or combination thereof. In embodiments, the protein is placental growth factor (PGF) or a fragment thereof. In embodiments, the protein is pregnancy-associated plasma protein-A (PAPP-A) or a fragment thereof. In embodiments, the protein is galectin 13 (LGALS13) or a fragment thereof. In embodiments, the protein is galectin 14 (LGALS14) or a fragment thereof. In embodiments, the protein is alpha fetoprotein (AFP) or a fragment thereof. In embodiments, the protein is endoglin (ENG) or a fragment thereof. In embodiments, the protein is fms-like tyrosine kinase 1 (FLT) or a fragment thereof. In embodiments, the protein is CGB or a fragment thereof. In embodiments, the protein is ADAM12 or a fragment thereof. In embodiments, the protein is ADAM17 or a fragment thereof. In embodiments, the protein is BDNF or a fragment thereof. In embodiments, the protein is CCL5 or a fragment thereof. In embodiments, the protein is CRP or a fragment thereof. In embodiments, the protein is CXCL8 or a fragment thereof. In embodiments, the protein is EGF or a fragment thereof. In embodiments, the protein is sEGFR or a fragment thereof. In embodiments, the protein is EPO or a fragment thereof. In embodiments, the protein is HBEGF or a fragment thereof. In embodiments, the protein IFNG or a fragment thereof. In embodiments, the protein is IGF or a fragment thereof In embodiments, the protein is IGFBP1 or a fragment thereof In embodiments, the protein is ILIB or a fragment thereof. In embodiments, the protein is IL6 or a fragment thereof. In embodiments, the protein is INHA or a fragment thereof. In embodiments, the protein is MMP2 or a fragment thereof. In embodiments, the protein is MMP7 or a fragment thereof. In embodiments, the protein is MMP9 or a fragment thereof. In embodiments, the protein is MMP12 or a fragment thereof. In embodiments, the protein is NGF or a fragment thereof. In embodiments, the protein is TGA1 or a fragment thereof. In embodiments, the protein is TGFA or a fragment thereof. In embodiments, the protein is TGFB2 or a fragment thereof. In embodiments, the protein is TIMP3 or a fragment thereof. In embodiments, the protein is TNFA or a fragment thereof. In embodiments, the protein TSH or a fragment thereof. In embodiments, the protein is VEGF or a fragment thereof.

In embodiments, the at least one biomarker includes an EVT biomarker. The term “EVT biomarker” refers to a compound (e.g. a protein, nucleic acid, etc.) expressed by or produced by an extravillous trophoblast (EVT). In embodiments, an elevated level or decreased level of an EVT biomarker relative to a control is indicative of a pregnancy-associated risk or condition. In embodiments, the EVT biomarker includes integrin subunit alpha 1 (ITGA1), cadherin 5 (CDH5), cadherin 1 (CDH1), platelet and endothelial cell adhesion (PECAM1), matrix metallopeptidase 9 (MMP9), HLA-G, integrin alpha 6 (ITGA6), chorionic gonadotropin (hCG), pregnancy-specific beta-1-glycoprotein 1 (PSG1) or a fragment or combination thereof. In embodiments, the EVT biomarker is integrin subunit alpha 1 (ITGA1) or a fragment thereof. In embodiments, the EVT biomarker is cadherin 5 (CDH5) or a fragment thereof. In embodiments, the EVT biomarker is cadherin 1 (CDH1) or a fragment thereof. In embodiments, the EVT biomarker is platelet and endothelial cell adhesion (PECAM1) or a fragment thereof. In embodiments, the EVT biomarker is matrix metallopeptidase 9 (MMP9) or a fragment thereof. In embodiments, the EVT biomarker is HLA-G or a fragment thereof. In embodiments, the EVT biomarker is integrin alpha 6 (ITGA6) or a fragment thereof. In embodiments, the EVT biomarker is chorionic gonadotropin (hCG) or a fragment thereof. In embodiments, the EVT biomarker is pregnancy-specific beta-1-glycoprotein 1 (PSG1) or a fragment thereof. In embodiments, the EVT biomarker is laeverin or a fragment thereof.

In embodiments, the at least one biomarker includes a placental protein. “Placental protein” refers to a protein or fragment thereof derived from placental tissue or a placenta-specific cell, wherein detection of an elevated level or decreased level of the protein is associated with a pregnancy-associated condition or risk. For example, a placental protein may be expressed by a placenta-specific cell. In embodiments, detection of an elevated level or decreased level of the placental protein is associated with a condition or state of the embryo or fetus. In embodiments, placental proteins are derived from placenta-specific cells. For example, the placental protein may be derived from a syncytial trophoblast. Syncytial trophoblast typically express placenta-specific growth factors and hormones including biomarkers associated with placental growth and health. Placental proteins expressed by Syncytial trophoblasts include, for example, hCG, progesterone, human placental lactogen, vascular endothelial growth factor (VEGF), VEGF-receptor (FLT1), PGF, insulin-like growth factor binding protein 1 (IGFBP1 or PP12), galectin 13 (LGALS13), galectin 14 (LGALS14), vasculotropin, PAPPA, endoglin (ENG), and vascular endothelial cell proliferation factor and fragments thereof. The mammalian placental-specific galectins PP12, LGALS13, and LGALS14 in particular can decrease significantly in maternal serum during the first trimester in women who later developed early-onset PE and FGR.

In embodiments, the placental protein includes hCG, progesterone, human placental lactogen, vascular endothelial growth factor (VEGF), VEGF-receptor (FLT1), PGF, insulin-like growth factor binding protein 1 (IGFBP1 or PP12), galectin 13 (LGALS13), galectin 14 (LGALS14), vasculotropin, PAPPA, endoglin (ENG), vascular endothelial cell proliferation factor, or a fragment or combination thereof. In embodiments, the placental protein includes hCG or a fragment thereof. In embodiments, the placental protein includes progesterone or a fragment thereof. In embodiments, the placental protein includes human placental lactogen or a fragment thereof. In embodiments, the placental protein includes vascular endothelial growth factor (VEGF) or a fragment thereof. In embodiments, the placental protein VEGF-receptor (FLT1) or a fragment thereof. In embodiments, the placental protein includes PGF or a fragment thereof. In embodiments, the placental protein includes insulin-like growth factor binding protein 1 (IGFBP1 or PP12) or a fragment thereof. In embodiments, the placental protein includes galectin 13 (LGALS13) or a fragment thereof. In embodiments, the placental protein includes galectin 14 (LGALS14) or a fragment thereof. In embodiments, the placental protein includes vasculotropin or a fragment thereof. In embodiments, the placental protein includes PAPPA or a fragment thereof. In embodiments, the placental protein includes endoglin (ENG) or a fragment thereof. In embodiments, the placental protein includes vascular endothelial cell proliferation factor or a fragment thereof.

Placental growth factor (PGF or PLGF) has pro-angiogenic effects on the feto-placental circulation and supports trophoblast growth. Low circulating PGF precedes the manifestation of clinical disease in pre-eclamptic pregnancies and intrauterine growth restriction. The term “placental growth factor protein” or “placental growth factor” as used herein includes any of the recombinant or naturally-occurring forms of placental growth factor (PGF), also known as PGFL, PLGF, or variants or homologs thereof that maintain PGF activity (e.g. within at least 50%, 80%, 90%, 95%, 96%, 97%, 98%, 99% or 100% activity compared to PGF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PGF protein In embodiments, the PGF protein is substantially identical to the protein identified by the UniProt reference number Q01974 or a variant or homolog having substantial identity thereto.

The term “pregnancy-associated plasma protein-A protein” or “pregnancy-associated plasma protein-A” as used herein includes any of the recombinant or naturally-occurring forms of pregnancy-associated plasma protein-A (PAPP-A), also known as PAPPA, Pappalysin-1, insulin-like growth factor-dependent IGF-binding protein 4 protease, IGF-dependent IGFBP-4 protease, or variants or homologs thereof that maintain PAPP-A activity (e.g. within at least 50%, 80%, 90%, 95%, 96%, 97%, 98%, 99% or 100% activity compared to PAPP-A). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PAPP-A protein In embodiments, the PAPP-A protein is substantially identical to the protein identified by the UniProt reference number Q13219 or a variant or homolog having substantial identity thereto.

The term “galectin 14 protein” or “galectin 14” as used herein includes any of the recombinant or naturally-occurring forms of galectin 14 (LGALS14), also known as Placental protein 13-like, Gal-14, Charcot-Leyden crystal protein 2 or variants or homologs thereof that maintain LGALS14 activity (e.g. within at least 50%, 80%, 90%, 95%, 96%, 97%, 98%, 99% or 100% activity compared to LGALS14). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LGALS14 protein In embodiments, the LGALS14 protein is substantially identical to the protein identified by the UniProt reference number Q8TCE9 or a variant or homolog having substantial identity thereto.

Alpha fetoprotein (AFP) is a plasma protein produced by the embryonic yolk sac and the fetal liver. AFP levels in serum, amniotic fluid, and urine can be used in screening tests for congenital disabilities, chromosomal abnormalities, as well as some other adult occurring tumors and pathologies. The term “alpha fetoprotein” or “alpha fetoprotein protein” as used herein includes any of the recombinant or naturally-occurring forms of alpha fetoprotein (AFP), also known AS Alpha-1-fetoprotein, or variants or homologs thereof that maintain AFP activity (e.g. within at least 50%, 80%, 90%, 95%, 96%, 97%, 98%, 99% or 100% activity compared to AFP). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring AFP protein In embodiments, the AFP protein is substantially identical to the protein identified by the UniProt reference number P02771 or a variant or homolog having substantial identity thereto.

The term “endoglin” or “endoglin protein” as used herein includes any of the recombinant or naturally-occurring forms of endoglin (ENG), also known as CD105, or variants or homologs thereof that maintain ENG activity (e.g. within at least 50%, 80%, 90%, 95%, 96%, 97%, 98%, 99% or 100% activity compared to ENG). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ENG protein In embodiments, the ENG protein is substantially identical to the protein identified by the UniProt reference number P17813 or a variant or homolog having substantial identity thereto.

The term “Fms-like tyrosine kinase 1” or “Fms-like tyrosine kinase 1 protein” as used herein includes any of the recombinant or naturally-occurring forms of Fms-like tyrosine kinase 1 (FLT), also known as soluble Fms-like tyrosine kinase 1, sFLT, Vascular endothelial growth factor receptor 1, VEGFR-1, Tyrosine-protein kinase FRT or variants or homologs thereof that maintain FLT activity (e.g. within at least 50%, 80%, 90%, 95%, 96%, 97%, 98%, 99% or 100% activity compared to FLT). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring FLT protein In embodiments, the FLT protein is substantially identical to the protein identified by the UniProt reference number P17948 or a variant or homolog having substantial identity thereto.

Human chorionic gonadotropin (hCG, HCG, CGB) is a hormone produced by the placenta during pregnancy. HCG assists in thickening the uterine lining to support a growing embryo. HCG is further involved in signaling processes resulting in cessation of menstruation during pregnancy. In embodiments, HCG levels increase following conception and continue to increase until about 10 weeks in pregnancy. The term “CGB protein” or “CGB” as used herein includes any of the recombinant or naturally-occurring forms of Choriogonadotropin subunit beta 3 (CBG), also known as chorionic gonadotropin chain beta, chorionic gonadotropin, or variants or homologs thereof that maintain CGB activity (e.g. within at least 50%, 80%, 90%, 95%, 96%, 97%, 98%, 99% or 100% activity compared to CGB). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CGB protein In embodiments, the CGB protein is substantially identical to the protein identified by the UniProt reference number P0DN86 or a variant or homolog having substantial identity thereto.

The term “ADAM12 protein” or “ADAM12” as used herein includes any of the recombinant or naturally-occurring forms of Disintegrin and metalloproteinase domain-containing protein 12 (ADAM12), or variants or homologs thereof that maintain ADAM12 activity (e.g. within at least 50%, 80%, 90%, 95%, 96%, 97%, 98%, 99% or 100% activity compared to ADAM12). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ADAM12 protein In embodiments, the ADAM12 protein is substantially identical to the protein identified by the UniProt reference number 043184 or a variant or homolog having substantial identity thereto.

The term “ADAM17 protein” or “ADAM17” as used herein includes any of the recombinant or naturally-occurring forms of Disintegrin and metalloproteinase domain-containing protein 17 (ADAM17), also known as TNF-alpha-converting enzyme, CD156b, or variants or homologs thereof that maintain ADAM17 activity (e.g. within at least 50%, 80%, 90%, 95%, 96%, 97%, 98%, 99% or 100% activity compared to ADAM17). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ADAM17 protein In embodiments, the ADAM17 protein is substantially identical to the protein identified by the UniProt reference number P78536 or a variant or homolog having substantial identity thereto.

The term “BDNF protein” or “BDNF” as used herein includes any of the recombinant or naturally-occurring forms of Brain-derived neurotrophic factor (BDNF), or variants or homologs thereof that maintain BDNF activity (e.g. within at least 50%, 80%, 90%, 95%, 96%, 97%, 98%, 99% or 100% activity compared to BDNF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring BDNF protein In embodiments, the BDNF protein is substantially identical to the protein identified by the UniProt reference number P23560 or a variant or homolog having substantial identity thereto.

The term “CCL5 protein” or “CCL5” as used herein includes any of the recombinant or naturally-occurring forms of C—C motif chemokine 5 (CCL5), or variants or homologs thereof that maintain CCL5 activity (e.g. within at least 50%, 80%, 90%, 95%, 96%, 97%, 98%, 99% or 100% activity compared to CCL5). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CCL5 protein In embodiments, the CCL5 protein is substantially identical to the protein identified by the UniProt reference number P13501 or a variant or homolog having substantial identity thereto.

The term “CRP protein” or “CRP” as used herein includes any of the recombinant or naturally-occurring forms of C-reactive protein (CRP), or variants or homologs thereof that maintain CRP activity (e.g. within at least 50%, 80%, 90%, 95%, 96%, 97%, 98%, 99% or 100% activity compared to CRP). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CRP protein In embodiments, the CRP protein is substantially identical to the protein identified by the UniProt reference number P02741 or a variant or homolog having substantial identity thereto.

The term “CXCL8 protein” or “CXCL8” as used herein includes any of the recombinant or naturally-occurring forms of C—X—C motif chemokine 8 (CXCL8), also known as IL-8, Chemokine (C—X—C motif) ligand 8, or variants or homologs thereof that maintain CXCL8 activity (e.g. within at least 50%, 80%, 90%, 95%, 96%, 97%, 98%, 99% or 100% activity compared to CXCL8). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CXCL8 protein In embodiments, the CXCL8 protein is substantially identical to the protein identified by the UniProt reference number P10145 or a variant or homolog having substantial identity thereto.

The term “EGF protein” or “EGF” as used herein includes any of the recombinant or naturally-occurring forms of Pro-epidermal growth factor (EGF), or variants or homologs thereof that maintain EGF activity (e.g. within at least 50%, 80%, 90%, 95%, 96%, 97%, 98%, 99% or 100% activity compared to EGF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGF protein In embodiments, the EGF protein is substantially identical to the protein identified by the UniProt reference number P01133 or a variant or homolog having substantial identity thereto.

The term “EGFR protein” or “EGFR” as used herein includes any of the recombinant or naturally-occurring forms of Epidermal growth factor receptor (EGFR), or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein In embodiments, the EGFR protein is substantially identical to the protein identified by the UniProt reference number P0CY46 or a variant or homolog having substantial identity thereto.

The term “Erythropoietin protein” or “Erythropoictin” as used herein includes any of the recombinant or naturally-occurring forms of Erythropoietin (EPO), or variants or homologs thereof that maintain EPO activity (e.g. within at least 50%, 80%, 90%, 95%, 96%, 97%, 98%, 99% or 100% activity compared to EPO). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EPO protein In embodiments, the EPO protein is substantially identical to the protein identified by the UniProt reference number P01588 or a variant or homolog having substantial identity thereto.

The term “IFNG protein” or “IFNG” as used herein includes any of the recombinant or naturally-occurring forms of Interferon gamma (INFG), or variants or homologs thereof that maintain INFG activity (e.g. within at least 50%, 80%, 90%, 95%, 96%, 97%, 98%, 99% or 100% activity compared to INFG). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring INFG protein In embodiments, the INFG protein is substantially identical to the protein identified by the UniProt reference number P01579 or a variant or homolog having substantial identity thereto.

The term “HBEGF protein” or “HBEGF” as used herein includes any of the recombinant or naturally-occurring forms of Proheparin-binding EGF-like growth factor (HBEGF), or variants or homologs thereof that maintain HBEGF activity (e.g. within at least 50%, 80%, 90%, 95%, 96%, 97%, 98%, 99% or 100% activity compared to HBEGF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HBEGF protein In embodiments, the HBEGF protein is substantially identical to the protein identified by the UniProt reference number Q99075 or a variant or homolog having substantial identity thereto.

The term “Insulin-like growth factor I protein” or “Insulin-like growth factor I” as used herein includes any of the recombinant or naturally-occurring forms of Insulin-like growth factor I (IGF), or variants or homologs thereof that maintain IGF activity (e.g. within at least 50%, 80%, 90%, 95%, 96%, 97%, 98%, 99% or 100% activity compared to IGF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IGF protein In embodiments, the IGF protein is substantially identical to the protein identified by the UniProt reference number P05019 or a variant or homolog having substantial identity thereto.

The term “Insulin-like growth factor-binding I protein” or “Insulin-like growth factor-binding I protein” as used herein includes any of the recombinant or naturally-occurring forms of Insulin-like growth factor I (IGFBP1), or variants or homologs thereof that maintain IGFBP1 activity (e.g. within at least 50%, 80%, 90%, 95%, 96%, 97%, 98%, 99% or 100% activity compared to IGFBP1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IGFBP1 protein In embodiments, the IGFBP1 protein is substantially identical to the protein identified by the UniProt reference number P08833 or a variant or homolog having substantial identity thereto.

The term “Interleukin-1 beta protein” or “Interleukin-1 beta” as used herein includes any of the recombinant or naturally-occurring forms of Insulin-like growth factor I (IL1B), or variants or homologs thereof that maintain ILIB activity (e.g. within at least 50%, 80%, 90%, 95%, 96%, 97%, 98%, 99% or 100% activity compared to IL1B). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ILIB protein In embodiments, the ILIB protein is substantially identical to the protein identified by the UniProt reference number P01584 or a variant or homolog having substantial identity thereto.

The term “Interleukin-6 protein” or “Interleukin-6” as used herein includes any of the recombinant or naturally-occurring forms of Interleukin-6 (IL6), or variants or homologs thereof that maintain IL6 activity (e.g. within at least 50%, 80%, 90%, 95%, 96%, 97%, 98%, 99% or 100% activity compared to IL6). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL6 protein In embodiments, the IL6 protein is substantially identical to the protein identified by the UniProt reference number P05231 or a variant or homolog having substantial identity thereto.

The term “Inhibin alpha chain protein” or “Inhibin alpha chain” as used herein includes any of the recombinant or naturally-occurring forms of Inhibin alpha chain (INHA), or variants or homologs thereof that maintain INHA activity (e.g. within at least 50%, 80%, 90%, 95%, 96%, 97%, 98%, 99% or 100% activity compared to INHA). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring INHA protein In embodiments, the INHA protein is substantially identical to the protein identified by the UniProt reference number P05111 or a variant or homolog having substantial identity thereto.

The term “MMP2 protein” or “MMP2” as used herein includes any of the recombinant or naturally-occurring forms of Matrix metalloproteinase-2 (MMP2), also known as 72 kDa type IV collagenase, Gelatinase A, or variants or homologs thereof that maintain MMP2 activity (e.g. within at least 50%, 80%, 90%, 95%, 96%, 97%, 98%, 99% or 100% activity compared to MMP2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MMP2 protein In embodiments, the MMP2 protein is substantially identical to the protein identified by the UniProt reference number P08253 or a variant or homolog having substantial identity thereto.

The term “MMP7 protein” or “MMP7” as used herein includes any of the recombinant or naturally-occurring forms of Matrix metalloproteinase-7 (MMP7), also known as Uterine metalloproteinase, or variants or homologs thereof that maintain MMP7 activity (e.g. within at least 50%, 80%, 90%, 95%, 96%, 97%, 98%, 99% or 100% activity compared to MMP7). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MMP7 protein In embodiments, the MMP7 protein is substantially identical to the protein identified by the UniProt reference number P09237 or a variant or homolog having substantial identity thereto.

The term “MMP9 protein” or “MMP9” as used herein includes any of the recombinant or naturally-occurring forms of Matrix metalloproteinase-9 (MMP9), also known as gelatinase B, or variants or homologs thereof that maintain MMP9 activity (e.g. within at least 50%, 80%, 90%, 95%, 96%, 97%, 98%, 99% or 100% activity compared to MMP9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MMP9 protein In embodiments, the MMP9 protein is substantially identical to the protein identified by the UniProt reference number P14780 or a variant or homolog having substantial identity thereto.

The term “MMP12 protein” or “MMP12” as used herein Macrophage metalloelastase (MMP12), or variants or homologs thereof that maintain MMP12 activity (e.g. within at least 50%, 80%, 90%, 95%, 96%, 97%, 98%, 99% or 100% activity compared to MMP12). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MMP12 protein In embodiments, the MMP12 protein is substantially identical to the protein identified by the UniProt reference number P39900 or a variant or homolog having substantial identity thereto.

The term “nerve growth factor protein” or “nerve growth factor” as used herein includes any of the recombinant or naturally-occurring forms of nerve growth factor (NGF), also known as gelatinase B, or variants or homologs thereof that maintain NGF activity (e.g. within at least 50%, 80%, 90%, 95%, 96%, 97%, 98%, 99% or 100% activity compared to NGF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring NGF protein In embodiments, the NGF protein is substantially identical to the protein identified by the UniProt reference number P01138 or a variant or homolog having substantial identity thereto.

The term “TGFA protein” or “TGFA” as used herein includes any of the recombinant or naturally-occurring forms of Protransforming growth factor alpha (TGFA), or variants or homologs thereof that maintain TGFA activity (e.g. within at least 50%, 80%, 90%, 95%, 96%, 97%, 98%, 99% or 100% activity compared to TGFA). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TGFA protein In embodiments, the TGFA protein is substantially identical to the protein identified by the UniProt reference number P01135 or a variant or homolog having substantial identity thereto.

The term “TIMP3 protein” or “TIMP3” as used herein includes any of the recombinant or naturally-occurring forms of Metalloproteinase inhibitor 3 (TIMP3), or variants or homologs thereof that maintain TIMP3 activity (e.g. within at least 50%, 80%, 90%, 95%, 96%, 97%, 98%, 99% or 100% activity compared to TIMP3). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TIMP3 protein In embodiments, the TIMP3 protein is substantially identical to the protein identified by the UniProt reference number P35625 or a variant or homolog having substantial identity thereto.

The term “thyroid stimulating hormone protein” or “thyroid stimulating hormone” as used herein includes any of the recombinant or naturally-occurring forms of thyroid stimulating hormone (TSH), also known as Glycoprotein hormones alpha chain, Choriogonadotropin alpha chain, Follicle-stimulating hormone alpha chain or variants or homologs thereof that maintain TSH activity (e.g. within at least 50%, 80%, 90%, 95%, 96%, 97%, 98%, 99% or 100% activity compared to TSH). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TSH protein In embodiments, the TSH protein is substantially identical to the protein identified by the UniProt reference number P01215 or a variant or homolog having substantial identity thereto.

The term “TGFB2 protein” or “TGFB2” as used herein includes any of the recombinant or naturally-occurring forms of thyroid stimulating hormone (TGFB2), or variants or homologs thereof that maintain TGFB2 activity (e.g. within at least 50%, 80%, 90%, 95%, 96%, 97%, 98%, 99% or 100% activity compared to TGFB2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TGFB2 protein In embodiments, the TGFB2 protein is substantially identical to the protein identified by the UniProt reference number P61812 or a variant or homolog having substantial identity thereto.

The term “TGFA protein” or “TGFA” as used herein includes any of the recombinant or naturally-occurring forms of tumor necrosis factor (TGFA), also known as tumor necrosis factor alpha, or variants or homologs thereof that maintain TGFA activity (e.g. within at least 50%, 80%, 90%, 95%, 96%, 97%, 98%, 99% or 100% activity compared to TGFA). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TGFA protein In embodiments, the TGFA protein is substantially identical to the protein identified by the UniProt reference number P01375 or a variant or homolog having substantial identity thereto.

The term “VEGF protein” or “VEGF” as used herein includes any of the recombinant or naturally-occurring forms of vascular endothelial growth factor (VEGF), or variants or homologs thereof that maintain VEGF activity (e.g. within at least 50%, 80%, 90%, 95%, 96%, 97%, 98%, 99% or 100% activity compared to VEGF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VEGF protein In embodiments, the VEGF protein is substantially identical to the protein identified by the UniProt reference number P15692 or a variant or homolog having substantial identity thereto.

The term “ITGA1 protein” or “ITGA1” as used herein includes any of the recombinant or naturally-occurring forms of integrin alpha 1 (ITGA1), also known as CD49a, or variants or homologs thereof that maintain ITGA1 activity (e.g. within at least 50%, 80%, 90%, 95%, 96%, 97%, 98%, 99% or 100% activity compared to ITGA1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ITGA1 protein In embodiments, the ITGA1 protein is substantially identical to the protein identified by the UniProt reference number P56199 or a variant or homolog having substantial identity thereto.

The term “cadherin 5 protein” or “cadherin 5” as used herein includes any of the recombinant or naturally-occurring forms of cadherin 5, or variants or homologs thereof that maintain cadherin 5 activity (e.g. within at least 50%, 80%, 90%, 95%, 96%, 97%, 98%, 99% or 100% activity compared to cadherin 5). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cadherin 5 protein In embodiments, the cadherin 5 protein is substantially identical to the protein identified by the UniProt reference number P33151 or a variant or homolog having substantial identity thereto.

The term “platelet and endothelial cell adhesion protein” or “platelet and endothelial cell adhesion” as used herein includes any of the recombinant or naturally-occurring forms of platelet and endothelial cell adhesion (PECAM1), or variants or homologs thereof that maintain PECAM1 activity (e.g. within at least 50%, 80%, 90%, 95%, 96%, 97%, 98%, 99% or 100% activity compared to PECAM1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PECAM1 protein In embodiments, the PECAM1 protein is substantially identical to the protein identified by the UniProt reference number P16284 or a variant or homolog having substantial identity thereto.

The term “HLA-G protein” or “HLA-G” as used herein includes any of the recombinant or naturally-occurring forms of HLA-G, also known as HLA class I histocompatibility antigen, alpha chain G or variants or homologs thereof that maintain HLA-G activity (e.g. within at least 50%, 80%, 90%, 95%, 96%, 97%, 98%, 99% or 100% activity compared to HLA-G). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HLA-G protein In embodiments, the HLA-G protein is substantially identical to the protein identified by the UniProt reference number P17693 or a variant or homolog having substantial identity thereto.

The term “PSG1 protein” or “PSG1” as used herein includes any of the recombinant or naturally-occurring forms of Pregnancy-specific beta-1-glycoprotein 1 (PSG1), variants or homologs thereof that maintain ITGA6 activity (e.g. within at least 50%, 80%, 90%, 95%, 96%, 97%, 98%, 99% or 100% activity compared to PSG1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PSG1 protein In embodiments, the PSG1 protein is substantially identical to the protein identified by the UniProt reference number P11464 or a variant or homolog having substantial identity thereto.

The term “ITGA6 protein” or “ITGA6” as used herein includes any of the recombinant or naturally-occurring forms of integrin alpha 6 (ITGA6), also known as CD49 antigen-like family member F or variants or homologs thereof that maintain ITGA6 activity (e.g. within at least 50%, 80%, 90%, 95%, 96%, 97%, 98%, 99% or 100% activity compared to ITGA6). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ITGA6 protein In embodiments, the ITGA6 protein is substantially identical to the protein identified by the UniProt reference number P23229 or a variant or homolog having substantial identity thereto.

The term “human placental lactogen protein” or “human placental lactogen” as used herein includes any of the recombinant or naturally-occurring forms of human placental lactogen, also known Chorionic somatomammotropin hormone 1, or variants or homologs thereof that maintain human placental lactogen activity (e.g. within at least 50%, 80%, 90%, 95%, 96%, 97%, 98%, 99% or 100% activity compared to human placental lactogen). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring human placental lactogen protein In embodiments, the human placental lactogen protein is substantially identical to the protein identified by the UniProt reference number Q6PF11 or a variant or homolog having substantial identity thereto.

For the methods provided herein, in embodiments, the detecting includes performing enzyme-linked immunosorbent assay (ELISA), gel electrophoresis, western blotting, mass spectrometry, capillary electrophoresis, protein sequencing, polymerase chain reaction (PCR), digital PCR, DNA sequencing using capillary electrophoresis or next-generation sequencing (NGS), reverse transcription of RNA followed by PCR, digital PCR or NGS, liquid chromatography, thin layer chromatography, or a combination thereof. In embodiments, the detecting includes performing an immunoassay, mass spectrometry, a polymerase chain reaction (PCR), a sequencing method, or a combination thereof. In embodiments, the detecting includes performing an immunoassay. In embodiments, the detecting includes performing mass spectrometry. In embodiments, the detecting includes performing PCR. In embodiments, the detecting includes performing a sequencing method. For the methods provided herein, in embodiments, the detecting includes an antibody-based method. In embodiments, the detecting may include performing a lateral flow assay.

In embodiments, the method further includes identifying a cervical condition. In embodiments, identifying the cervical condition includes determining an elevated level or a decreased level of at least one cervical health biomarker relative to a standard control. As used herein, “cervical health biomarker” refers to a compound the level of which is associated with a cervical condition. The cervical health biomarker may be a biomolecule (e.g. protein, DNA, metabolite, hormone, etc.) produced by a cell originating in the cervix. The cervical health biomarker may be a biomolecule derived from a maternal cell, fetal cell, or placenta-specific cell and accumulated in the cervical fluid sample. In embodiments, the cervical condition may be a cervical infection, bleeding, inflammation, or cervical cancer.

For the methods provided herein, in embodiments, the cervical health biomarker is a protein. In embodiments, the cervical health biomarker includes carcinoembryonic antigen (CEA), squamous cell carcinoma antigen (SCC Ag) and carbohydrate antigen 19-9 (CA19-9), HCGB, KRT7, hPL, CDH5, PECAM1, ITGA1, MMP9, TGFB2, HLAG, PSG1, ITGA6, CDH1, LGALS13, LGALS14, PAPPA, PGF, AFP, FLT, ENG, Spint1, ADAM12, MPO, CD68, BMK13, CD45, or a fragment or combination thereof. In embodiments, the cervical health biomarker is carcinoembryonic antigen (CEA) or a fragment thereof. In embodiments, the cervical health biomarker is squamous cell carcinoma antigen (SCC Ag) or a fragment thereof. In embodiments, the cervical health biomarker is and carbohydrate antigen 19-9 (CA19-9) or a fragment thereof. In embodiments, the cervical health biomarker is KRT7 or a fragment thereof. In embodiments, the cervical health biomarker is hPL or a fragment thereof. In embodiments, the cervical health biomarker CDH5 or a fragment thereof. In embodiments, the cervical health biomarker is PECAM1 or a fragment thereof. In embodiments, the cervical health biomarker is ITGA1 or a fragment thereof. In embodiments, the cervical health biomarker is MMP9 or a fragment thereof. In embodiments, the cervical health biomarker is TGFB2 or a fragment thereof. In embodiments, the cervical health biomarker is HLAG or a fragment thereof. In embodiments, the cervical health biomarker is PSG1 or a fragment thereof. In embodiments, the cervical health biomarker is ITGA6 or a fragment thereof. In embodiments, the cervical health biomarker is CDH1 or a fragment thereof. In embodiments, the cervical health biomarker is LGALS13 or a fragment thereof. In embodiments, the cervical health biomarker is LGALS14 or a fragment thereof. In embodiments, the cervical health biomarker is PAPPA or a fragment thereof. In embodiments, the cervical health biomarker is PGF or a fragment thereof. In embodiments, the cervical health biomarker is AFP or a fragment thereof. In embodiments, the cervical health biomarker is FLT or a fragment thereof. In embodiments, the cervical health biomarker is ENG or a fragment thereof. In embodiments, the cervical health biomarker is Spint1 or a fragment thereof. In embodiments, the cervical health biomarker is ADAM12 or a fragment thereof. In embodiments, the cervical health biomarker is MPO or a fragment thereof. In embodiments, the cervical health biomarker is CD68 or a fragment thereof. In embodiments, the cervical health biomarker is BMK13 or a fragment thereof. In embodiments, the cervical health biomarker is CD45 or a fragment thereof.

In embodiments, the cervical condition is a cervical infection, cervical bleeding, inflammation, or cervical cancer. In embodiments, the cervical condition is cervical infection. In embodiments, the cervical condition is bleeding. In embodiments, the cervical condition is inflammation. In embodiments, the cervical condition is cervical cancer.

In embodiments, the method further includes identifying a gynecological cancer. For example, identification of an elevated level or a decreased level of at least one cervical health biomarker relative to a standard control is indicative of a gynecological cancer. In embodiments, the gynecological cancer includes leiomyoma, leiomyosarcoma, uterine cancer, endometrial cancer, or ovarian cancer. In embodiments, the gynecological cancer is leiomyoma. In embodiments, the gynecological cancer is leiomyosarcoma. In embodiments, the gynecological cancer is uterine cancer. In embodiments, the gynecological cancer is endometrial cancer. In embodiments, the gynecological cancer is ovarian cancer. In embodiments, detecting an elevated level or decreased level of a cervical health biomarker relative to a standard control may be indicative of ovarian cancer. Thus, in embodiments, the cervical health biomarker is carbohydrate antigen 125 (CA125), epithelial cellular adhesion molecule (EpCAM), Carcinoembryonic antigen (CEA), Human epididymis protein 4 (HE4), Breast cancer type 1 susceptibility protein (BRCA1), transferrin, Osteopontin (OPN), Kallikreins (KLKs), or a combination or fragment thereof. In embodiments, an elevated level or decreased level of a cervical health biomarker relative to a standard control may be indicative of cervical cancer. Thus, in embodiments, the cervical health biomarker is a nucleic acid or fragment thereof derived from a human papilloma virus (HPV). In embodiments, the cervical health biomarker is viral protein E6 or viral protein E7 derived from HPV. In embodiments, the cervical health biomarker is CA125, CEA, cancer antigen 19-9 (CA 19-9), SCC antigen (SCCA), cytokeratin 19 fragment antigen 21-1 (CYFRA 21-1), C-reactive protein (hs-CRP) or a combination or fragment thereof. In embodiments, the gynecological cancer is uterine cancer or endometrial cancer. In embodiments, detecting an elevated level or decreased level of a cervical health biomarker relative to a standard control may be indicative of uterine cancer or endometrial cancer. Thus, in embodiments, the cervical health biomarker is CEA, HE4, or hs-CRP. In embodiments, the gynecological cancer is leiomyosarcoma. In embodiments, detecting an elevated level or decreased level of a cervical health biomarker relative to a standard control may be indicative of leiomyosarcoma. Thus, in embodiments, the cervical health biomarker is CA-125, LDH, TIMP-1, SMOC2, SATB2 or a combination or fragment thereof. In embodiments, the gynecological cancer is teratoma. In embodiments, detecting an elevated level or decreased level of a cervical health biomarker relative to a standard control may be indicative of teratoma. Thus, in embodiments, the cervical health biomarker is B-HCG or fragment thereof. In embodiments, the gynecological cancer is choriocarcinoma. In embodiments, detecting an elevated level or decreased level of a cervical health biomarker relative to a standard control may be indicative of choriocarcinoma. Thus, in embodiments, the cervical health biomarker is CA-125, SA19-9, AFP or a combination or fragment thereof.

In embodiments, an elevated level or decreased level of at least one biomarker relative to a standard control may be indicative of a cervical infection. In embodiments, the cervical infection is Clamydia trachomatis infection, HPV infection, HSV infection, HIV infection, herpes simplex virus 2 infection, Neisseria gonorrheae infection, Treponema pallidum (Syphilis) infection, candida infection, Trichomonas infection, or Gardnerella infection. In embodiments, the cervical infection may be Chlamydia trachomatis infection, Neisseria gonorreocae infection, or human papilloma virus (HPV) infection. In embodiments, the cervical infection is HPV infection. Thus, in embodiments, the biomarker may be anti-TroA IgG antibody, anti-HtrA IgG antibody, anti-MOMP IgG antibody or a combination or fragment thereof.

In embodiments, an elevated level or decreased level of one or more of IL1-alpha, IL1-beta, IL6, IL8, TNF, IFN-alpha, IFN-beta, IFN-gamma relative to a standard control, may be indicative of inflammation. Thus, in embodiments, the cervical health biomarker is IL1-alpha, IL1-beta, IL-6, IL-8, TNF, IFN-alpha, IFN-beta, or IFN-gamma or a combination or fragment thereof. In embodiments, the cervical health biomarker is IL1-alpha or a fragment thereof. In embodiments, the cervical health biomarker is IL1-beta or a fragment thereof. In embodiments, the cervical health biomarker is IL-6 or a fragment thereof. In embodiments, the cervical health biomarker is IL-8 or a fragment thereof. In embodiments, the cervical health biomarker TNF or a fragment thereof. In embodiments, the cervical health biomarker is IFN-alpha or a fragment thereof. In embodiments, the cervical health biomarker is IFN-beta or a fragment thereof. In embodiments, the cervical health biomarker is IFN-gamma or a fragment thereof.

In embodiments, the method further includes identifying a gynecological condition. In embodiments, identifying a gynecological condition includes detection an elevated level or decreased level of at least one biomarker relative to a standard control, wherein the increased or decreased level of the at least one biomarker is indicative of the gynecological condition. For example, in embodiments, the gynecological condition is leiomyoma or uterine fibroids, endometriosis, adenomyosis, polycystic ovarian syndrome, ovarian cyst, or hydatidiform mole. In embodiments, the gynecological condition is leiomyoma or uterine fibroids. Thus, in embodiments, the biomarker is PLP1, CA125, CA19-9 or a combination or fragment thereof. In embodiments, the gynecological condition is endometriosis. Thus, in embodiments, the biomarker is a combination of CA125, IL-8 and TNF-a. In embodiments, the biomarker is IL-6, MCP1, or INF-gamma or a fragment or combination thereof. In embodiments, the gynelogical condition is polycystic ovary syndrome. Thus, in embodiments, the biomarker is IL-1b, IL-6, TNF-alpha, CRP, MMP9, HGF, or a fragment or combination thereof. In embodiments, the gynecological condition is endometriosis. Thus, in embodiments, the biomarker is surviving (BIRC5), CA-125, IL-6, IL-8, TNF, or a fragment or combination thereof. In embodiments, the gynecological condition is adenomyosis. Thus, in embodiments, the biomarker is alpha-1 antitrypsin (A1AT), vitamin D-binding protein (VDBP), or a combination or fragment thereof.

In embodiments, biomarkers detected in cervical fluid samples may allow for more accurate and earlier identification of pregnancy-associated risks and conditions compared to identification of said risks and conditions by analysis of serum or blood-based biomarkers. For example, cervical fluid samples lack features which contribute to biases associated with detection of biomarkers in blood, for example, blood volume, gestational age, and placental size dependent secretion effects. Thus, identification of elevated or decreased levels of at least one biomarker (e.g. biomarkers derived from EVT cells) in a cervical fluid sample may allow for accurate detection of pregnancy-related disorders in early gestation. In embodiments, detection of an elevated or decreased level of a biomarker in a cervical fluid sample can be at least 10%, at least 15%, at least 20%, at least 25%, at least 30%, at least 40%, at least 50%, at least 60%, at least 70%, at least 80%, at least 90%, or at least 100% more accurate in the indication of early gestational complications than serum- or blood-based analysis.

For the method provided herein, in embodiments, the pregnancy-associated risk or condition is placental insufficiency, pregnancy-induced hypertension, placental abruption, pregnancy loss, miscarriage, preeclampsia, eclampsia, hemolysis, elevated liver enzymes and low platelet count (HELLP) syndrome, fetal growth restriction, intrauterine growth restriction, preterm birth, low birthweight, placenta percreta, placenta increta, placenta previa, gestational hypertension, gestational thrombosis, stillbirth, placental infarction, or a combination thereof. In embodiments, the pregnancy-associated risk or condition is placental insufficiency. In embodiments, the pregnancy-associated risk or condition is pregnancy-induced hypertension. In embodiments, the pregnancy-associated risk or condition is placental abruption. In embodiments, the pregnancy-associated risk or condition is pregnancy loss. In embodiments, the pregnancy-associated risk or condition is miscarriage. In embodiments, the pregnancy-associated risk or condition is preeclampsia. In embodiments, the pregnancy-associated risk or condition is eclampsia. In embodiments, the pregnancy-associated risk or condition is hemolysis. In embodiments, the pregnancy-associated risk or condition is HELLP (Hemolysis, Elevated Liver enzymes and Low Platelets) syndrome. In embodiments, the pregnancy-associated risk or condition is fetal growth restriction. In embodiments, the pregnancy-associated risk or condition is intrauterine growth restriction. In embodiments, the pregnancy-associated risk or condition is preterm birth. In embodiments, the pregnancy-associated risk or condition is low birthweight. In embodiments, the pregnancy-associated risk or condition is placenta percreta. In embodiments, the pregnancy-associated risk or condition is placenta increta. In embodiments, the pregnancy-associated risk or condition is placenta previa. In embodiments, the pregnancy-associated risk or condition is gestational hypertension. In embodiments, the pregnancy-associated risk or condition is gestational thrombosis. In embodiments, the pregnancy-associated risk or condition is stillbirth. In embodiments, the pregnancy-associated risk or condition is placental infarction.

In embodiments, the elevated level or decreased level of the biomarker can be indicative of an early gestational complication. As used herein, “early gestational complication” refers to a pregnancy-associated risk or condition that occurs prior to the 24th week of pregnancy as measured by gestational age (e.g. the number of weeks that have elapsed since the first day of the subject's last menstrual period). In embodiments, an early gestational complication refers to a pregnancy-associated risk or condition that occurs prior to the 10^{th} week of pregnancy as measured by gestational age. In embodiments, an early gestational complication refers to a pregnancy-associated risk or condition that occurs prior between the 4^{th} and 10^{th} week of pregnancy as measured by gestational age. In embodiments, an early gestational complication refers to a pregnancy-associated risk or condition that occurs prior between the 4^{th} and 7^{th} week of pregnancy as measured by gestational age. In embodiments, the early gestational complication includes gestational diabetes, miscarriage, ectopic pregnancy, placenta accreta, placental abruption, anembryonic pregnancy (e.g. blighted ovum), or molar pregnancy. In embodiments, placental dysfunction or insufficiency, pregnancy-induced hypertension, placental abruption, pregnancy loss, miscarriage, preeclampsia, eclampsia, Hemolysis Elevated Liver enzymes and Low Platelet (HELLP) syndrome, fetal growth restriction, intrauterine growth restriction, preterm birth, or low birthweight is denoted as an early gestational complication when detected prior to the 24th week of pregnancy.

Thus, in embodiments, the elevated level or decreased level of the biomarker is indicative of a pregnancy-associated risk or complication that occurs prior to the 24^{th} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 22^{nd} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 20^{th} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 18^{th} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 16^{th} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 14^{th} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 12^{th} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 10^{th} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 8^{th} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 6^{th} week of pregnancy as measured by the gestational age (e.g. the number of weeks that have elapsed since the first day of the subject's last menstrual period). In embodiments, the elevated level or decreased level of the biomarker is indicative of a pregnancy-associated risk or complication that occurs prior to the 5^{th} week of pregnancy as measured by the gestational age (e.g. the number of weeks that have elapsed since the first day of the subject's last menstrual period).

In embodiments, the elevated level of a biomarker is indicative of an early gestational complication. In embodiments, the decreased level of a biomarker is indicative of an early gestational complication. In embodiments, the pregnancy-associated risk or condition is an early gestational complication including malplacentation, placental dysfunction, placental insufficiency, pregnancy loss, preeclampsia, or fetal growth restriction. In embodiments, the early gestational complication is malplacentation. In embodiments, the early gestational complication is placental dysfunction. In embodiments, the early gestational complication is placental insufficiency. In embodiments, the early gestational complication is pregnancy loss. In embodiments, the early gestational complication preeclampsia. In embodiments, the early gestational complication is fetal growth restriction.

Malplacentation refers to the sub-optimal perfusion of the developing placenta due to reduced uterine artery blood flow. Malplacentation can contribute to several additional perinatal disorders, including early pregnancy loss (EPL), preterm birth (PTB), fetal growth restriction (FGR), and preeclampsia (PE).

Placental dysfunction refers to a pregnancy complication in which the placenta, which delivers oxygen and nutrients into the fetal bloodstream, fails to properly support a developing fetus. Placental dysfunction can contribute to fetal growth restriction.

Placental insufficiency refers to a condition whereby there is a failure of placental vascular remodeling, leading to a failure of placentation resulting in acidosis and fetal hypoxemia. Placental insufficiency can contribute to fetal growth restriction or fetal demise.

Preterm birth refers to the delivery of babies born alive before 37 weeks of pregnancy are completed.

Pregnancy loss, also referred to as miscarriage or spontaneous abortion, generally refers to a nonviable intrauterine pregnancy up to twenty weeks gestation. Early pregnancy loss, which occurs in the first trimester, is the most common type of pregnancy loss

Preeclampsia refers to a high blood pressure (hypertension) disorder that can occur during pregnancy. Severe preeclampsia is new onset hypertension in pregnancy after twenty weeks gestation with proteinuria. Proteinuria, also called albuminuria, is elevated protein in the urine. Proteinuria is not a disease in and of itself but a symptom of certain conditions affecting the kidneys.

Fetal growth restriction refers to a condition in which an unborn baby (fetus) is smaller than expected for the number of weeks of pregnancy (also known as gestational age). Between 6-16% of fetuses born growth restricted which survive to age two years score two or more standard deviations below average on developmental indices. Abnormal EVT function has been commonly found in FGR placentas.

### Methods Including Mass Spectrometry Techniques

In pregnancy, the placental extra-villous trophoblast (EVT) cell lineage establishes the maternal-fetal interface where nutrients and gas may exchange from maternal cells to the fetus. During pregnancy, EVT may accumulate in the cervix, allowing for detection of EVT biomarkers. EVTs can be obtained from the cervix using noninvasive methods, for example using a cytology brush. Aberrant expression levels of biomarkers on EVT can be a sign of abnormal placentation and can severely impair implantation and promotion of maternal blood flow to the implantation site. Abnormal EVT function has been commonly associated with fetal growth restriction (FGR), preeclamptic (PE) placentas, and other pregnancy-related risks and conditions. Currently, the only treatment for either FGR or PE is to deliver the baby, often prematurely. Applicant describes herein methods for accurately identifying pregnancy-associated risks or conditions early in the gestational period, including use of using mass spectrometry (MS) for analysis of biological samples (e.g. cervical fluid samples) including EVT cells, thereby allowing early intervention or treatment for pregnancy-associated risks or conditions. The methods further allow for non-invasive collection of biological samples (e.g. cervical fluid samples) from the cervix for identifying risks or conditions that adversely affect pregnancy. In embodiments, the methods enable detection of an elevated level or decreased level of at least one biomarker in a biological sample without isolating biomarker-expressing cells (e.g. EVT cells) from the biological sample. In embodiments, the methods enable detection of an elevated level or decreased level of at least one biomarker in a biological sample without enriching biomarker-expressing cells (e.g. EVT cells) in the biological sample. Thus, in an aspect is provided a method of identifying one or more pregnancy-associated risks or conditions in a subject, the method including: a) obtaining a biological sample from the cervix of the subject, wherein the biological sample includes extravillous trophoblast (EVT) cells; b) performing single-cell time-of-flight mass spectrometry (CyTOF-MS) on the biological sample to generate an output; and c) determining an elevated level or a decreased level of at least one biomarker in the biological sample relative to a standard control based on the output, thereby identifying the one or more pregnancy-associated risks or conditions.

As described above, Applicant has discovered a method for sensitive and accurate detection of an elevated level or decreased level of a biomarker in a biological sample including EVT. The method provided herein allows for determination of an elevated level or decreased level of the biomarker expressed by an EVT cell without isolation, purification, or enrichment of the EVT cell from the biological sample (e.g. cervical fluid samples). Thus, embodiments, the method does not include isolation, purification, or enrichment of EVT cells from the biological sample. For example, an elevated level or decreased level of a biomarker (e.g. a biomarker expressed by an EVT cell) may be detected in the biological sample without isolating the EVT cells. Thus, in embodiments, the method does not include isolating the EVT. For example, in embodiments, the method does not include isolating the EVT cells from components naturally present in the biological sample. Thus, in embodiments, labeling an EVT cell (e.g. with an antibody) for CyTOF-MS methods does not denote isolating the EVT cell. In embodiments, contacting the EVT cells with an antibody capable of binding a biomarker expressed by the EVT cell does not denote isolation of the EVT cell. For example, in embodiments, contacting the EVT cell with a detectable moiety (e.g. a labeled antibody, a detectable-moiety conjugated antibody) does not denote isolation of the EVT cell.

In embodiments, the method provided herein allows for sensitive and accurate detection of an elevated level or decreased level of a biomarker in a biological sample (e.g. cervical fluid samples) without enrichment of EVT. “Enrichment” as used herein refers to separating non-EVT components from the biological sample including the EVT cells and biological material. For example, in embodiments, enrichment of EVT cells includes increasing the concentration of EVT cells in a biological sample. In embodiments, enrichment of EVT cells includes increasing the number of EVT cells per unit volume. Thus, in embodiments, the method does not include enrichment of the EVT cells from a biological sample including the EVT cells and biological material. Thus, in embodiments, the step of labeling an EVT cell (e.g. with an antibody) for CyTOF-MS methods does not denote enriching the EVT cell. In embodiments, contacting the EVT cells with an antibody capable of binding a biomarker expressed by the EVT cell does not denote enrichment of the EVT cell. For example, in embodiments, contacting the EVT cell with a detectable moiety (e.g. a labeled antibody, a detectable-moiety conjugated antibody) does not denote enrichment of the EVT cell in the biological sample.

As described throughout the specification, for the method provided herein, in embodiments the elevated level or decreased level of at least one biomarker is measured by mass spectrometry. Mass Spectrometry (MS) refers to an analytical tool useful for measuring the mass-to-charge ratio (m/z) of one or more molecules present in a sample. Mass cytometry (also known as time-of-flight mass cytometry, cytometry by time-of-flight, CyTOF, or CyTOF-MS) refers to a mass spectrometry technique based on inductively coupled plasma mass spectrometry and time of flight mass spectrometry used for the determination of the properties of cells. In mass cytometry, antibodies may be conjugated with a detectable moiety (e.g. isotopically pure element) to label cellular proteins, thereby allowing detection of biomarkers (e.g. proteins). For example, the biological sample may be contacted with one or more antibodies specific for a protein biomarker. The protein may be expressed on the surface of a cell (e.g. EVT cell) or expressed within in the cell. Binding of the antibody to the protein thereby allows detection of the protein. For the methods provided herein, in embodiments, cells (e.g. EVT cells) are nebulized and sent through an argon plasma, which ionizes the metal-conjugated antibodies. The metal signals are then analyzed by a time-of-flight spectrometer. CyTOF-MS can then provide an output, wherein the output may include cell phenotype, transcription level of one or more gene, or expression level of one or more protein. In embodiments, the output includes detection of one or more biomarkers expressed by an EVT cell. In embodiments, a plurality of outputs may be obtained, thereby allowing multiplexing of biomarkers. For the methods provided herein, in embodiments, mass spectrometry may be used in combination with one or more additional methods (e.g. flow cytometry) for identifying the at least one biomarker provided herein.

In embodiments, multiple outputs are generated thereby allowing multiplex detection of at least 1, 2, 3, 4, 5, 6, 7, 8, 9, 10, 15, 20, 30 40, 50, 60, 70, 80, 90, or 100 biomarkers. In embodiments, at least about 1, at least about 5, at least about 10, at least about 15, at least about 20, at least about 30, at least about 40, at least about 50, at least about 60, at least about 70, at least about 80, at least about 90, at least about 100, or more biomarkers can be identified in the cervical fluid sample.

In embodiments, the method further includes performing flow cytometry on the biological sample. Flow cytometry refers to a technique used to detect and measure physical and chemical characteristics of a population of cells or particles. In flow cytometry, a sample is suspended in a fluid and injected into a flow cytometer. The sample is focused to flow one cell at a time through a laser beam, where the light scattered is characteristic to the cells and their components. Flow cytometry can be combined with fluorescence labeling to further differentiate between cells. Flow cytometry can be used for cell counting, cell sorting, determination of cell characteristic, detection of microorganisms, and detection of biomarkers. In embodiments, flow cytometry includes contacting the biological sample with one or more antibodies specific for a biomarker as provided herein including embodiments thereof. In embodiments, the antibody is attached to a detectable moiety, thereby allowing detection of the biomarker.

Using CyTOF and/or flow cytometry, at least about 1, at least about 5, at least about 10, at least about 15, at least about 20, at least about 30, at least about 40, at least about 50, at least about 60, at least about 70, at least about 80, at least about 90, at least about 100, or more cellular biomarkers can be identified in each individual cell simultaneously.

In embodiments, CyTOF-MS and/or flow cytometry is performed on the biological sample after obtaining said sample and without subsequent processing steps. For example, in embodiments, the method does not include addition of preservatives, or fixatives after obtaining the biological sample. Alternatively, in embodiments, the biological sample includes additional processing prior to performing CyTOF-MS and/or flow cytometry. For example, in embodiments, the biological sample may be washed. In embodiments, the biological sample may be filtered. In embodiments, the method further includes counting the number of cells in the biological sample. For example, cell counting may be performed through manual counting methods, automated cell counters, or flow cytometry-based methods. In embodiments, the method further includes determining viability of the cells. for example, determining cell viability may include use of a cell membrane integrity dye or enzymatic assay. In embodiments, the method includes addition of a preservative or fixative to the biological sample. In embodiments, the preservative or fixative includes paraformaldehyde (PFA), glutaraldehyde, isopropanol, Methanol, ethanol, formaldehyde, Maxpar® Fix and Perm Buffer, PROT1, VCM media, or cell transport media. In embodiments, the method includes permeabilization of the cells. For example, in embodiments, the method includes adding a permeabilization agent to the biological sample. In embodiments, permeabilization of the cells allows delivery of antibodies (e.g. detectable-moiety conjugated antibodies) into the cell. In embodiments, the permeabilization agent includes methanol, ethanol, saponin-based buffers, or any other permeabilizing agent known at the time of filing. In embodiments, the method includes storing the sample prior to performing CyTOF-MS and/or flow cytometry. For example, in embodiments, the biological sample is frozen (e.g. at −20 C) and subsequently thawed prior to performing CyTOF-MS and/or flow cytometry.

In embodiments, the method is completed within 24 hours. In embodiments, CyTOF-MS and/or flow cytometry is performed on the biological sample within 24 hours of obtaining the biological sample from the subject. In embodiments, CyTOF-MS and/or flow cytometry is performed within 2 hours, within 3 hours, within 4 hours, within 5 hours, within 6 hours, within 7 hours, within 8 hours, within 9 hours, within 10 hours, within 11 hours, within 12 hours, within 13 hours, within 14 hours, within 15 hours, within 16 hours, within 17 hours, within 18 hours, within 19 hours, within 20 hours, within 21 hours, within 22 hours, within 23 hours, or within 24 hours of obtaining the biological sample from the object.

For the methods provided herein, in embodiments, the biological sample is collected through a pap smear or other procedure that allows for the collection of biological sample from the cervix (e.g., a diva cup). In embodiments, the biological sample is obtained from a swab that has contacted a subject. In embodiments, a swab can obtain a biological sample by contacting a urethral canal, a vaginal canal, or an anal canal. In embodiments, a biological sample taken from the vaginal canal (e.g., using a cytobrush) can comprise a biological sample taken from the endo or exo cervix, which is positioned at one end of the vaginal canal. In embodiments, the biological sample further includes biological material derived from the cervix of the sample. In embodiments, the biological sample further includes biological material derived from the subject (e.g., maternal cells). In embodiments, the biological sample further includes biological material derived from the fetus/placenta (e.g., fetal cells or placental cells).

As described herein, in embodiments, the biological sample (e.g. cervical fluid sample) includes EVT cells and biological material. In embodiments, “biological material” refers to any non-EVT component of the biological sample. In embodiments, biological material may refer to any non-EVT component of the biological sample including bodily fluids (e.g. mucous, blood, serum, plasma, platelets, red blood cells, or tissue) present in the biological sample. In embodiments, biological material refers to any biomolecule within the biological sample that is not bound to, attached to or associated with an EVT cell. In embodiments, biological material refers to any biomolecule that is not expressed by an EVT cell. In embodiments, biological material refers to any non-EVT cell. For example, in embodiments, the biological material includes mucous, a maternal cell, biological fluid, or a combination thereof. In embodiments, the biological material includes mucous. In embodiments, the biological material includes a maternal cell. In embodiments, the biological material includes biological fluid.

For the methods provided herein, in embodiments, the biological sample (e.g. cervical fluid sample) includes components (e.g. biological material (e.g. mucous, cells, blood, DNA, protein, etc.)). that have transferred to the cervix from elsewhere in the subject's body. For example, in embodiments, components of the biological sample (e.g. biological material) originates from the uterus and/or the placenta of the subject. For example, the biological sample may include components (e.g. biological material) derived from the uterus and/or placenta of the subject which have accumulated in the cervix. For example, the biological sample may include mucous, maternal cells, a biological fluid, or any combination thereof which have accumulated in the cervix from elsewhere in the subject's body.

In embodiments, the biological material includes at least about 70% weight by volume (w/v) of the biological sample (e.g. cervical fluid sample). In embodiments, the biological material includes at least about 75% weight by volume (w/v) of the biological sample. In embodiments, the biological material includes at least about 80% weight by volume (w/v) of the biological sample. In embodiments, the biological material includes at least about 85% weight by volume (w/v) of the biological sample. In embodiments, the biological material includes at least about 90% weight by volume (w/v) of the biological sample. In embodiments, the biological material includes at least about 91% weight by volume (w/v) of the biological sample. In embodiments, the biological material includes at least about 92% weight by volume (w/v) of the biological sample. In embodiments, the biological material includes at least about 93% weight by volume (w/v) of the biological sample. In embodiments, the biological material includes at least about 94% weight by volume (w/v) of the biological sample. In embodiments, the biological material includes at least about 95% weight by volume (w/v) of the biological sample. In embodiments, the biological material includes at least about 96% weight by volume (w/v) of the biological sample. In embodiments, the biological material includes at least about 97% weight by volume (w/v) of the biological sample. In embodiments, the biological material includes at least about 98% weight by volume (w/v) of the biological sample. In embodiments, the biological material includes at least about 99% weight by volume (w/v) of the biological sample.

In embodiments, the biological sample (e.g. cervical fluid sample) includes at least about 70% biological material as measured by mass percentage of solute in solution (w/w). In embodiments, the biological sample includes at least about 75% biological material as measured by mass percentage of solute in solution (w/w). In embodiments, the biological sample includes at least about 80% biological material as measured by mass percentage of solute in solution (w/w). In embodiments, the biological sample includes at least about 85% biological material as measured by mass percentage of solute in solution (w/w). In embodiments, the biological sample includes at least about 90% biological material as measured by mass percentage of solute in solution (w/w). In embodiments, the biological sample includes at least about 91% biological material as measured by mass percentage of solute in solution (w/w). In embodiments, the biological sample includes at least about 92% biological material as measured by mass percentage of solute in solution (w/w). In embodiments, the biological sample includes at least about 93% biological material as measured by mass percentage of solute in solution (w/w). In embodiments, the biological sample includes at least about 94% biological material as measured by mass percentage of solute in solution (w/w). In embodiments, the biological sample includes at least about 95% biological material as measured by mass percentage of solute in solution (w/w). In embodiments, the biological sample includes at least about 96% biological material as measured by mass percentage of solute in solution (w/w). In embodiments, the biological sample includes at least about 97% biological material as measured by mass percentage of solute in solution (w/w). In embodiments, the biological sample includes at least about 98% biological material as measured by mass percentage of solute in solution (w/w). In embodiments, the biological sample includes at least about 99% biological material as measured by mass percentage of solute in solution (w/w).

In embodiments, the biological sample (e.g. cervical fluid sample) includes at least about 70% biological material as measured by volume by volume percentage (v/v). In embodiments, the biological sample includes at least about 80% biological material as measured by volume by volume percentage (v/v). In embodiments, the biological sample includes at least about 85% biological material as measured by volume by volume percentage (v/v). In embodiments, the biological sample includes at least about 90% biological material as measured by volume by volume percentage (v/v). In embodiments, the biological sample includes at least about 91% biological material as measured by volume by volume percentage (v/v). In embodiments, the biological sample includes at least about 92% biological material as measured by volume by volume percentage (v/v). In embodiments, the biological sample includes at least about 93% biological material as measured by volume by volume percentage (v/v). In embodiments, the biological sample includes at least about 94% biological material as measured by volume by volume percentage (v/v). In embodiments, the biological sample includes at least about 95% biological material as measured by volume by volume percentage (v/v). In embodiments, the biological sample includes at least about 96% biological material as measured by volume by volume percentage (v/v). In embodiments, the biological sample includes at least about 97% biological material as measured by volume by volume percentage (v/v). In embodiments, the biological sample includes at least about 98% biological material as measured by volume by volume percentage (v/v). In embodiments, the biological sample includes at least about 99% biological material as measured by volume by volume percentage (v/v).

In embodiments, the biological sample (e.g. cervical fluid sample) includes at least about at least about 70%, at least about 80%, at least about 82%, at least about 84%, at least about 86%, at least about 88%, at least about 90%, at least about 91%, at least about 92%, at least about 93%, at least about 94%, at least about 95%, at least about 96%, at least about 97%, at least about 98%, or at least about 99% biological materials other than EVT cells, as measured by mass percentage of solute in solution (w/w).

In embodiments, the biological sample includes at least about at most about 99%, at most about 98%, at most about 97%, at most about 96%, at most about 95%, at most about 94%, at most about 93%, at most about 92%, at most about 91%, at most about 90%, at most about 88%, at most about 86%, at most about 84%, at most about 82%, at most about 80%, or at most about 70% biological materials other than EVT cells, as measured by mass percentage of solute in solution (w/w).

In embodiments, the biological sample includes at least about at least about 70%, at least about 80%, at least about 82%, at least about 84%, at least about 86%, at least about 88%, at least about 90%, at least about 91%, at least about 92%, at least about 93%, at least about 94%, at least about 95%, at least about 96%, at least about 97%, at least about 98%, or at least about 99% biological materials other than EVT cells, as measured by volume by volume percentage (v/v).

In embodiments, the biological sample includes at least about at most about 99%, at most about 98%, at most about 97%, at most about 96%, at most about 95%, at most about 94%, at most about 93%, at most about 92%, at most about 91%, at most about 90%, at most about 88%, at most about 86%, at most about 84%, at most about 82%, at most about 80%, or at most about 70% biological materials other than EVT cells, as measured by volume by volume percentage (v/v).

In embodiments, the biological sample includes at least about at least about 70%, at least about 80%, at least about 82%, at least about 84%, at least about 86%, at least about 88%, at least about 90%, at least about 91%, at least about 92%, at least about 93%, at least about 94%, at least about 95%, at least about 96%, at least about 97%, at least about 98%, or at least about 99% biological materials other than EVT cells, as measured by weight by volume percentage (w/v).

In embodiments, the biological sample includes at least about at most about 99%, at most about 98%, at most about 97%, at most about 96%, at most about 95%, at most about 94%, at most about 93%, at most about 92%, at most about 91%, at most about 90%, at most about 88%, at most about 86%, at most about 84%, at most about 82%, at most about 80%, or at most about 70% biological materials other than EVT cells, as measured by weight by volume percentage (w/v).

In embodiments, the method includes obtaining from about 100 uL to about 2 mL biological sample. In embodiments, the method includes obtaining from about 200 uL to about 2 mL biological sample. In embodiments, the method includes obtaining from about 300 uL to about 2 mL biological sample. In embodiments, the method includes obtaining from about 400 uL to about 2 mL biological sample. In embodiments, the method includes obtaining from about 500 uL to about 2 mL biological sample. In embodiments, the method includes obtaining from about 600 uL to about 2 mL biological sample. In embodiments, the method includes obtaining from about 700 uL to about 2 mL biological sample. In embodiments, the method includes obtaining from about 800 uL to about 2 mL biological sample. In embodiments, the method includes obtaining from about 900 uL to about 2 mL biological sample. In embodiments, the method includes obtaining from about 1 mL to about 2 mL biological sample. In embodiments, the method includes obtaining from about 1.1 mL to about 2 mL biological sample. In embodiments, the method includes obtaining from about 1.2 mL to about 2 mL biological sample. In embodiments, the method includes obtaining from about 1.3 mL to about 2 mL biological sample. In embodiments, the method includes obtaining from about 1.4 mL to about 2 mL biological sample. In embodiments, the method includes obtaining from about 1.5 mL to about 2 mL biological sample. In embodiments, the method includes obtaining from about 1.6 mL to about 2 mL biological sample. In embodiments, the method includes obtaining from about 1.7 mL to about 2 mL biological sample. In embodiments, the method includes obtaining from about 1.8 mL to about 2 mL biological sample. In embodiments, the method includes obtaining from about 1.9 mL to about 2 mL biological sample.

In embodiments, the method includes obtaining from about 100 uL to about 1.9 mL biological sample. In embodiments, the method includes obtaining from about 100 uL to about 1.8 mL biological sample. In embodiments, the method includes obtaining from about 100 uL to about 1.7 mL biological sample. In embodiments, the method includes obtaining from about 100 uL to about 1.6 mL biological sample. In embodiments, the method includes obtaining from about 100 uL to about 1.5 mL biological sample. In embodiments, the method includes obtaining from about 100 uL to about 1.4 mL biological sample. In embodiments, the method includes obtaining from about 100 uL to about 1.3 mL biological sample. In embodiments, the method includes obtaining from about 100 uL to about 1.2 mL biological sample. In embodiments, the method includes obtaining from about 100 uL to about 1.1 mL biological sample. In embodiments, the method includes obtaining from about 100 uL to about 1 mL biological sample. In embodiments, the method includes obtaining from about 100 uL to about 900 uL biological sample. In embodiments, the method includes obtaining from about 100 uL to about 800 uL biological sample. In embodiments, the method includes obtaining from about 100 uL to about 700 uL biological sample. In embodiments, the method includes obtaining from about 100 uL to about 600 uL biological sample. In embodiments, the method includes obtaining from about 100 uL to about 500 uL biological sample. In embodiments, the method includes obtaining from about 100 uL to about 400 uL biological sample. In embodiments, the method includes obtaining from about 100 uL to about 300 uL biological sample. In embodiments, the method includes obtaining from about 100 uL to about 200 uL biological sample. In embodiments, the method includes obtaining from 100 uL, 200 uL, 300 uL, 400 uL, 500 uL, 600 700 uL, 800 uL, 900 uL, 1 mL, 1.1 mL, 1.2 mL, 1.3 mL, 1.4 mL, 1.5 mL, 1.6 mL, 1.7 mL, 1.8 mL, 1.9 mL, or 2 mL biological sample.

For the method provided herein, in embodiments, step a) may be performed a plurality of times. For example, wherein the method includes multiplex detection of an elevated or a decreased level of a plurality of biomarkers, step a) may be performed a plurality of times. For example, step a) may be performed at least once, twice, three times, four times, five times, six times, seven times, eight times, nine times or ten times. In embodiments, step a) is performed at least once. In embodiments, step a) is performed at least two times. In embodiments, step a) is performed at least 3 times. In embodiments, step a) is performed at least 4 times. In embodiments, step a) is performed at least 5 times. In embodiments, step a) is performed at least 6 times. In embodiments, step a) is performed at least 7 times. In embodiments, step a) is performed at least 8 times. In embodiments, step a) is performed at least 9 times. In embodiments, step a) is performed at least 10 times. In embodiments, step a) is performed at least 15 times. In embodiments, step a) is performed at least 20 times. In embodiments, step a) is performed once for each level of biomarker identified.

In embodiments, the biological sample includes at least about 25 cells per 200 uL volume. In embodiments, the biological sample includes at least about 25 cells per 200 uL volume. In embodiments, the biological sample includes at least about 25 cells per 300 uL volume. In embodiments, the biological sample includes at least about 25 cells per 400 uL volume. In embodiments, the biological sample includes at least about 25 cells per 500 uL volume. In embodiments, the biological sample includes at least about 25 cells per 600 uL volume. In embodiments, the biological sample includes at least about 25 cells per 700 uL volume. In embodiments, the biological sample includes at least about 25 cells per 800 uL volume. In embodiments, the biological sample includes at least about 25 cells per 900 uL volume. In embodiments, the biological sample includes at least about 25 cells per 1 mL volume. In embodiments, the biological sample includes at least about 25 cells per 1.1 mL volume. In embodiments, the biological sample includes at least about 25 cells per 1.2 mL volume. In embodiments, the biological sample includes at least about 25 cells per 1.3 mL volume. In embodiments, the biological sample includes at least about 25 cells per 1.4 mL volume. In embodiments, the biological sample includes at least about 25 cells per 1.5 mL volume. In embodiments, the biological sample includes at least about 25 cells per 1.6 mL volume. In embodiments, the biological sample includes at least about 25 cells per 1.6 mL volume. In embodiments, the biological sample includes at least about 25 cells per 1.7 mL volume. In embodiments, the biological sample includes at least about 25 cells per 1.8 mL volume. In embodiments, the biological sample includes at least about 25 cells per 1.9 mL volume. In embodiments, the biological sample includes at least about 25 cells per 2 mL volume. In embodiments, the cells include EVT cells and non-EVT cells (e.g. maternal cells, fetal cells, non-EVT placenta-specific cells, etc.). In embodiments, the cells include EVT cells and no other types of cells.

In embodiments, the biological sample includes at least about 50 cells per 200 uL to about 2 mL of volume. In embodiments, the biological sample includes at least about 50 cells per 200 uL volume. In embodiments, the biological sample includes at least about 50 cells per 300 uL volume. In embodiments, the biological sample includes at least about 50 cells per 400 uL volume. In embodiments, the biological sample includes at least about 50 cells per 500 uL volume. In embodiments, the biological sample includes at least about 50 cells per 600 uL volume. In embodiments, the biological sample includes at least about 50 cells per 700 uL volume. In embodiments, the biological sample includes at least about 50 cells per 800 uL volume. In embodiments, the biological sample includes at least about 50 cells per 900 uL volume. In embodiments, the biological sample includes at least about 50 cells per 1 mL volume. In embodiments, the biological sample includes at least about 50 cells per 1.1 mL volume. In embodiments, the biological sample includes at least about 50 cells per 1.2 mL volume. In embodiments, the biological sample includes at least about 50 cells per 1.3 mL volume. In embodiments, the biological sample includes at least about 50 cells per 1.4 mL volume. In embodiments, the biological sample includes at least about 50 cells per 1.5 mL volume. In embodiments, the biological sample includes at least about 50 cells per 1.6 mL volume. In embodiments, the biological sample includes at least about 50 cells per 1.6 mL volume. In embodiments, the biological sample includes at least about 50 cells per 1.7 mL volume. In embodiments, the biological sample includes at least about 50 cells per 1.8 mL volume. In embodiments, the biological sample includes at least about 50 cells per 1.9 mL volume. In embodiments, the biological sample includes at least about 50 cells per 2 mL volume. In embodiments, the cells include EVT cells and non-EVT cells (e.g. maternal cells, fetal cells, non-EVT placenta-specific cells, etc.). In embodiments, the cells include EVT cells and no other types of cells.

In embodiments, the biological sample includes at least about 100 cells per 200 uL to about 2 mL of volume. In embodiments, the biological sample includes at least about 100 cells per 200 uL volume. In embodiments, the biological sample includes at least about 100 cells per 300 uL volume. In embodiments, the biological sample includes at least about 100 cells per 400 uL volume. In embodiments, the biological sample includes at least about 100 cells per 500 uL volume. In embodiments, the biological sample includes at least about 100 cells per 600 uL volume. In embodiments, the biological sample includes at least about 100 cells per 700 uL volume. In embodiments, the biological sample includes at least about 100 cells per 800 uL volume. In embodiments, the biological sample includes at least about 100 cells per 900 uL volume. In embodiments, the biological sample includes at least about 100 cells per 1 mL volume. In embodiments, the biological sample includes at least about 100 cells per 1.1 mL volume. In embodiments, the biological sample includes at least about 100 cells per 1.2 mL volume. In embodiments, the biological sample includes at least about 100 cells per 1.3 mL volume. In embodiments, the biological sample includes at least about 100 cells per 1.4 mL volume. In embodiments, the biological sample includes at least about 100 cells per 1.5 mL volume. In embodiments, the biological sample includes at least about 100 cells per 1.6 mL volume. In embodiments, the biological sample includes at least about 100 cells per 1.6 mL volume. In embodiments, the biological sample includes at least about 100 cells per 1.7 mL volume. In embodiments, the biological sample includes at least about 100 cells per 1.8 mL volume. In embodiments, the biological sample includes at least about 100 cells per 1.9 mL volume. In embodiments, the biological sample includes at least about 100 cells per 2 mL volume. In embodiments, the cells include EVT cells and non-EVT cells (e.g. maternal cells, fetal cells, non-EVT placenta-specific cells, etc.). In embodiments, the cells include EVT cells and no other types of cells.

In embodiments, the biological sample includes from about 5 cells to about 100 cells per 2 mL volume. In embodiments, the biological sample includes from about 10 cells to about 100 cells per 2 mL volume. In embodiments, the biological sample includes from about 15 cells to about 100 cells per 2 mL volume. In embodiments, the biological sample includes from about 20 cells to about 100 cells per 2 mL volume. In embodiments, the biological sample includes from about 25 cells to about 100 cells per 2 mL volume. In embodiments, the biological sample includes from about 30 cells to about 100 cells per 2 mL volume. In embodiments, the biological sample includes from about 35 cells to about 100 cells per 2 mL volume. In embodiments, the biological sample includes from about 40 cells to about 100 cells per 2 mL volume. In embodiments, the biological sample includes from about 45 cells to about 100 cells per 2 mL volume. In embodiments, the biological sample includes from about 50 cells to about 100 cells per 2 mL volume. In embodiments, the biological sample includes from about 55 cells to about 100 cells per 2 mL volume. In embodiments, the biological sample includes from about 60 cells to about 100 cells per 2 mL volume. In embodiments, the biological sample includes from about 65 cells to about 100 cells per 2 mL volume. In embodiments, the biological sample includes from about 70 cells to about 100 cells per 2 mL volume. In embodiments, the biological sample includes from about 75 cells to about 100 cells per 2 mL volume. In embodiments, the biological sample includes from about 80 cells to about 100 cells per 2 mL volume. In embodiments, the biological sample includes from about 85 cells to about 100 cells per 2 mL volume. In embodiments, the biological sample includes from about 90 cells to about 100 cells per 2 mL volume. In embodiments, the biological sample includes from about 95 cells to about 100 cells per 2 mL volume. In embodiments, the cells include EVT cells and non-EVT cells (e.g. maternal cells, fetal cells, non-EVT placenta-specific cells, etc.). In embodiments, the cells include EVT cells and no other types of cells.

In embodiments, the biological sample includes from about 5 cells to about 95 cells per 2 mL volume. In embodiments, the biological sample includes from about 5 cells to about 90 cells per 2 mL volume. In embodiments, the biological sample includes from about 5 cells to about 85 cells per 2 mL volume. In embodiments, the biological sample includes from about 5 cells to about 80 cells per 2 mL volume. In embodiments, the biological sample includes from about 5 cells to about 75 cells per 2 mL volume. In embodiments, the biological sample includes from about 5 cells to about 70 cells per 2 mL volume. In embodiments, the biological sample includes from about 5 cells to about 65 cells per 2 mL volume. In embodiments, the biological sample includes from about 5 cells to about 60 cells per 2 mL volume. In embodiments, the biological sample includes from about 5 cells to about 55 cells per 2 mL volume. In embodiments, the biological sample includes from about 5 cells to about 50 cells per 2 mL volume. In embodiments, the biological sample includes from about 5 cells to about 45 cells per 2 mL volume. In embodiments, the biological sample includes from about 5 cells to about 40 cells per 2 mL volume. In embodiments, the biological sample includes from about 5 cells to about 35 cells per 2 mL volume. In embodiments, the biological sample includes from about 5 cells to about 30 cells per 2 mL volume. In embodiments, the biological sample includes from about 5 cells to about 25 cells per 2 mL volume. In embodiments, the biological sample includes from about 5 cells to about 20 cells per 2 mL volume. In embodiments, the biological sample includes from about 5 cells to about 15 cells per 2 mL volume. In embodiments, the biological sample includes from about 5 cells to about 10 cells per 2 mL volume. In embodiments, the cells include EVT cells and non-EVT cells (e.g. maternal cells, fetal cells, non-EVT placenta-specific cells, etc.). In embodiments, the cells include EVT cells and no other types of cells.

In embodiments, the biological sample includes no more than about 50 cells per 1 mL volume. In embodiments, the biological sample includes no more than about 45 cells per 1 mL volume. In embodiments, the biological sample includes no more than about 40 cells per 1 mL volume. In embodiments, the biological sample includes no more than about 35 cells per 1 mL volume. In embodiments, the biological sample includes no more than about 30 cells per 1 mL volume. In embodiments, the biological sample includes no more than about 25 cells per 1 mL volume. In embodiments, the biological sample includes no more than about 20 cells per 1 mL volume. In embodiments, the biological sample includes no more than about 15 cells per 1 mL volume. In embodiments, the biological sample includes no more than about 10 cells per 1 mL volume. In embodiments, the biological sample includes no more than about 5 cells per 1 mL volume. In embodiments, the biological sample includes no more than about 4 cells per 1 mL volume. In embodiments, the biological sample includes no more than about 3 cells per 1 mL volume. In embodiments, the biological sample includes no more than about 2 cells per 1 mL volume. In embodiments, the biological sample includes no more than about 1 cell per 1 mL volume.

In embodiments, the biological sample includes no more than about 10 cells per 0.5 milliliters (mL), no more than about 15 cells per 0.5 milliliters (mL), no more than about 20 cells per 0.5 milliliters (mL), no more than about 25 cells per 0.5 milliliters (mL), no more than about 30 cells per 0.5 milliliters (mL), no more than about 35 cells per 0.5 milliliters (mL), no more than about 40 cells per 0.5 milliliters (mL), no more than about 45 cells per 0.5 milliliters (mL), no more than about 50 cells per 0.5 milliliters (mL), no more than about 60 cells per 0.5 milliliters (mL), no more than about 70 cells per 0.5 milliliters (mL), no more than about 80 cells per 0.5 milliliters (mL), no more than about 90 cells per 0.5 milliliters (mL), no more than about 100 cells per 0.5 milliliters (mL), no more than about 200 cells per 0.5 milliliters (mL), no more than about 300 cells per 0.5 milliliters (mL), no more than about 400 cells per 0.5 milliliters (mL), no more than about 500 cells per 0.5 milliliters (mL), no more than about 1,000 cells per 0.5 milliliters (mL), no more than about 5,000 cells per 0.5 milliliters (mL), no more than about 10,000 cells per 0.5 milliliters (mL), or no more than about 100,000 cells per 0.5 milliliters (mL). In embodiments, the cells include EVT cells and non-EVT cells (e.g. maternal cells, fetal cells, non-EVT placenta-specific cells, etc.). In embodiments, the cells include EVT cells and no other types of cells.

In embodiments, the biological sample includes at least about 100,000 cells per 0.5 milliliters (mL), at least about 10,000 cells per 0.5 milliliters (mL), at least about 5,000 cells per 0.5 milliliters (mL), at least about 1,000 cells per 0.5 milliliters (mL), at least about 500 cells per 0.5 milliliters (mL), at least about 400 cells per 0.5 milliliters (mL), at least about 300 cells per 0.5 milliliters (mL), at least about 200 cells per 0.5 milliliters (mL), at least about 100 cells per 0.5 milliliters (mL), at least about 90 cells per 0.5 milliliters (mL), at least about 80 cells per 0.5 milliliters (mL), at least about 70 cells per 0.5 milliliters (mL), at least about 60 cells per 0.5 milliliters (mL), at least about 50 cells per 0.5 milliliters (mL), at least about 45 cells per 0.5 milliliters (mL), at least about 40 cells per 0.5 milliliters (mL), at least about 35 cells per 0.5 milliliters (mL), at least about 30 cells per 0.5 milliliters (mL), at least about 25 cells per 0.5 milliliters (mL), at least about 20 cells per 0.5 milliliters (mL), at least about 15 cells per 0.5 milliliters (mL), or at least about 10 cells per 0.5 milliliters (mL). In embodiments, the cells include EVT cells and non-EVT cells (e.g. maternal cells, fetal cells, non-EVT placenta-specific cells, etc.). In embodiments, the cells include EVT cells and no other types of cells.

In embodiments, the biological sample includes cervical cells. As described herein, cervical cells include cells originating from cervical tissue. For example, cervical cells originating from cervical tissue include cells lining the surface of the cervix. Cervical cells originating from cervical tissue include glandular cells, which have a column-shaped appearance, and squamous cells, which are thin and flat. Alternatively, the biological sample may include cells that have accumulated in the cervix (e.g., EVT cells or other placental cells). Thus, in embodiments, the EVT include EVT residing in or passing through the cervix. In embodiments, a cervical sample includes other body fluids or biological materials such as mucous, blood, or fetal DNA.

In embodiments, the biological sample includes placental cells. Placental cells are a type of cell derived from the extra-embryonic tissues that creates the placenta of the fetus' or newborn's placental blood or tissue. Placentas are composed of three layers of components with different cell types in each. The first layer comprises trophoblast cells, which are formed during the first stage of pregnancy and are the first cells to differentiate from the fertilized egg. The second layer comprises mesenchymal cells, mesenchymal derived macrophages, and fibroblasts. The third layer comprises fetal vascular cells, perivascular cells, and endothelial cells.

In embodiments, the biological sample includes less than 0.1% weight by volume (w/v) EVT. In embodiments, the biological sample includes less than 0.09% weight by volume (w/v) EVT. In embodiments, the biological sample includes less than 0.08% weight by volume (w/v) EVT. In embodiments, the biological sample includes less than 0.07% weight by volume (w/v) EVT. In embodiments, the biological sample includes less than 0.06% weight by volume (w/v) EVT. In embodiments, the biological sample includes less than 0.05% weight by volume (w/v) EVT. In embodiments, the biological sample includes less than 0.04% weight by volume (w/v) EVT. In embodiments, the biological sample includes less than 0.03% weight by volume (w/v) EVT. In embodiments, the biological sample includes less than 0.02% weight by volume (w/v) EVT. In embodiments, the biological sample includes less than 0.01% weight by volume (w/v) EVT. In embodiments, the biological sample includes less than 0.009% weight by volume (w/v) EVT. In embodiments, the biological sample includes less than 0.008% weight by volume (w/v) EVT. In embodiments, the biological sample includes less than 0.007% weight by volume (w/v) EVT. In embodiments, the biological sample includes less than 0.006% weight by volume (w/v) EVT. In embodiments, the biological sample includes less than 0.005% weight by volume (w/v) EVT. In embodiments, the biological sample includes less than 0.004% weight by volume (w/v) EVT. In embodiments, the biological sample includes less than 0.003% weight by volume (w/v) EVT. In embodiments, the biological sample includes less than 0.002% weight by volume (w/v) EVT. In embodiments, the biological sample includes less than 0.001% weight by volume (w/v) EVT.

In embodiments, the biological sample includes at least about 0.001% weight by volume (w/v) EVT. In embodiments, the biological sample includes at least about 0.002% weight by volume (w/v) EVT. In embodiments, the biological sample includes at least about 0.003% weight by volume (w/v) EVT. In embodiments, the biological sample includes at least about 0.004% weight by volume (w/v) EVT. In embodiments, the biological sample includes at least about 0.005% weight by volume (w/v) EVT. In embodiments, the biological sample includes at least about 0.006% weight by volume (w/v) EVT. In embodiments, the biological sample includes at least about 0.007% weight by volume (w/v) EVT. In embodiments, the biological sample includes at least about 0.008% weight by volume (w/v) EVT. In embodiments, the biological sample includes at least about 0.009% weight by volume (w/v) EVT. In embodiments, the biological sample includes at least about 0.01% weight by volume (w/v) EVT. In embodiments, the biological sample includes at least about 0.02% weight by volume (w/v) EVT. In embodiments, the biological sample includes at least about 0.03% weight by volume (w/v) EVT. In embodiments, the biological sample includes at least about 0.04% weight by volume (w/v) EVT. In embodiments, the biological sample includes at least about 0.05% weight by volume (w/v) EVT. In embodiments, the biological sample includes at least about 0.06% weight by volume (w/v) EVT. In embodiments, the biological sample includes at least about 0.07% weight by volume (w/v) EVT. In embodiments, the biological sample includes at least about 0.08% weight by volume (w/v) EVT. In embodiments, the biological sample includes at least about 0.09% weight by volume (w/v) EVT. In embodiments, the biological sample includes at least about 0.1% weight by volume (w/v) EVT. In embodiments, the biological sample includes at least about 0.2% weight by volume (w/v) EVT. In embodiments, the biological sample includes at least about 0.3% weight by volume (w/v) EVT. In embodiments, the biological sample includes at least about 0.4% weight by volume (w/v) EVT. In embodiments, the biological sample includes at least about 0.5% weight by volume (w/v) EVT. In embodiments, the biological sample includes at least about 0.6% weight by volume (w/v) EVT. In embodiments, the biological sample includes at least about 0.7% weight by volume (w/v) EVT. In embodiments, the biological sample includes at least about 0.8% weight by volume (w/v) EVT. In embodiments, the biological sample includes at least about 0.9% weight by volume (w/v) EVT. In embodiments, the biological sample includes at least about 1% weight by volume (w/v) EVT.

In embodiments, the biological sample includes at least about 1% EVT cells, as measured by mass percentage of solute in solution (w/w). In embodiments, the biological sample includes at least about 1%, at least about 2%, at least about 3%, at least about 4%, at least about 5%, at least about 6%, at least about 7%, at least about 8%, at least about 9%, at least about 10%, at least about 12%, at least about 14%, at least about 16%, at least about 18%, at least about 20%, at least about 30%, at least about 40%, at least about 50%, at least about 60%, at least about 70%, at least about 80%, at least about 80%, at least about 90% or at least about 100% EVT cells, as measured by mass percentage of solute in solution (w/w).

In embodiments, the biological sample includes at most about 100%, at most about 90%, at most about 80%, at most about 70%, at most about 60%, at most about 50%, at most about 40%, at most about 30%, at most about 20%, at most about 18%, at most about 16%, at most about 14%, at most about 12%, at most about 10%, at most about 9%, at most about 8%, at most about 7%, at most about 6%, at most about 5%, at most about 4%, at most about 3%, at most about 2%, or at most about 1% EVT cells, as measured by mass percentage of solute in solution (w/w).

In embodiments, the biological sample includes at least about 1% EVT, as measured by, as measured by volume by volume percentage (v/v). In embodiments, the biological sample includes at least about 1%, at least about 2%, at least about 3%, at least about 4%, at least about 5%, at least about 6%, at least about 7%, at least about 8%, at least about 9%, at least about 10%, at least about 12%, at least about 14%, at least about 16%, at least about 18%, at least about 20%, at least about 30%, at least about 40%, at least about 50%, at least about 60%, at least about 70%, at least about 80%, at least about 80%, at least about 90% or at least about 100% EVT cells, as measured by volume by volume percentage (v/v).

In embodiments, the biological sample includes at most about 100%, at most about 90%, at most about 80%, at most about 70%, at most about 60%, at most about 50%, at most about 40%, at most about 30%, at most about 20%, at most about 18%, at most about 16%, at most about 14%, at most about 12%, at most about 10%, at most about 9%, at most about 8%, at most about 7%, at most about 6%, at most about 5%, at most about 4%, at most about 3%, at most about 2%, or at most about 1% EVT cells, as measured by volume by volume percentage (v/v).

In embodiments, the biological sample includes at least about 0.00005%, at least about 0.0001%, at least about 0.0005%, at least about 0.001%, at least about 0.005%, at least about 0.01%, at least about 0.05%, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 12%, at least about 14%, at least about 16%, at least about 18%, or at least about 20% EVT cells, as measured by weight by volume percentage (w/v).

In embodiments, the biological sample includes at most about 20%, at most about 18%, at most about 16%, at most about 14%, at most about 12%, at most about 10%, at most about 9%, at most about 8%, at most about 7%, at most about 6%, at most about 5%, at most about 4%, at most about 3%, at most about 2%, at most about 1%, at most about 0.5%, at most about 0.1%, at most about 0.05%, at most about 0.01%, at most about 0.005%, at most about 0.001% at most about 0.0005%, at most about 0.0001%, or at most about 0.00005% EVT cells, as measured by weight by volume percentage (w/v).

In embodiments, the biological sample includes at least about 1×10⁻¹⁵ grams, at least about 9.5×10⁻¹⁴ grams, at least about 9×10⁻¹⁴ grams, at least about 8.5×10⁻¹⁴ grams, at least about 8×10⁻¹⁴ grams, at least about 7.5×10⁻¹⁴ grams, at least about 7×10⁻¹⁴ grams, at least about 6.5×10⁻¹⁴ grams, at least about 6×10⁻¹⁴ grams, at least about 5.5×10⁻¹⁴ grams, at least about 5×10⁻¹⁴ grams, at least about 4.5×10⁻¹⁴ grams, at least about 4×10⁻¹⁴ grams, at least about 3.5×10⁻¹⁴ grams, at least about 3×10⁻¹⁴ grams, at least about 2.8×10⁻¹⁴ grams, at least about 2.6×10⁻¹⁴ grams, at least about 2.4×10⁻¹⁴ grams, at least about 2.2× 10-14 grams, at least about 2×10⁻¹⁴ grams, at least about 1.8×10⁻¹⁴ grams, at least about 1.6×10⁻¹⁴ grams, at least about 1.4×10⁻¹⁴ grams, at least about 1.2×10⁻¹⁴ grams, at least about 1×10⁻¹⁴ grams, or at least about 1×10⁻¹³ grams, of cEVT cells, when measured in wet weight per volume.

In embodiments, the method further includes washing the biological sample to achieve a single-cell solution. In embodiments, the method further includes filtering the biological sample to achieve a single-cell solution. For example, in embodiments, the method further includes a step of isolating a single EVT cell.

In embodiments, the at least one biomarker is derived from a placenta-specific cell, a maternal cell, or a fetal cell. In embodiments, the at least one biomarker is derived from a placenta-specific cell. In embodiments, the at least one biomarker is derived from a maternal cell. In embodiments, the at least one biomarker is derived from a fetal cell. In embodiments, the placenta-specific cell is an extravillous trophoblast (EVT), villous trophoblast or syncytiotrophoblast cell. In embodiments, the placenta-specific cell is an extravillous trophoblast (EVT). In embodiments, the placenta-specific cell is a villous trophoblast. In embodiments, the placenta-specific cell is a syncytiotrophoblast cell.

In embodiments, the at least one biomarker includes a placental growth factor (PGF), pregnancy-associated plasma protein-A (PAPP-A), galectin 13 (LGALS13), galectin 14 (LGALS14), alpha fetoprotein (AFP), endoglin (ENG), fms-like tyrosine kinase 1 (FLT), CGB, ADAM12, ADAM17, BDNF, CCL5, CRP, CXCL8, EGF, sEGFR, EPO, HBEGF, IFNG, IGF, IGFBP1, ILIB, IL6, INHA, MMP2, MMP7, MMP9, MMP12, NGF, TGA1, TGFA, TGFB2, TIMP3, TNFA, TSH, VEGF, or a combination thereof.

As described throughout the specification, the methods provided herein allow for detection of biomarkers expressed by EVT cells. For example, the CyTOF-MS methods provided herein allow detection of EVT biomarkers in a biological sample including EVT cells by contacting the biological sample with one or more antibodies specific for a an EVT biomarker. For example, the biomarker may be a protein expressed on the surface of an EVT biomarker. In another example, the biomarker may be an intracellular protein expressed by an EVT cell. Thus, in embodiments, the at least one biomarker includes an EVT biomarker. EVT cell biomarkers are biomarkers expressed by EVT cells. The detection of an increased or decreased level of an EVT biomarker may be associated with a particular physical condition or state. For example, an increased or decreased level of an EVT biomarker may be associated a pregnancy-associated risk or condition. In embodiments, the EVT cell biomarker includes integrin subunit alpha 1 (ITGA1), cadherin 5 (CDH5), platelet and endothelial cell adhesion (PECAM1), matrix metallopeptidase 9 (MMP9), HLA-G, integrin alpha 6 (ITGA6), chorionic gonadotropin (hCG), and pregnancy-specific beta-1-glycoprotein 1 (PSG1), or a combination thereof.

In embodiments, the at least one biomarker includes a placental protein biomarker. In embodiments, the placental protein biomarker includes hCG, progesterone, human placental lactogen, vascular endothelial growth factor (VEGF), VEGF-receptor (FLT1), PGF, insulin-like growth factor binding protein 1 (IGFBP1 or PP12), galectin 13 (LGALS13), galectin 14 (LGALS14), vasculotropin, PAPPA, endoglin (ENG), vascular endothelial cell proliferation factor, or a combination thereof.

In embodiments, the at least one biomarker includes a biomarker which identifies a marker of cervical health. Cervical health-related biomarkers are biomarkers which are associated with various cervix-related conditions such as cervical infection, bleeding, inflammation, and cervical cancer. Thus, in embodiments, the method further includes identifying a cervical condition. In embodiments, the cervical condition is cervical infection; bleeding; inflammation, or cervical cancer. In embodiments, the cervical health related biomarker includes carcinoembryonic antigen (CEA), squamous cell carcinoma antigen (SCC Ag) and carbohydrate antigen 19-9 (CA19-9) or a combination thereof. In embodiments, the cervical health related biomarker includes HCGB, KRT7, hPL, CDH5, PECAM1, ITGA1, MMP9, TGFB2, HLAG, PSG1, ITGA6, CDH1, LGALS13, LGALS14, PAPPA, PGF, AFP, FLT, ENG, Spint1, ADAM12, MPO, CD68, BMK13, CD45, or a combination thereof.

In embodiments, the one or more pregnancy-associated risks or conditions include placental dysfunction or insufficiency, pregnancy-induced hypertension, placental abruption, pregnancy loss, miscarriage, preeclampsia, eclampsia, Hemolysis Elevated Liver enzymes and Low Platelet (HELLP) syndrome, fetal growth restriction, intrauterine growth restriction, preterm birth, low birthweight, placenta percreta, placenta increta, placenta previa, gestational hypertension, gestational thrombosis, stillbirth, placental infarction, or a combination thereof. In embodiments, the one or more pregnancy-associated risks or conditions includes an early gestational complication. In embodiments, the early gestational complication is placental dysfunction or insufficiency, risk of early pregnancy loss, risk of preeclampsia, or risk of preterm birth.

As described herein, the methods provided herein allow for early detection of pregnancy-associated risks, conditions, or states. Thus, for the methods provided herein, in embodiments, the subject is a pregnant subject. In embodiments, the subject is at least 4 weeks pregnant (e.g. as measured by gestational age). In embodiments, the subject is at least 5 weeks pregnant. In embodiments, the subject is at least 6 weeks pregnant. In embodiments, the subject is at least 7 weeks pregnant. In embodiments, the subject is at least 8 weeks pregnant. In embodiments, the subject is at least 9 weeks pregnant. In embodiments, the subject is at least 10 weeks pregnant.

As described throughout the specification, methods provided herein allow for accurate and sensitive detection of altered (e.g. elevated or decreased) levels of at least one biomarker obtained from a biological sample obtained from the cervix of a subject. For example, the methods allow for detection of biomarkers expressed or produced by EVT cells in biological samples including less than 10% (w/v) EVT. Thus, in an aspect is provided a method of identifying one or more pregnancy-associated risks or conditions in a subject, the method including: a) obtaining a biological sample from the cervix of the subject, wherein the biological sample includes extravillous trophoblast (EVT) cells and biological material derived from the cervix of the subject, wherein the biological material includes at least 90% weight by volume (w/v) of the biological sample; b) performing single-cell time-of-flight mass spectrometry (CyTOF-MS) on the biological sample to generate an output; and c) determining an elevated level or a decreased level of at least one biomarker in the biological sample relative to a standard control based on the output, thereby identifying the one or more pregnancy-associated risks or conditions.

As described above, Applicant has discovered a method for sensitive and accurate detection of an elevated level or decreased level of a biomarker in a biological sample including EVT cells. The biomarker may include a lipid, carbohydrate, DNA or protein expressed by or produced by an EVT cell. In embodiments, the biomarker is attached to the surface of the EVT cell. In embodiments, the method does not include isolation, purification, or enrichment of EVT cells from the biological sample. For example, an elevated level or decreased level of a biomarker may be detected in the biological sample using CyTOF-MS without isolating the EVT cells. Thus, in embodiments, the method does not include isolating the EVT. In embodiments, the method provided herein allows for sensitive and accurate detection of an elevated level or decreased level of a biomarker using CyTOF-MS in a biological sample without enrichment of EVT.

As described throughout the specification, for the method provided herein, in embodiments the elevated level or decreased level of at least one biomarker is measured by mass spectrometry. For the methods provided herein, in embodiments, mass spectrometry may be used in combination with one or more additional methods (e.g. flow cytometry) for identifying the at least one biomarker provided herein. In embodiments, the method further includes performing flow cytometry on the biological sample.

For the method provided herein, in embodiments, the method is completed within 24 hours. In embodiments, CyTOF-MS and/or flow cytometry is performed on the biological sample within 24 hours of obtaining the biological sample from the subject. In embodiments, CyTOF-MS and/or flow cytometry is performed within 2 hours, within 3 hours, within 4 hours, within 5 hours, within 6 hours, within 7 hours, within 8 hours, within 9 hours, within 10 hours, within 11 hours, within 12 hours, within 13 hours, within 14 hours, within 15 hours, within 16 hours, within 17 hours, within 18 hours, within 19 hours, within 20 hours, within 21 hours, within 22 hours, within 23 hours, or within 24 hours of obtaining the biological sample from the object.

For the method provided herein, in embodiments, the biological sample includes at least about 25 cells per 2 milliliters volume. In embodiments, the biological sample includes less than 0.1% weight by volume (w/v) EVT.

For the methods provided herein, in embodiments, the biological sample is collected through a pap smear or other procedure that allows for the collection of biological sample from the cervix (e.g., a diva cup). In embodiments, the biological sample includes biological material derived from the subject (e.g., maternal cells). In embodiments, the biological sample includes biological material derived from the fetus/placenta (e.g., fetal cells or placental cells).

For the methods provided herein, in embodiments, the biological sample includes components (e.g. biological material (e.g. mucous, cells, blood, DNA, protein, etc.)). that have transferred to the cervix from elsewhere in the subject's body. For example, the biological sample may include components (e.g. biological material) derived from the uterus and/or placenta of the subject which have accumulated in the cervix. In embodiments, the biological sample includes cells that have accumulated in the cervix (e.g., EVT cells or other placental cells). Thus, in embodiments, the EVT include EVT residing in or passing through the cervix. In embodiments, a cervical sample includes other body fluids or biological materials such as mucous, blood, or fetal DNA. In embodiments, the biological sample includes placental cells.

In embodiments, the method further includes washing the biological sample to achieve a single-cell solution. In embodiments, the method further includes filtering the biological samples to achieve a single-cell solution.

In embodiments, the at least one biomarker is derived from a placenta-specific cell, a maternal cell, or a fetal cell. In embodiments, the at least one biomarker is derived from a placenta-specific cell. In embodiments, the at least one biomarker is derived from a maternal cell. In embodiments, the at least one biomarker is derived from a fetal cell. In embodiments, the placenta-specific cell is an extravillous trophoblast (EVT), villous trophoblast or syncytiotrophoblast cell. In embodiments, the placenta-specific cell is an extravillous trophoblast (EVT). In embodiments, the placenta-specific cell is a villous trophoblast. In embodiments, the placenta-specific cell is a syncytiotrophoblast cell.

In embodiments, the at least one biomarker includes a placental growth factor (PGF), pregnancy-associated plasma protein-A (PAPP-A), galectin 13 (LGALS13), galectin 14 (LGALS14), alpha fetoprotein (AFP), endoglin (ENG), fms-like tyrosine kinase 1 (FLT), CGB, ADAM12, ADAM17, BDNF, CCL5, CRP, CXCL8, EGF, SEGFR, EPO, HBEGF, IFNG, IGF, IGFBP1, ILIB, IL6, INHA, MMP2, MMP7, MMP9, MMP12, NGF, TGA1, TGFA, TGFB2, TIMP3, TNFA, TSH, VEGF, or a combination thereof.

In embodiments, the at least one biomarker includes an EVT biomarker. In embodiments, the EVT cell biomarker includes integrin subunit alpha 1 (ITGA1), cadherin 5 (CDH5), platelet and endothelial cell adhesion (PECAM1), matrix metallopeptidase 9 (MMP9), HLA-G, integrin alpha 6 (ITGA6), chorionic gonadotropin (hCG), and pregnancy-specific beta-1-glycoprotein 1 (PSG1), or a combination thereof.

In embodiments, the at least one biomarker includes a placental protein. In embodiments, the placental protein includes hCG, progesterone, human placental lactogen, vascular endothelial growth factor (VEGF), VEGF-receptor (FLT1), PGF, insulin-like growth factor binding protein 1 (IGFBP1 or PP12), galectin 13 (LGALS13), galectin 14 (LGALS14), vasculotropin, PAPPA, endoglin (ENG), vascular endothelial cell proliferation factor, or a combination thereof.

In embodiments, the at least one biomarker comprises a biomarker which identifies a marker of cervical health. Cervical health-related biomarkers are biomarkers which are associated with various cervix-related conditions such as cervical infection, bleeding, inflammation, and cervical cancer. In embodiments, the cervical health related biomarker includes carcinoembryonic antigen (CEA), squamous cell carcinoma antigen (SCC Ag) and carbohydrate antigen 19-9 (CA19-9) or a combination thereof. In embodiments, the cervical health related biomarker includes HCGB, KRT7, hPL, CDH5, PECAM1, ITGA1, MMP9, TGFB2, HLAG, PSG1, ITGA6, CDH1, LGALS13, LGALS14, PAPPA, PGF, AFP, FLT, ENG, Spint1, ADAM12, MPO, CD68, BMK13, CD45, or a combination thereof. Thus, in embodiments, the method further includes identifying a cervical condition. In embodiments, the cervical condition is cervical infection; bleeding; inflammation, or cervical cancer. In embodiments, the cervical infection is human papilloma virus infection.

In embodiments, the one or more pregnancy-associated risks or conditions includes placental dysfunction or insufficiency, pregnancy-induced hypertension, placental abruption, pregnancy loss, miscarriage, preeclampsia, eclampsia, Hemolysis Elevated Liver enzymes and Low Platelet (HELLP) syndrome, fetal growth restriction, intrauterine growth restriction, preterm birth, low birthweight, placenta percreta, placenta increta, placenta previa, gestational hypertension, gestational thrombosis, stillbirth, placental infarction, or a combination thereof.

In embodiments, the presence or absence of the at least one biomarker is indicative of an early gestational complication. Thus, in embodiments, the one or more pregnancy-associated risks or conditions comprises an early gestational complication. Early gestational complications can include, but are not limited to, malplacentation, placental dysfunction, placental insufficiency, pregnancy loss, preeclampsia, and fetal growth restriction. In embodiments, the early gestational complication is placental dysfunction or insufficiency, risk of early pregnancy loss, risk of preeclampsia, or risk of preterm birth.

### Kits

Provided herein, inter alia, is a kit for detecting increased or decreased levels of biomarkers indicative of a pregnancy-associated risk or condition in a cervical fluid sample obtained from a subject, wherein the cervical fluid sample includes 0 to 2 cells per 2 mL volume. Detection of biomarkers in a substantially cell free (e.g. less than 3 cells, less than 2 cells, or less than 1 cell per 2 mL volume) cervical fluid samples allows for minimally invasive screening of pregnant subjects. Further, detection of biomarkers in a substantially cell free sample is contemplated to have higher accuracy compared to detection methods in cell-containing biological samples (e.g. blood samples). Thus, in an aspect is provided a kit for obtaining a cervical fluid sample from a subject, including: a) a collection device; b) a collection container including a stabilizing solution; and c) a cell lysis solution and/or a cell removal device.

In embodiments, the kit includes a device for collecting the cervical fluid sample. In embodiments, the device for collecting the cervical fluid sample is a cytobrush or cytological brush. In embodiments, the device for collecting the cervical fluid includes a handle and a molded silicon tip for obtaining a cervical fluid sample. In embodiments, the device for collecting the cervical fluid sample includes a catheter. For example, the catheter may be used to suction and collect the cervical fluid sample from the cervix. In embodiments, the device for collecting the cervical fluid sample includes a lavage device that deposits saline into the cervical canal and subsequently obtains the saline and the cervical fluid sample. In embodiments, the device for collecting the cervical fluid sample includes a menstrual cup or menstrual disc. For example, the menstrual cup or disc can be inserted below the cervix for the cervical fluid sample to accumulate within the cup or disc.

In embodiments, the kit includes a collection vial for the cervical fluid sample. In embodiments, the collection vial includes a cell fixative. The cell fixative may prevent degradation or lysis of cells in the cervical fluid sample. The cell fixative may include methanol, ethanol, formaldehyde, glutaraldehyde or any fixative known to one skilled in the biological arts. In embodiments, the collection vial includes a stabilization solution. In embodiments, the stabilization solution is a protein stabilizing solution or a nucleic acid stabilizing solution. In another example, the stabilizing solution inhibits degradation of proteins and/or nucleic acids. In another embodiment, the kit includes a cell lysis agent and a protein stabilizing solution or a nucleic acid stabilizing solution.

In embodiments, the cell lysis agent is a nonionic detergent. In another embodiment, the cells in the cervical fluid sample are not contacted with a cell lysis agent and are removed from the cervical fluid sample. In embodiments, cells are removed from the cervical fluid sample by centrifugation or filtration. For example, the cell may be removed from the cervical fluid sample by centrifugation at 200×g to 800×g. For example, cell may be filtered through a device with pores sizes between 0.1 and 10 microns.

In embodiments, the kit includes an agent for removing mucus from the cervical fluid sample. In embodiments, the kit includes a mucus solubilizing agent. For example, the mucus solubilizing agent may be a reducing agent or a glycosidase.

In embodiments, the kit includes a device for filtration of the cervical fluid sample. In embodiments, the kit includes a device for centrifuging the cervical fluid sample. In embodiments, the kit includes a device using microfluidic technology for removing cells or mucous from the cervical fluid sample. In embodiments, the kit includes a cell removal device. In embodiments, the kit includes a cell lysis solution. In embodiments, the kit includes instructions for obtaining the cervical fluid sample.

In embodiments, the kit further includes a detection assay for detecting an elevated level or a decreased level of at least one biomarker in the cervical fluid sample. In embodiments, the detection assay is an enzyme-linked immunosorbent assay (ELISA), gel electrophoresis, western blotting, mass spectrometry, capillary electrophoresis, protein sequencing, polymerase chain reaction (PCR), digital PCR, DNA sequencing using capillary electrophoresis or next-generation sequencing (NGS), reverse transcription of RNA followed by PCR, digital PCR or NGS, liquid chromatography, thin layer chromatography, or a combination thereof. In embodiments, the detection assay includes a lateral flow device. In embodiments, the kit further includes regents (e.g. detectable agents, antibodies, etc.) for the detection assay. In embodiments, the kit includes instructions for the detection assay.

In embodiments, the at least one biomarker is a protein, nucleic acid, cell fragment, microvesicle, ectosome, microparticle, extracellular vesicle, micelle, or combination thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Embodiments

P1 Embodiment 1. A method of identifying one or more pregnancy-associated risks or conditions of a subject, the method comprising: a) obtaining a biological sample from a cervix of the subject, the biological sample comprising extravillous trophoblast (EVT) cells and biological materials derived from the cervix of the subject, wherein the biological materials derived from the cervix of the subject comprise at least 90% of the biological sample; b) performing single-cell time-of-flight mass spectrometry (CyTOF-MS) on the biological sample to generate an output; and c) determining the presence or absence of at least one biomarker in the biological sample based on the output, wherein the presence or absence of the at least one biomarker is indicative of an early gestational complication.

P1 Embodiment 2. A method of identifying one or more pregnancy-associated risks or conditions of a subject, the method comprising: a) obtaining a biological sample from a cervix of the subject, the biological sample comprising extravillous trophoblast (EVT) cells; b) performing single-cell time-of-flight mass spectrometry (CyTOF-MS) on the biological sample to generate an output; and c) determining the presence or absence of at least one biomarker in the biological sample based on the output.

P1 Embodiment 3. The method of P1 embodiment 2, wherein the biological sample further comprises biological materials derived from the cervix of the subject.

P1 Embodiment 4. The method of P1 embodiment 3, wherein the biological materials derived from the cervix are naturally present in the cervix during pregnancy.

P1 Embodiment 5. The method of any one of P1 embodiments 2-4, wherein the biological materials derived from the cervix comprise mucous, maternal cells, a biological fluid, or any combination thereof.

P1 Embodiment 6. The method of any one of P1 embodiments 2-5, wherein the biological materials derived from the cervix comprise at least 90% of the biological sample.

P1 Embodiment 7. The method of any one of P1 embodiments 2-6, further comprising washing the biological sample to achieve a single-cell solution.

P1 Embodiment 8. The method of any one of P1 embodiments 2-7, further comprising filtering the biological samples to achieve a single-cell solution.

P1 Embodiment 9. The method of any one of P1 embodiments 2-8, wherein the at least one biomarker comprises a biomarker which identifies EVT cells in the biological sample.

P1 Embodiment 10. The method of any one of P1 embodiments 2-9, wherein the at least one biomarker comprises a biomarker which identifies placental-related proteins.

P1 Embodiment 11. The method of any one of P1 embodiments 2-8, wherein the at least one biomarker comprises a biomarker which identifies a marker of cervical health.

P1 Embodiment 12. The method of P1 Embodiment 11, wherein the marker of cervical health can be selected from the group consisting of presence of cervical infection; bleeding; inflammation, and cervical cancer.

P1 Embodiment 13. The method of any one of P1 embodiments 2-12, wherein the presence or absence of the at least one biomarker is indicative of an early gestational complication.

P1 Embodiment 14. The method of P1 Embodiment 13, wherein the early gestational complication is placental dysfunction or insufficiency.

P1 Embodiment 15. The method of P1 Embodiment 13, wherein the early gestational complication is a risk of early pregnancy loss.

P1 Embodiment 16. The method of P1 Embodiment 13, wherein the early gestational complication is a risk of preeclampsia.

P1 Embodiment 17. The method of P1 Embodiment 13, wherein the early gestational complication is a risk of preterm birth.

P1 Embodiment 18. The method of any one of P1 embodiments 2-16, wherein the EVT purity of the sample is less than 0.1%.

P1 Embodiment 19. The method of any one of P1 embodiments 2-18, wherein the subject is a pregnant subject.

P1 Embodiment 20. The method of any one of P1 embodiments 2-19, wherein the sample is taken from a pregnant subject that is at least five weeks pregnant.

P1 Embodiment 21. The method of any one of P1 embodiments 2-20, wherein the sample consists of at least about 25 cells.

P1 Embodiment 22. The method of any one of P1 embodiments 2-21, wherein the method is completed in less than 24 hours.

P1 Embodiment 23. The method of any one of P1 embodiments 2-22, wherein the EVT cells comprise extravillous cells residing in or passing through the cervix.

P1 Embodiment 24. The method of any one of P1 embodiments 2-23, further comprising performing flow cytometry analysis on the biological sample.

P1 Embodiment 25. A kit used to obtain the biological sample of any one of P1 embodiments 2-24, wherein the kit comprises: a) a biohazard spill-proof bag, b) a scraper, c) a cyto-brush, and d) a container comprising a stabilizing solution.

P2 Embodiment 1. A method of identifying a pregnancy-associated risk or condition in a subject, the method comprising: a) obtaining a cervical fluid sample from a subject; and b) detecting an elevated level or a decreased level of at least one biomarker in the cervical fluid sample relative to a standard control, thereby identifying the pregnancy associated risk or condition, wherein the cervical fluid sample comprises 0 to 2 cells per 2 ml volume.

P2 Embodiment 2. The method of P2 embodiment 1, wherein the cervical fluid sample is derived from a cervical secretion, a cervical mucous, a cervical emission, cervical excretion, or cervical discharge.

P2 Embodiment 3. The method of P2 embodiment 1 or 2, wherein the cervical fluid sample is cell free or substantially cell free.

P2 Embodiment 4. The method of any one of P2 embodiments 1 to 3, wherein the at least one biomarker is derived from a placenta-specific cell, a maternal cell, or a fetal cell.

P2 Embodiment 5. The method of P2 embodiment 4, wherein the placenta-specific cell is an extravillous trophoblast (EVT), villous trophoblast or syncytiotrophoblast cell.

P2 Embodiment 6. The method of any one of P2 embodiments 1 to 5, wherein the at least one biomarker is a protein, nucleic acid, cell fragment, microvesicle, ectosome, microparticle, extracellular vesicle, micelle, or combination thereof.

P2 Embodiment 7. The method of P2 embodiment 6, wherein the protein is placental growth factor (PGF), pregnancy-associated plasma protein-A (PAPP-A), galectin 13 (LGALS13), galectin 14 (LGALS14), alpha fetoprotein (AFP), endoglin (ENG), fms-like tyrosine kinase 1 (FLT), CGB, ADAM12, ADAM17, BDNF, CCL5, CRP, CXCL8, EGF, SEGFR, EPO, HBEGF, IFNG, IGF, IGFBP1, IL1B, IL6, INHA, MMP2, MMP7, MMP9, MMP12, NGF, TGA1, TGFA, TGFB2, TIMP3, TNFA, TSH, VEGF, or a fragment thereof.

P2 Embodiment 8. The method of P2 embodiment 7, wherein the protein is PGF, PAPP-A, LGALS13, LGALS14, AFP, ENG, FLT, or a fragment thereof.

P2 Embodiment 9. The method of any one of P2 embodiments 1 to 8, wherein the detecting comprises performing an immunoassay, mass spectrometry, a polymerase chain reaction (PCR), a sequencing method, or a combination thereof.

P2 Embodiment 10. The method of any one of P2 embodiments 1 to 9, wherein the pregnancy-associated risk or condition is placental insufficiency, pregnancy-induced hypertension, placental abruption, pregnancy loss, miscarriage, preeclampsia, eclampsia, Hemolysis Elevated Liver enzymes and Low Platelet (HELLP) syndrome, fetal growth restriction, intrauterine growth restriction, preterm birth, low birthweight, or a combination thereof.

P2 Embodiment 11. A kit for obtaining a cervical fluid sample from a subject, comprising: a) a collection device; b) a collection container comprising a stabilizing solution; and c) a cell lysis solution and/or a cell removal device.

P2 Embodiment 12. The kit of P2 embodiment 11, further comprising: a protein stabilizing solution, or a nucleic acid stabilizing solution.

P2 Embodiment 13. The kit of P2 embodiment 11 or 12, further comprising a mucous solubilizing agent, or a mucous removal device.

P2 Embodiment 14. The kit of any one of P2 embodiments 11 to 13, further comprising a detection assay for detecting an elevated level or a decreased level of at least one biomarker in the cervical fluid sample.

P2 Embodiment 15. The kit of P2 embodiment 14, wherein the detection assay comprises a lateral flow device.

P2 Embodiment 16. The kit of P2 embodiment 14 or 15, wherein the at least one biomarker is a protein, nucleic acid, cell fragment, microvesicle, ectosome, microparticle, extracellular vesicle, micelle, or combination thereof.

Embodiment 1. A method of identifying a pregnancy-associated risk or condition in a subject, the method comprising: a) obtaining a cervical fluid sample from a subject; and b) detecting an elevated level or a decreased level of at least one biomarker in the cervical fluid sample relative to a standard control, thereby identifying the pregnancy associated risk or condition, wherein the cervical fluid sample comprises no greater than 1 cell per 1 milliliter (mL) volume.

Embodiment 2. The method of embodiment 1, wherein the cervical fluid sample is derived from a cervical secretion, a cervical mucous, a cervical emission, cervical excretion, or cervical discharge.

Embodiment 3. The method of embodiment 1 or 2, wherein the cervical fluid sample is cell free or substantially cell free.

Embodiment 4. The method of any one of embodiments 1 to 3, wherein the at least one biomarker is derived from a placenta-specific cell, a maternal cell, or a fetal cell.

Embodiment 5. The method of embodiment 4, wherein the placenta-specific cell is an extravillous trophoblast (EVT), villous trophoblast or syncytiotrophoblast cell.

Embodiment 6. The method of any one of embodiments 1 to 5, wherein the at least one biomarker is a protein, nucleic acid, cell fragment, microvesicle, ectosome, microparticle, extracellular vesicle, micelle, or combination thereof.

Embodiment 7. The method of embodiment 6, wherein the protein is placental growth factor (PGF), pregnancy-associated plasma protein-A (PAPP-A), galectin 13 (LGALS13), galectin 14 (LGALS14), alpha fetoprotein (AFP), endoglin (ENG), fms-like tyrosine kinase 1 (FLT), CGB, ADAM12, ADAM17, BDNF, CCL5, CRP, CXCL8, EGF, SEGFR, EPO, HBEGF, IFNG, IGF, IGFBP1, IL1B, IL6, INHA, MMP2, MMP7, MMP9, MMP12, NGF, TGA1, TGFA, TGFB2, TIMP3, TNFA, TSH, VEGF, or a combination thereof.

Embodiment 8. The method of embodiment 7, wherein the protein is PGF, PAPP-A, LGALS13, LGALS14, AFP, ENG, FLT, or a combination thereof.

Embodiment 9. The method of any one of embodiments 1 to 8, wherein the at least one biomarker comprises an EVT biomarker.

Embodiment 10. The method of embodiment 9, wherein the EVT biomarker comprises integrin subunit alpha 1 (ITGA1), cadherin 5 (CDH5), cadherin 1 (CDH1), platelet and endothelial cell adhesion (PECAM1), matrix metallopeptidase 9 (MMP9), HLA-G, integrin alpha 6 (ITGA6), chorionic gonadotropin (hCG), pregnancy-specific beta-1-glycoprotein 1 (PSG1), or a combination thereof.

Embodiment 11. The method of any one of embodiments 1 to 10, wherein the at least one biomarker comprises a placental protein.

Embodiment 12. The method of embodiment 11, wherein the placental protein comprises hCG, progesterone, human placental lactogen, vascular endothelial growth factor (VEGF), VEGF-receptor (FLT1), PGF, insulin-like growth factor binding protein 1 (IGFBP1 or PP12), galectin 13 (LGALS13), galectin 14 (LGALS14), vasculotropin, PAPPA, endoglin (ENG), vascular endothelial cell proliferation factor, or a combination thereof.

Embodiment 13. The method of any one of embodiments 1 to 12, wherein the detecting comprises performing an immunoassay, mass spectrometry, a polymerase chain reaction (PCR), a sequencing method, or a combination thereof.

Embodiment 14. The method of any one of embodiments 1 to 13, further comprising identifying a cervical condition.

Embodiment 15. The method of embodiment 14, wherein identifying a cervical condition comprises determining an elevated level or a decreased level of at least one cervical health biomarker relative to a standard control.

Embodiment 16. The method of embodiment 15, wherein the at least one cervical health biomarker comprises carcinoembryonic antigen (CEA), squamous cell carcinoma antigen (SCC Ag) carbohydrate antigen 19-9 (CA19-9), KRT7, hPL, CDH5, PECAM1, ITGA1, MMP9, TGFB2, HLAG, PSG1, ITGA6, CDH1, LGALS13, LGALS14, PAPPA, PGF, AFP, FLT, ENG, Spint1, ADAM12, MPO, CD68, BMK13, CD45, or a combination thereof.

Embodiment 17. The method of any one of embodiments 14 to 16, wherein the cervical condition is cervical infection, bleeding, inflammation, or cervical cancer.

Embodiment 18. The method of embodiment 17, wherein the cervical infection is human papilloma virus infection.

Embodiment 19. The method of any one of embodiments 1 to 18, wherein the pregnancy-associated risk or condition comprises placental insufficiency, pregnancy-induced hypertension, placental abruption, pregnancy loss, miscarriage, preeclampsia, eclampsia, Hemolysis Elevated Liver enzymes and Low Platelet (HELLP) syndrome, fetal growth restriction, intrauterine growth restriction, preterm birth, low birthweight, placenta percreta, placenta increta, placenta previa, gestational hypertension, gestational thrombosis, stillbirth, placental infarction, or a combination thereof.

Embodiment 20. The method of any one of embodiments 1 to 19, wherein the one or more pregnancy-associated risks or conditions comprises an early gestational complication.

Embodiment 21. The method of embodiment 20, wherein the early gestational complication is placental dysfunction or insufficiency, risk of early pregnancy loss, risk of preeclampsia, risk of preterm birth, or risk of gestational diabetes.

Embodiment 22. A method of identifying one or more pregnancy-associated risks or conditions in a subject, the method comprising: a) obtaining a biological sample from the cervix of the subject, wherein the biological sample comprises extravillous trophoblast (EVT) cells; b) performing single-cell time-of-flight mass spectrometry (CyTOF-MS) on the biological sample to generate an output; and c) determining an elevated level or a decreased level of at least one biomarker in the biological sample relative to a standard control based on the output.

Embodiment 23. The method of embodiment 22, wherein the method does not comprise isolating the EVT cells.

Embodiment 24. The method of embodiment 22 or 23, further comprising performing flow cytometry on the biological sample.

Embodiment 25. The method of any one of embodiments 22 to 24, wherein the method is completed in less than 24 hours.

Embodiment 26. The method of any one of embodiments 22 to 25, wherein the biological sample further comprises biological material derived from the cervix of the subject.

Embodiment 27. The method of embodiment 26, wherein the biological material comprises mucous, a maternal cell, biological fluid, or a combination thereof.

Embodiment 28. The method of embodiment 26 or 27, wherein the biological material comprises at least 90% weight by volume (w/v) of the biological sample.

Embodiment 29. The method of any one of embodiments 22 to 28, wherein the biological sample comprises at least about 25 cells per 2 mL volume.

Embodiment 30. The method of any one of embodiments 22 to 29, wherein the biological sample comprises less than 0.1% weight by volume (w/v) EVT.

Embodiment 31. The method of any one of embodiments 22 to 30, wherein the EVT comprise EVT residing in or passing through the cervix.

Embodiment 32. The method of any one of embodiments 22 to 31, further comprising washing the biological sample to achieve a single-cell solution.

Embodiment 33. The method of any one of embodiments 22 to 32, further comprising filtering the biological sample to achieve a single-cell solution.

Embodiment 34. The method of any one of embodiments 22 to 33, wherein the at least one biomarker is derived from a placenta-specific cell, a maternal cell, or a fetal cell.

Embodiment 35. The method of embodiment 34, wherein the placenta-specific cell is an EVT, villous trophoblast or syncytiotrophoblast cell.

Embodiment 36. The method of any one of embodiments 22 to 35, wherein the at least one biomarker comprises an EVT biomarker.

Embodiment 37. The method of embodiment 36, wherein the EVT biomarker comprises integrin subunit alpha 1 (ITGA1), cadherin 5 (CDH5), platelet and endothelial cell adhesion (PECAM1), matrix metallopeptidase 9 (MMP9), HLA-G, integrin alpha 6 (ITGA6), chorionic gonadotropin (hCG), and pregnancy-specific beta-1-glycoprotein 1 (PSG1), or a combination thereof.

Embodiment 38. The method of any one of embodiments 22 to 37, wherein the at least one biomarker comprises a placental protein.

Embodiment 39. The method of embodiment 38, where the placental protein comprises hCG, progesterone, human placental lactogen, vascular endothelial growth factor (VEGF), VEGF-receptor (FLT1), PGF, insulin-like growth factor binding protein 1 (IGFBP1 or PP12), galectin 13 (LGALS13), galectin 14 (LGALS14), vasculotropin, PAPPA, endoglin (ENG), vascular endothelial cell proliferation factor, or a combination thereof.

Embodiment 40. The method of any one of embodiments 22 to 39, further comprising identifying a cervical condition.

Embodiment 41. The method of embodiment 40, wherein identifying a cervical condition comprises determining an elevated level or a decreased level of at least one cervical health biomarker relative to a standard control.

Embodiment 42. The method of embodiment 41, wherein the at least one cervical health biomarker comprises carcinoembryonic antigen (CEA), squamous cell carcinoma antigen (SCC Ag) carbohydrate antigen 19-9 (CA19-9), KRT7, hPL, CDH5, PECAM1, ITGA1, MMP9, TGFB2, HLAG, PSG1, ITGA6, CDH1, LGALS13, LGALS14, PAPPA, PGF, AFP, FLT, ENG, Spint1, ADAM12, MPO, CD68, BMK13, CD45, or a combination thereof.

Embodiment 43. The method of any one of embodiments 40 to 42, wherein the cervical condition is cervical infection, bleeding, inflammation, or cervical cancer.

Embodiment 44. The method of any one of embodiments 22 to 43, wherein the one or more pregnancy-associated risks or conditions comprises placental dysfunction or insufficiency, pregnancy-induced hypertension, placental abruption, pregnancy loss, miscarriage, preeclampsia, eclampsia, Hemolysis Elevated Liver enzymes and Low Platelet (HELLP) syndrome, fetal growth restriction, intrauterine growth restriction, preterm birth, low birthweight, placenta percreta, placenta increta, placenta previa, gestational hypertension, gestational thrombosis, stillbirth, placental infarction, or a combination thereof.

Embodiment 45. The method of any one of embodiments 22 to 44, wherein the one or more pregnancy-associated risks or conditions comprises an early gestational complication.

Embodiment 46. The method of embodiment 45, wherein the early gestational complication is placental dysfunction or insufficiency, risk of early pregnancy loss, risk of preeclampsia, or risk of preterm birth.

Embodiment 47. The method of any one of embodiments 22 to 46, wherein the subject is a pregnant subject.

Embodiment 48. The method of any one of embodiments 22 to 47, wherein the subject is at least four weeks pregnant.

Embodiment 49. A method of identifying a pregnancy-associated risk or condition in a subject, the method comprising: a) obtaining a biological sample from the cervix of the subject, wherein the biological sample comprises extravillous trophoblast (EVT) cells and biological material derived from the cervix of the subject, wherein the biological material comprises at least 90% weight by volume (w/v) of the biological sample; b) performing single-cell time-of-flight mass spectrometry (CyTOF-MS) on the biological sample to generate an output; and c) determining an elevated level or a decreased level of at least one biomarker in the biological sample relative to a standard control based on the output, thereby identifying the one or more pregnancy-associated risks or conditions.

Embodiment 50. The method of embodiment 49, wherein the method does not include isolating the EVT cells.

Embodiment 51. The method of embodiment 49 or 50, further comprising performing flow cytometry analysis on the biological sample.

Embodiment 52. The method of any one of embodiments 49 to 51, wherein the method is completed in less than 24 hours.

Embodiment 53. The method of any one of embodiments 49 to 52, wherein the biological material comprises mucous, a maternal cell, biological fluid, or a combination thereof.

Embodiment 54. The method of any one of embodiments 49 to 53, wherein the biological sample comprises at least about 25 cells per 2 milliliters.

Embodiment 55. The method of any one of embodiments 49 to 54, wherein the biological sample comprises less than 0.1% weight by volume (w/v) EVT.

Embodiment 56. The method of any one of embodiments 49 to 55, wherein the EVT comprise EVT residing in or passing through the cervix.

Embodiment 57. The method of any one of embodiments 49 to 56, further comprising washing the biological sample to achieve a single-cell solution.

Embodiment 58. The method of any one of embodiments 49 to 57, further comprising filtering the biological sample to achieve a single-cell solution.

Embodiment 59. The method of any one of embodiments 49 to 58, wherein the at least one biomarker is derived from a placenta-specific cell, a maternal cell, or a fetal cell.

Embodiment 60. The method of embodiment 59, wherein the placenta-specific cell is an EVT, villous trophoblast or syncytiotrophoblast cell.

Embodiment 61. The method of any one of embodiments 49 to 60, wherein the at least one biomarker comprises placental growth factor (PGF), pregnancy-associated plasma protein-A (PAPP-A), galectin 13 (LGALS13), galectin 14 (LGALS14), alpha fetoprotein (AFP), endoglin (ENG), fms-like tyrosine kinase 1 (FLT), CGB, ADAM12, ADAM17, BDNF, CCL5, CRP, CXCL8, EGF, sEGFR, EPO, HBEGF, IFNG, IGF, IGFBP1, IL1B, IL6, INHA, MMP2, MMP7, MMP9, MMP12, NGF, TGA1, TGFA, TGFB2, TIMP3, TNFA, TSH, VEGF, or a combination thereof.

Embodiment 62. The method of any one of embodiments 49 to 61, wherein the at least one biomarker comprises an EVT biomarker.

Embodiment 63. The method of embodiment 36, where the EVT biomarker comprises integrin subunit alpha 1 (ITGA1), cadherin 5 (CDH5), platelet and endothelial cell adhesion (PECAM1), matrix metallopeptidase 9 (MMP9), HLA-G, integrin alpha 6 (ITGA6), chorionic gonadotropin (hCG), pregnancy-specific beta-1-glycoprotein 1 (PSG1), or a combination thereof.

Embodiment 64. The method of any one of embodiments 49 to 63, wherein the at least one biomarker comprises a placental protein.

Embodiment 65. The method of embodiment 38, where the placental protein comprises hCG, progesterone, human placental lactogen, vascular endothelial growth factor (VEGF), VEGF-receptor (FLT1), PGF, insulin-like growth factor binding protein 1 (IGFBP1 or PP12), galectin 13 (LGALS13), galectin 14 (LGALS14), vasculotropin, PAPPA, endoglin (ENG), vascular endothelial cell proliferation factor, or a combination thereof.

Embodiment 66. The method of any one of embodiments 49 to 65, further comprising identifying a cervical condition.

Embodiment 67. The method of any one of embodiments 49 to 65, wherein identifying a cervical condition comprises determining an elevated level or a decreased level of at least one cervical health biomarker relative to a standard control.

Embodiment 68. The method of embodiment 67, wherein the at least one cervical health biomarker comprises carcinoembryonic antigen (CEA), squamous cell carcinoma antigen (SCC Ag) carbohydrate antigen 19-9 (CA19-9), KRT7, hPL, CDH5, PECAM1, ITGA1, MMP9, TGFB2, HLAG, PSG1, ITGA6, CDH1, LGALS13, LGALS14, PAPPA, PGF, AFP, FLT, ENG, Spint1, ADAM12, MPO, CD68, BMK13, CD45, or a combination thereof.

Embodiment 69. The method of any one of embodiments 66 to 68, wherein the cervical condition is cervical infection, bleeding, inflammation, or cervical cancer.

Embodiment 70. In embodiments, the cervical infection is human papilloma virus infection.

Embodiment 71. The method of any one of embodiments 49 to 69, wherein the one or more pregnancy-associated risks or conditions comprises placental dysfunction or insufficiency, pregnancy-induced hypertension, placental abruption, pregnancy loss, miscarriage, preeclampsia, eclampsia, Hemolysis Elevated Liver enzymes and Low Platelet (HELLP) syndrome, fetal growth restriction, intrauterine growth restriction, preterm birth, low birthweight, placenta percreta, placenta increta, placenta previa, gestational hypertension, gestational thrombosis, stillbirth, placental infarction, or a combination thereof.

Embodiment 72. The method of any one of embodiments 49 to 71, wherein the one or more pregnancy-associated risks or conditions comprise an early gestational complication.

Embodiment 73. The method of embodiment 72, wherein the early gestational complication comprises placental dysfunction or insufficiency, risk of early pregnancy loss, risk of preeclampsia, or risk of preterm birth.

Embodiment 74. The method of any one of embodiments 49 to 73, wherein the subject is a pregnant subject.

Embodiment 75. The method of any one of embodiments 49 to 74, wherein the subject is at least four weeks pregnant.

Embodiment 76. A kit for obtaining a biological sample from a subject, comprising: a) a collection device; b) a collection container comprising a stabilizing solution.

Embodiment 77. The kit of embodiment 76, wherein the collection device comprises a cyto-brush, a scraper or a combination thereof.

Embodiment 78. The kit of embodiment 76 or 77, further comprising an antibody specific for a biomarker.

Embodiment 79. The kit of embodiment 78, wherein the biomarker is an EVT biomarker, a placental protein, or a cervical health biomarker.

Embodiment 80. The kit of any one of embodiments 76 to 79, further comprising a detectable moiety.

Embodiment 81. The kit of any one of embodiments 76 to 80, further comprising a standard control.

Embodiment 82. A kit for obtaining a cervical fluid sample from a subject, comprising: a) a collection device; b) a collection container comprising a stabilizing solution; and c) a cell lysis solution and/or a cell removal device.

Embodiment 83. The kit of embodiment 82, further comprising: a protein stabilizing solution or a nucleic acid stabilizing solution.

Embodiment 84. The kit of embodiment 82 or 83, further comprising a mucous solubilizing agent, or a mucous removal device.

Embodiment 85. The kit of any one of embodiments 82 to 84, further comprising a detection assay for detecting an elevated level or a decreased level of at least one biomarker in the cervical fluid sample.

Embodiment 86. The kit of embodiment 85, wherein the detection assay comprises a lateral flow device.

Embodiment. 87. The kit of embodiment 85 or 86, wherein the at least one biomarker is a protein, nucleic acid, cell fragment, microvesicle, ectosome, microparticle, extracellular vesicle, micelle, or combination thereof.

### EXAMPLES

### Example 1: Identification of cEVT Cells in Cervical Samples

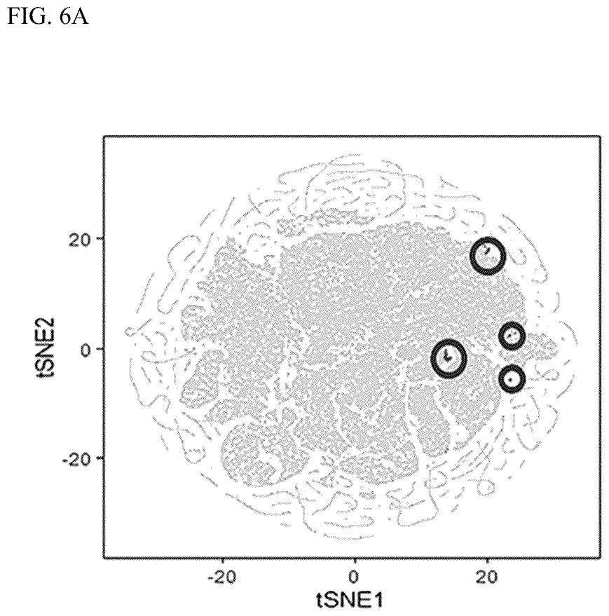

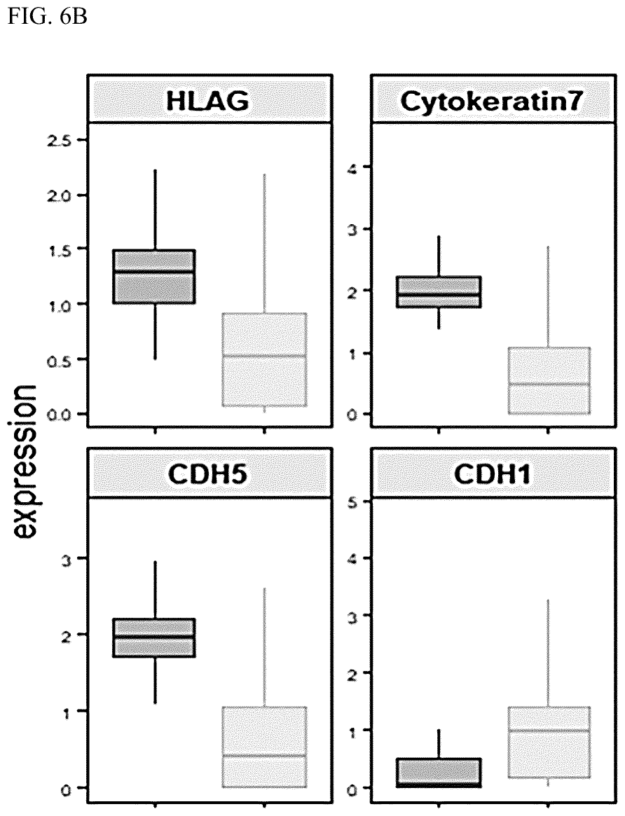

CyTOF was used to identify cEVTs in cervical samples (FIGS. 6A-6B, 7A-7B, and 8). In FIGS. 6A-6B, using three cEVT control samples, four clusters of HLA-G+ cells were identified that co-expressed the expected trophoblast lineages markers HLA-G, Cytokeratin7 and mesenchymal CDH5, but not epithelial marker CDH1. HLA-G, Cytokeratin 7, and CDH5 are shown to behave similarly, as expected, while the epithelial marker CDH1 is expressed in mostly maternal epithelial cells (bottom left, left side of FIG. 6B), and is absent in the trophoblast cells. In FIGS. 7A-7B, using one sample collected prior to a FGR (fetal growth restriction) diagnosis and one matched healthy control sample, HLA-G+cEVT cells were identified. The low frequency of cEVT cells (approximately 0.6%-0.7%) in FIG. 7B demonstrates the importance of having the ability to use low numbers of cEVT cells during analysis while retaining accuracy.

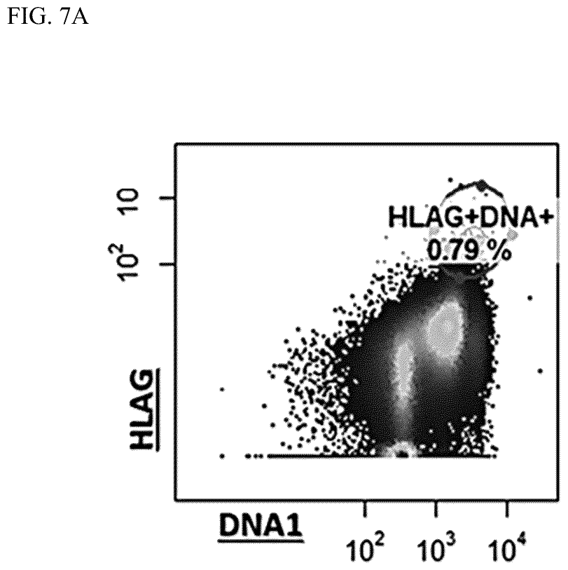

FIG. 8 demonstrates strong agreement between the cEVT protein signatures obtained by CyTOF. A comparison can be made to cEVT protein signatures obtained with IF, using the same HLA-G antibody and protein targets, as found in Bolnick, J. M. et al. Altered Biomarkers in Trophoblast Cells Obtained Noninvasively Prior to Clinical Manifestation of Perinatal Disease. Sci. Rep. 6, 32382; doi: 10.1038/srep32382 (2016), which is incorporated herein in its entirety and for all purposes. Alternative biomarkers in trophoblast cells can be obtained noninvasively prior to clinical manifestation of perinatal disease Although both CyTOF and IF can identify similar expression patterns, key benefits of the CyTOF method include expression analysis of multiple cEVT proteins occurring at the same time, less samples loss, more rapid acquisition of results, more cost-effective methods, use of far fewer cells, and lack of a need for isolation steps. Multiplexing is also enhanced when using CyTOF.

FIG. 8 shows data that clearly distinguish healthy control patients from those with abnormal outcomes by measuring cEVT proteins. cEVTs have unique protein expression signatures including lower placenta function markers (e.g., LGALS13, LGALS14, PAPPA, PGF, and AFP) and higher anti-angiogenic factors (e.g., FLT-1 or ENG). Further, results from CyTOF-based methods show similar results compared to the median expression profiles of HLA-G isolated cEVT cells from healthy and adverse pregnancies determined by IF staining, thereby confirming the validity of the methods described herein.

### Example 2: Analysis of Placental Trophoblast Cells

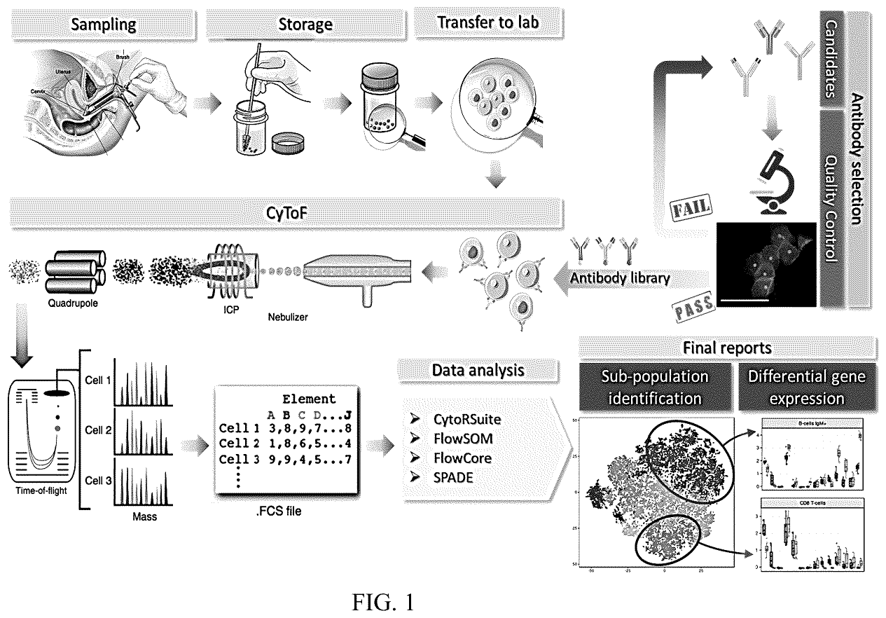

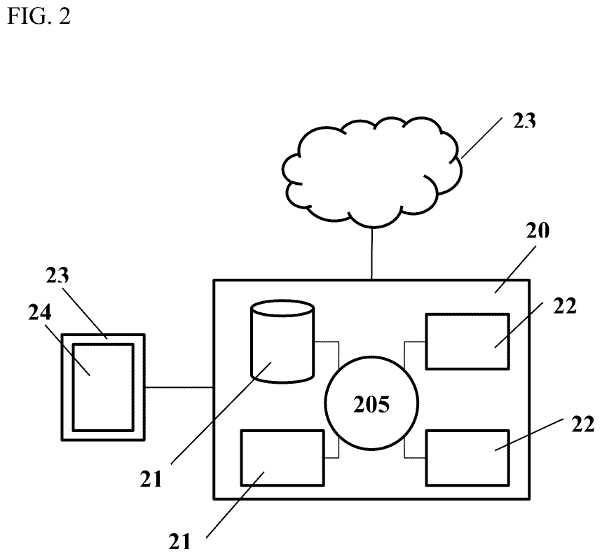

This experiment analyzed disease-specific proteins in placental trophoblast cells. Samples from pregnant patients between five and twenty weeks of gestation were obtained by safe pap smears. The samples non-invasively captured hundreds of homogeneous, HLA-G- and hCG-expressing trophoblast cells. The sample were further analyzed for indications of early gestational complications. CyTOF was used to screen clinically high-risk women in the first trimester for common perinatal complications (e.g., sPE, IUGR) caused by abnormal placental development (FIG. 1).

In this experiment, up to 100 samples of trophoblast cells in the first trimester were collected, along with de-identified medical records to determine pregnancy outcomes. Cervical cells were sampled between five and twenty weeks of pregnancy with a cytobrush during a routine speculum exam, as described in Imudia et al. Retrieval of trophoblast cells from the cervical canal for prediction of abnormal pregnancy: a pilot study. Hum Reprod. 2009 September; 24 (9): 2086-2092; doi: 10.1093/humrep/dep206 (2009), which is incorporated herein in its entirety and for all purposes. The ten-minute procedure was conducted with the patient lying on an examining table in the lithotomy position. A speculum was placed in the vagina; approximately 2 cm of the cytological brush was introduced into the external cervical os, while rotating the brush as it was removed. Contact with the cervical wall was minimized to avoid bleeding. Samples were then rinsed from the cytobrush into a solution, which stabilized the samples until transport (e.g., CytoLyt®, Hologic). Samples were stored, and quality assessed. The cells were labeled with 25 metal-conjugated antibodies that bound cell lineage- and disease-specific proteins. A comparison of proteins expressed in various trophoblast lineages shows that the cervical HLA-G+ cells are of extra-villous placental origin, as seen in Bolnick, J. M., et al. Trophoblast retrieval and isolation from the cervix (TRIC) for noninvasive prenatal screening at 5 to 20 weeks of gestation. Fertil Steril. 2014 July; 102 (1): 135-142.e6. doi: 10.1016/j.fertnstert.2014.04.008. Epub 2014 May 10. PMID: 24825422; PMCID: PMC10411519, which is incorporated herein in its entirety and for all purposes.

CyTOF mass spectrometry was used to nebulize each cervical cell in a plasma beam and count ‘events/metals’ per cell. Briefly, cells in a cervical sample were washed with Maxpar Cell Staining Buffer (MCSB, Fluidigm) and resuspended in 50 μl of MCSB. After staining, cells were washed twice, resuspended, and fixed in PFA. Fixed pellets were stored (−80° C.) for sample batching for up to 2 months.

Levels of alpha fetoprotein (AFP) and placental growth factor (PGF), along with other cell-type specific proteins such as HCGB, KRT7, hPL, CDH5, PECAM1, ITGA1, MMP9, TGFB2, HLA-G, PSG1, ITGA6, CDH1, LGALS13, LGALS14, PAPPA, PGF, AFP, FLT1, ENG, SPINT1, ADAM12, MPO, CD68, BMK13, and CD45 were measured using placental cells in cervical specimens in the first trimester, comparing cohorts of subjects with normal term pregnancies to those with adverse outcomes associated with low birth weight for gestational age (GA) at birth. Single-cell mass spectrometry assays (MSAs) were used to quantify protein levels in trophoblast cells and optimize the assay with clinical specimens.

The remaining patient cervical samples were assayed and protein levels and compared to patient outcomes, particularly reduced birth weight for gestational age.

Selected putative biomarkers in cervical EVT cells obtained in the between 5-20 weeks GA were quantified, and cohorts of subjects with normal term pregnancies were compared with cohorts of subjects with adverse outcomes associated with low birth fetal weight for GA at birth. The EVT cell specific HLAG protein marker was used for cell identification, and a non-affected control protein (KRT7) was used for normalization.

### Example 3: Identification of Pregnancies with Abnormal Placentation

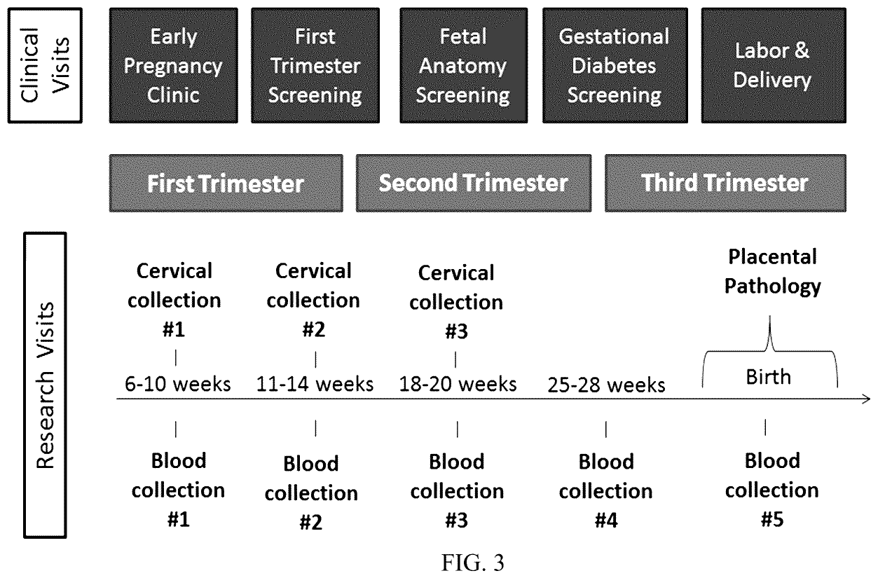

This experiment demonstrates the identification of pregnancies with abnormal placentation. A diagram of the study's sample collection schedule, which enrolls 500 patients, is found in FIG. 3.

Patients are selected from an appointments list. High-risk patients are defined based on reproductive and medical history as women with a previous pregnancy/placental complication, previous sPE, infertility treatment, advanced maternal age (>35 yrs old), BMI>35 kg/m², and/or a maternal-related complication. cEVT and blood sampling occurs during clinic appointments, minimizing the burden of research study visits and maximizing subject retention. Exclusion criteria include subjects less than 18 years of age or subjects carrying multiple fetuses. Among the pregnant women, half (˜250 subjects) have male fetuses and half (˜250 subjects) have female fetuses. Differences based on sex of the fetus are controlled for in all analyses.

Patient risks (both low risks and high risks) of abnormal placentation are assessed. At 6-10 weeks gestational age (GA), viable singleton pregnancies with normal fetal development and normal nuchal translucency/nasal bone/early anatomy are selected for further study. Cervical swabs and blood collection are performed as outlined in FIG. 1. To determine the risk of placental abnormalities or an abnormal outcome (e.g., sPE), the following characteristics are measured/assessed: 1) crown rump length assessment to determine GA for evaluation of fetal growth; 2) clinical biomarkers are measured in the maternal blood; 3) mode of conception (e.g., natural vs. fertility treatment) is recorded; 4) presence of vaginal bleeding greater than three days (e.g., red, less, none) is recorded; 5) a sub-chorionic hematoma evident on ultrasound is recorded; and 6) Doppler results of the uterine arteries are evaluated. At 18-20 weeks GA, during standard fetal anatomic screening, data is collected on uterine artery Doppler and placental size following ultrasound. Patients are followed throughout pregnancy and all relevant pregnancy data is recorded in a secure REDCap database. Additionally, standardized pathological reports on the placenta in the study population are recorded at birth, and clinical data is used for longitudinal assessment and correlation purposes with the CyTOF and biomarker data.

### Example 4: CEVT Protein Expression Using CyTOF

This experiment demonstrates the ability to define cell phenotypes, transcriptional profiles, and functional protein regulation in a cervical sample looking at 40+ biomarkers in less than 24 hours.

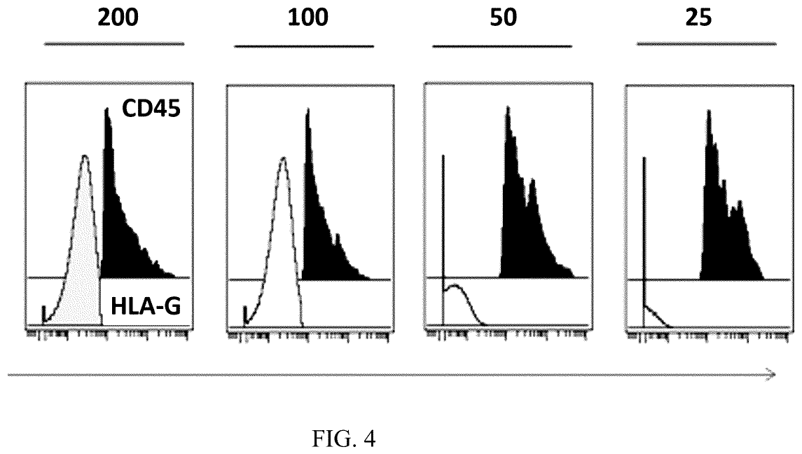

Cervical samples are prepared from recruited patients as described above. Individual samples are stained with the cEVT Profiling Assay antibody cocktail using a standard staining protocol. For multiplexing, samples are barcoded. Each experiment contains spike-in controls to maintain reproducibility. Also for reproducibility, automated tuning is used (calibration of the CyTOF instrument) for consistency, and normalization beads are spiked into samples to monitor and correct for variations during acquisition. Unbound antibodies are removed using filters before injecting a sample into the CyTOF machine. Fixed cells can be stored (−80° C.) for sample batching in FBS with 10% DMSO for up to 2 months. Cell acquisition is optimized to 300-600 events per second to maximize cell recovery and signal acquisition for rare cell populations. This CyTOF method reliably identifies as few as 25 cells in a specimen (FIG. 4). On average there are ˜750 cEVTs/cervical sample.

Required antibodies are prelabeled with metal isotopes by Fluidigm. All other antibodies are labeled with a specific metal isotope using Fluidigm's MaxPar™ antibody labeling kit. Protein markers with low expression to high signal intensity metal isotopes are conjugated for improved resolution. Isotope purity is monitored to ensure data quality. Protein markers are chosen exclusively for known cell populations (e.g., CDH1 vs. CDH5) to further minimize signal interference.

MaxPar™ antibody labeling adds a metal polymer tag by disulfide reduction (typically in the antibody Fc region) via a maleimide linker. This labeling approach is efficient: ˜ 100 metal ions/antibody in about four hours. Unbound metal ions can produce significant background and so are removed using a 50 kDA filter. Labeling success and background are tested using polystyrene beads that can bind a limited number of antibodies and be run on CyTOF to evaluate performance.

Antibody validation and titration is standard for flow cytometry and CyTOF. In brief, antibodies are obtained from commercial sources that provide QC and the antibodies are titered to maximize signal-to-noise ratio and confirm CyTOF specificity using trophoblast like cell lines (HTR-8/SVneo, Jeg3, HEC-1A from ATCC).

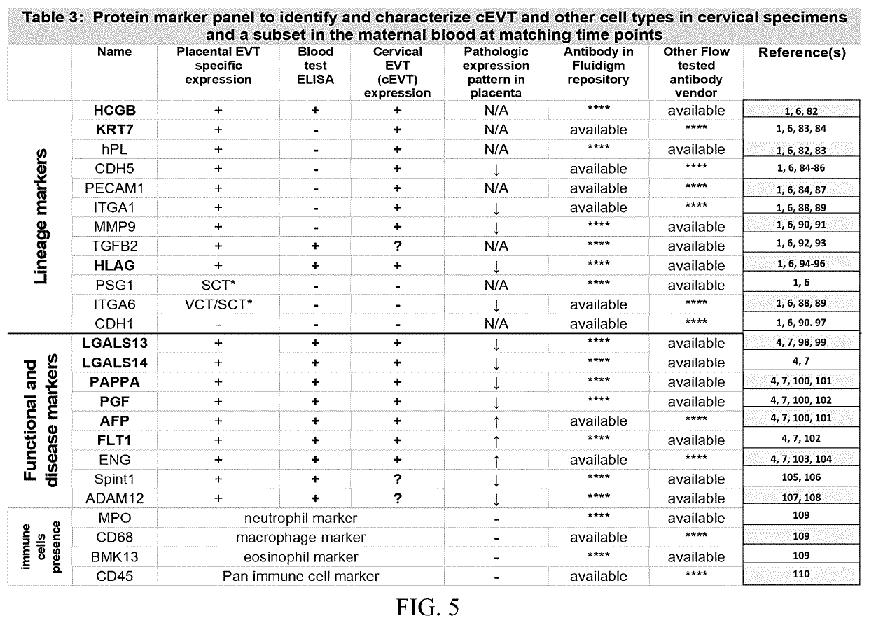

Once all antibodies are labeled and titrated, the panel (FIG. 5) is assembled to create the cEVT Profiling Assay consisting of 25 antibodies against the lineage proteins, functional proteins from our published studies, and immune cell markers. The last step is to ensure that cross-reactivity is limited, and specificity maintained. Fluidigm's standard protocol is used to ensure reliable CyTOF data (FLDM-400247 Rev 01 WHITE PAPER). To improve data resolution, antibodies are tested as single reactions and as minus-one controls (omitting only one antibody in the panel). Metal tags used to bind antibodies are not 100% pure, so this approach improves data quality.

Precision and accuracy of the cEVT Profiling Assay is tested in: 1) cell lines (HTR-8/SVneo, Jeg3, HEC-1A); 2) synthetic samples; 3) clinical sample pools; and 4) single samples. Intra-assay reproducibility, intermediate precision, and accuracy are all assessed. Mean, standard deviation, coefficient of variation, and confidence interval of the mean for the quantified populations are also calculated.

To identify differential abundance, the proportions of cell types (maternal, placental, immune) are compared across experimental groups (affected vs. healthy pregnancies) to highlight populations present at different ratios. To calculate and compare proportions, cell counts by cluster and sample are transformed and used in a generalized linear mixed model, with the assumption that for a given cell population cell counts follow a binomial distribution. A threshold filter is applied to remove samples of <5 cells to avoid errors during model fitting. Differential analysis of protein marker expression is conducted within each cluster by calculating the median expression of all markers in each cell population and sample. This data is used as the response variable in a linear model, with the assumption that the median marker expression follows a Gaussian distribution. A possible drawback from using a summary statistic is the level of uncertainty, which increases as the number of cells used to calculate the summary statistic decreases. This is overcome by assigning observation weights (cell number) to each cluster and sample in the statistical model. Finally, using subpopulation abundance and protein expression profiles, SPADE is used to visualize similarity among clusters as a minimum-spanning tree.

### Example 5: Detection of Pregnancy-Associated Plasma Protein A in Cervical Fluid Samples

Applicant has developed an approach to detecting PAPP-A (Pregnancy-Associated Plasma Protein A) in substantially cell-free cervical specimens during the first trimester of pregnancy, a novel method not traditionally employed in current practices which typically utilize blood samples at later stages. PAPP-A, essential for placental development and fetal growth, is a key indicator in prenatal screenings for chromosomal abnormalities such as Down syndrome (Trisomy 21) and Edwards syndrome (Trisomy 18).

This methods allows for early detection of PAPP-A levels from cervical specimens, as early as the first trimester, offering a new window into assessing placental health and potential pregnancy complications. Low levels of PAPP-A are associated with increased risks of adverse outcomes, including intrauterine growth restriction (IUGR), pre-eclampsia, and preterm birth, due to its critical role in placental function.

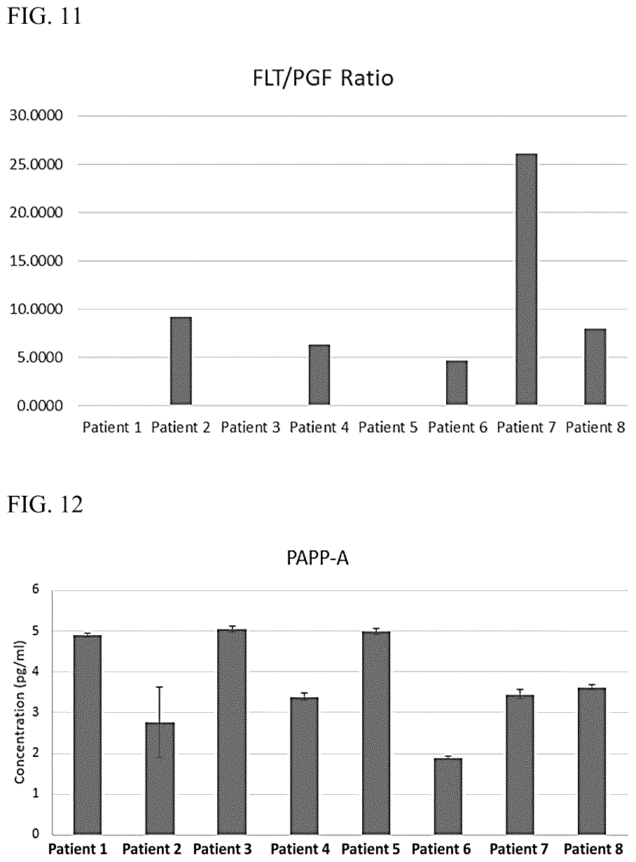

In this instance, Patient 6's data, obtained from cervical samples in the first trimester, showed a marked decrease in PAPP-A levels compared to healthy controls, indicating possible placental complications (FIG. 12). This early detection method could significantly enhance prenatal care by providing earlier insights into placental health and fetal development.

### Example 6: Detection of Alpha-Fetoprotein in Cervical Fluid Samples

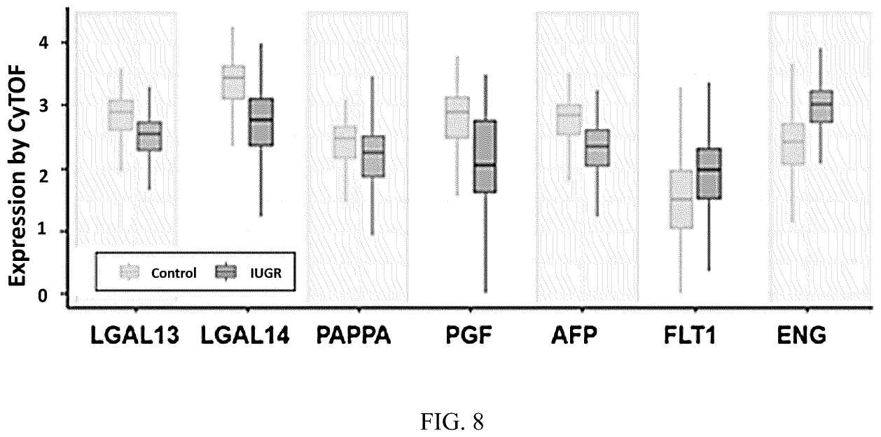

Applicant developed a prenatal diagnostic for detection of Alpha-fetoprotein (AFP) in cervical fluid samples from eight pregnant patients during the first trimester (FIG. 10). Traditionally, AFP testing is conducted via maternal blood samples in the second trimester (around 15-20 weeks of gestation) for various screening purposes. The early detection of AFP in cervical fluid samples as demonstrated here could parallel the indications used in blood tests, with potential applications in:

1) Screening for Neural Tube Defects (NTDs): Elevated AFP levels in maternal serum are linked to NTDs in the fetus, such as spina bifida and anencephaly. This early detection in cervical specimens could offer a new approach to screening for these conditions well before the conventional second-trimester timeline. 2) Screening for Chromosomal Abnormalities: AFP is a critical component of the “triple” or “quadruple” screen, assessing the risk of chromosomal abnormalities like Down syndrome and Edwards syndrome. The ability to detect AFP levels in the first trimester from cervical specimens may provide earlier insights into these risks. 3) Assessment of Other Pregnancy Complications: Abnormal AFP levels can indicate issues such as placental problems or fetal growth restrictions. Early detection from cervical fluid samples could facilitate prompt intervention and management of these complications.

This method of detecting AFP in cervical fluid samples during the first trimester enables earlier and more comprehensive screening in pregnancy and allows diagnosis of fetal conditions and pregnancy complications early in pregnancy.

### Example 7: Detection of Placental Growth Factor in Cervical Fluid Samples

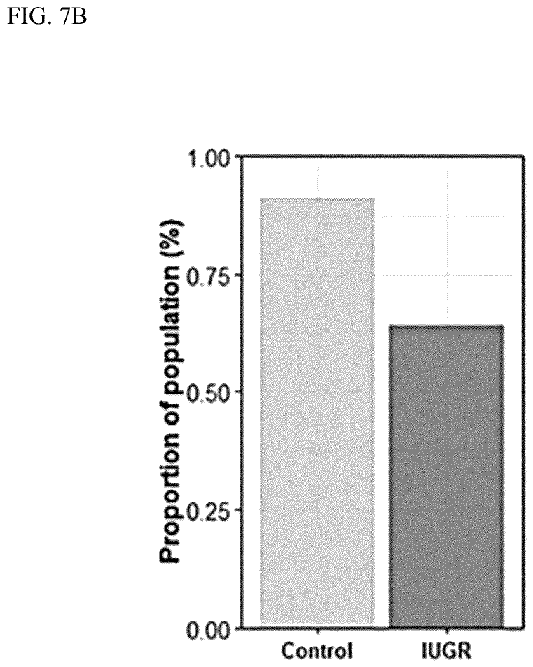

Applicant developed a cell-free detection method for identifying Placental Growth Factor (e.g. PGF, PLGF) in first-trimester cervical fluid samples from eight pregnant patients, using ELISA (Enzyme-Linked Immunosorbent Assay). As illustrated in FIG. 9, PLGF was detected in all eight patients tested. PLGF is essential in assessing preeclampsia risk, and is typically monitored in maternal blood. The 100% detection rate in early-stage cervical fluid samples marks a significant advancement, offering earlier insights into preeclampsia risk. This method enables less invasive, early detection of key placental biomarkers.

### Example 8: Detection of Soluble FMS-Like Tyrosine Kinase-1 (sFlt-1) in Cervical Fluid Samples

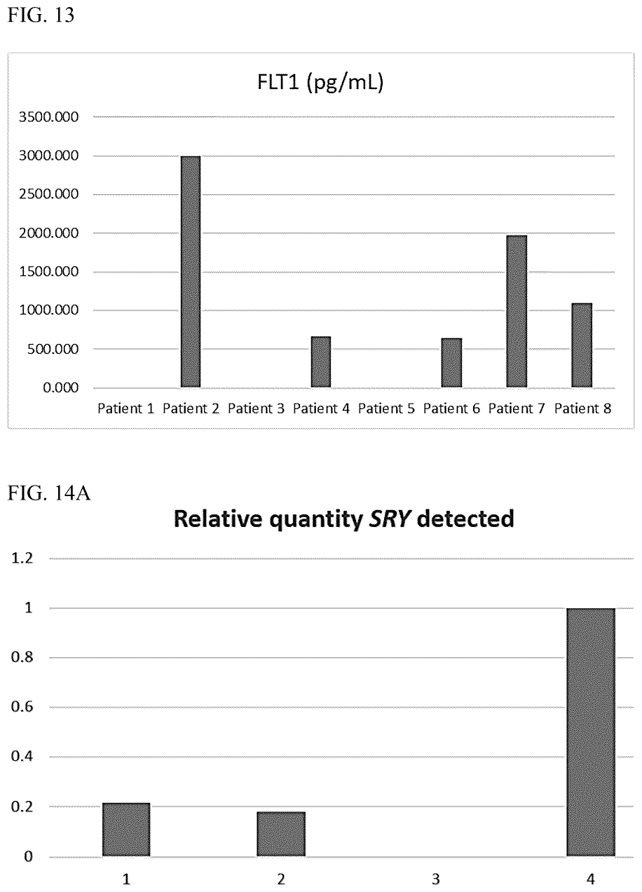

Applicant detected soluble FMS-like tyrosine kinase-1 (sFlt-1) in cervical fluid samples from 5 out of 8 pregnant patients during the first trimester of pregnancy (FIG. 13). sFlt-1, a crucial biomarker for preeclampsia, is an anti-angiogenic protein produced by the placenta, influencing blood vessel formation. The detection of sFlt-1 in these early-stage cervical fluid samples represents a significant development in prenatal screening, particularly for the early identification of preeclampsia risk. This method's ability to detect sFlt-1 in a substantial proportion of samples (5 out of 8) allows for a less invasive, early screening tool for preeclampsia, thereby enhancing prenatal care and risk assessment.

### Example 9: Identification of High-Risk Pregnancy by Measuring sFlt-1/PLGF Ratio in Cervical Fluid Samples

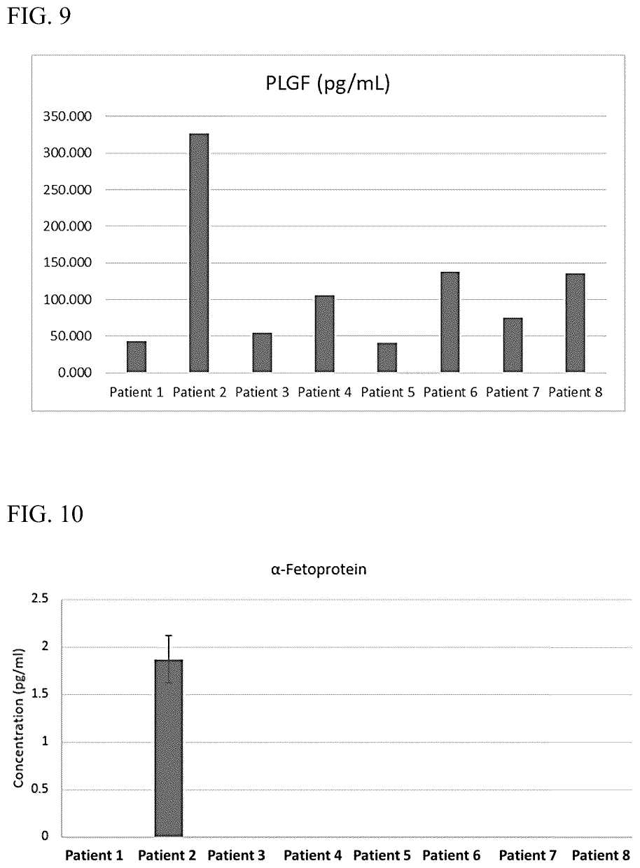

The sFlt-1/PlGF (PGF) ratio which compares the levels of anti-angiogenic sFlt-1 to pro-angiogenic PlGF, is instrumental in predicting the risk or presence of preeclampsia. A high sFlt-1/PlGF ratio, indicating increased anti-angiogenic activity and reduced pro-angiogenic factors, is a strong marker for precclampsia. Applicant developed a diagnostic tool for preeclampsia by measuring the sFlt-1/PlGF ratio using cervical fluid sample collected from pregnant patients in the first trimester. This method was applied to cervical fluid samples from seven healthy pregnancies and one abnormal case (Patient 7) (FIG. 11). Notably, Patient 7 exhibited the highest sFlt-1/PlGF ratio, successfully identifying the at-risk pregnancy weeks before the onset of clinical symptoms and earlier than traditional blood tests. This early detection capability highlights the potential of cervical samples in prenatal screening, and allows a significant advantage in early intervention and management of preeclampsia.

### Example 10: Identification of Fetal Sex by Cell-Free Methods

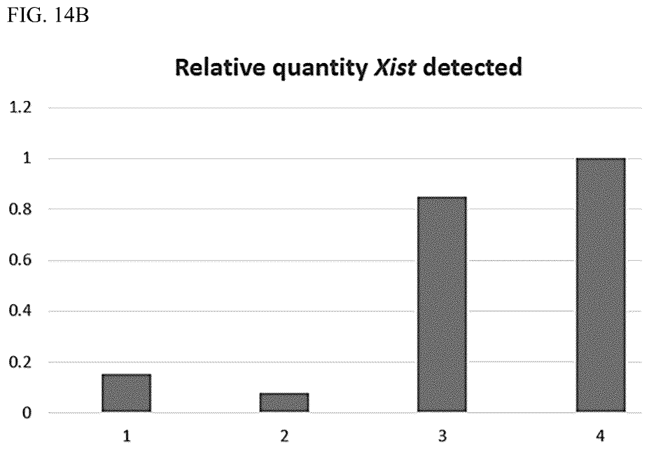

A method for non-invasive prenatal determination of fetal sex using cell-free supernatants from cervical fluid samples collected in the first trimester was developed by Applicant. The method employs PCR with TaqMan probes, targeting the SRY gene, a male-specific sequence. Detection of the XIST gene by PCR serves as a positive control. The three male fetuses and one female fetus were correctly genotyped and validated by medical records (FIGS. 14A-14B).

1. A method of identifying a pregnancy-associated risk or condition in a subject, the method comprising:
obtaining a cervical fluid sample from a subject, the cervical fluid sample comprising no greater than 1 cell per 1 milliliter (mL) volume; and
detecting an elevated level or a decreased level of at least one biomarker in the cervical fluid sample relative to a standard control, thereby identifying the pregnancy associated risk or condition.

2. (canceled)

3. The method of claim 1, wherein the cervical fluid sample is cell free or substantially cell free.

4. The method of claim 1, wherein the at least one biomarker is derived from a placenta-specific cell, a maternal cell, or a fetal cell.

5. The method of claim 4, wherein the placenta-specific cell is an extravillous trophoblast (EVT), villous trophoblast or syncytiotrophoblast cell.

6. The method of claim 1, wherein the at least one biomarker is a protein, nucleic acid, cell fragment, microvesicle, ectosome, microparticle, extracellular vesicle, micelle, or combination thereof.

7.-8. (canceled)

9. The method of claim 1, wherein the at least one biomarker comprises an EVT biomarker.

10. (canceled)

11. The method of claim 1, wherein the at least one biomarker comprises a placental protein.

12.-13. (canceled)

14. The method of claim 1, further comprising identifying a cervical condition, wherein identifying a cervical condition comprises determining an elevated level or a decreased level of at least one cervical health biomarker relative to a standard control.

15.-16. (canceled)

17. The method of claim 14, wherein the cervical condition is cervical infection, bleeding, inflammation, or cervical cancer.

18. (canceled)

19. The method of claim 1, wherein the pregnancy-associated risk or condition comprises placental insufficiency, pregnancy-induced hypertension, placental abruption, pregnancy loss, miscarriage, preeclampsia, eclampsia, Hemolysis Elevated Liver enzymes and Low Platelet (HELLP) syndrome, fetal growth restriction, intrauterine growth restriction, preterm birth, low birthweight, placenta percreta, placenta increta, placenta previa, gestational hypertension, gestational thrombosis, stillbirth, placental infarction, or a combination thereof.

20.-21. (canceled)

22. A method of identifying one or more pregnancy-associated risks or conditions in a subject, the method comprising:
a) obtaining a biological sample from the cervix of the subject, wherein the biological sample comprises extravillous trophoblast (EVT) cells;
b) performing single-cell time-of-flight mass spectrometry (CyTOF-MS) on the biological sample to generate an output; and
c) determining an elevated level or a decreased level of at least one biomarker in the biological sample relative to a standard control based on the output.

23. The method of claim 22, wherein the method does not comprise isolating the EVT cells.

24. The method of claim 22, further comprising performing flow cytometry on the biological sample.

25. (canceled)

26. The method of claim 22, wherein the biological sample further comprises biological material derived from the cervix of the subject.

27. (canceled)

28. The method of claim 26, wherein the biological material comprises at least 90% weight by volume (w/v) of the biological sample.

29. The method of claim 22, wherein the biological sample comprises at least about 25 cells per 2 mL volume.

30. The method of claim 22, wherein the biological sample comprises less than 0.1% weight by volume (w/v) EVT.

31. (canceled)

32. The method of claim 22, further comprising washing the biological sample to achieve a single-cell solution.

33. The method of claim 22, further comprising filtering the biological sample to achieve a single-cell solution.

34. The method of claim 22, wherein the at least one biomarker is derived from a placenta-specific cell, a maternal cell, or a fetal cell.

35. The method of claim 34, wherein the placenta-specific cell is an EVT, villous trophoblast or syncytiotrophoblast cell.

36. The method of claim 22, wherein the at least one biomarker comprises an EVT biomarker.

37. (canceled)

38. The method of claim 22, wherein the at least one biomarker comprises a placental protein.

39. (canceled)

40. The method of claim 22, further comprising identifying a cervical condition, wherein identifying a cervical condition comprises determining an elevated level or a decreased level of at least one cervical health biomarker relative to a standard control.

41.-42. (canceled)

43. The method of claim 40, wherein the cervical condition is cervical infection, bleeding, inflammation, or cervical cancer.

44. The method of claim 22, wherein the one or more pregnancy-associated risks or conditions comprises placental dysfunction or insufficiency, pregnancy-induced hypertension, placental abruption, pregnancy loss, miscarriage, preeclampsia, eclampsia, Hemolysis Elevated Liver enzymes and Low Platelet (HELLP) syndrome, fetal growth restriction, intrauterine growth restriction, preterm birth, low birthweight, placenta percreta, placenta increta, placenta previa, gestational hypertension, gestational thrombosis, stillbirth, placental infarction, or a combination thereof.

45.-48. (canceled)

49. A method of identifying a pregnancy-associated risk or condition in a subject, the method comprising:
a) obtaining a biological sample from the cervix of the subject, wherein the biological sample comprises extravillous trophoblast (EVT) cells and biological material derived from the cervix of the subject, the biological material comprising at least 90% weight by volume (w/v) of the biological sample;
b) performing single-cell time-of-flight mass spectrometry (CyTOF-MS) on the biological sample to generate an output; and
c) determining an elevated level or a decreased level of at least one biomarker in the biological sample relative to a standard control based on the output, thereby identifying the one or more pregnancy-associated risks or conditions.

50. The method of claim 49, wherein the method does not include isolating the EVT cells.

51. The method of claim 49, further comprising performing flow cytometry on the biological sample.

52.-75. (canceled)

76. A kit for obtaining a biological sample from a subject, comprising:
a collection device;
a collection container comprising a stabilizing solution.

77.-87. (canceled)
